(19) United States
(12) Patent Application Publication
(10) Pub. No.: US 2010/0298270 A1
(43) Pub. Date: Nov. 25, 2010

(54) Novel Compounds and Methods of Using Them

(75) Inventors: John F.W. Keana, Eugene, OR (US); Peter Ordentlich, San Diego, CA (US); Robert Goodenow, San Clemente, CA (US)

Correspondence Address: WILSON, SONSINI, GOODRICH & ROSATI, 650 PAGE MILL ROAD, PALO ALTO, CA, 94304-1050, US

(73) Assignee: SYNDAX PHARMACEUTICALS, INC., San Diego, CA (US)

(21) Appl. No.: 12/670,385

(22) PCT Filed: Jul. 23, 2008
(86) PCT No.: PCT/US08/70884

§ 371 Date: Jul. 7, 2010

Related U.S. Application Data
(60) Provisional application No. 60/951,433, filed on Jul. 23, 2007.

(51) Int. Cl. A61K 31/675; C07D 401/12; A61K 31/444; A61P 35/00; C07D 213/89; A61K 31/4412; C07D 405/12; A61K 31/4433; C07D 309/40; A61K 31/351; C07F 9/30; A61K 31/662; C07D 413/12; A61K 31/5377

(52) U.S. Cl. 514/89; 546/261; 514/335; 546/291; 514/346; 546/282.1; 514/336; 549/418; 514/460; 546/22; 560/29; 514/119; 544/131; 514/235.5

(57) ABSTRACT

Described herein are novel HDAC modulators, formulations containing them and methods of using them. In some embodiments, the HDAC modulators possess specific stereo chemistry. In other embodiments, the compounds described herein are used in the treatment or prevention of histone deacetylase mediated disorders.

### CROSS-REFERENCE

This application claims the benefit of U.S. Provisional Application No. 60/951,433, filed Jul. 23, 2007, which is incorporated herein by reference in its entirety.

### BACKGROUND OF THE INVENTION

DNA in eukaryotic cells is tightly complexed with proteins to form chromatin. Histones are small proteins that are tightly complexed with DNA to form a nucleosome, which is further connected by linker DNA to form a solenoid. Histones extending from the nucleosomal core are enzymatically modified, affecting chromatin structure and gene expression. The study of modulators of histone deacetylases (HDACs) indicates that these enzymes play an important role in cell proliferation and differentiation. The apparent involvement of HDACs in the control of cell proliferation and differentiation suggests that aberrant HDAC activity may play a role in cancer.

Histone hyperacetylation by HDAC inhibition neutralizes the positive charge of the lysine side chain, and is associated with change of the chromatin structure and the consequential transcriptional activation of a number of genes. It is believed that one outcome of histone hyperacetylation is induction of the Cyclin-dependent kinase inhibitory protein, P21, which causes cell cycle arrest. HDAC inhibitors such as Trichostatin A (TSA) and suberoylanilide hydroxamic acid (SAHA) have been reported to inhibit cell growth, induce terminal differentiation in tumor cells and prevent the formation of tumors in mice. HDAC's have been viewed as attractive targets for anticancer drug development with their ability to block angiogenesis and cell cycling, and promote apoptosis and differentiation.

Compounds and compositions capable of inhibiting histone deacetylating enzymes and inducing differentiation are useful as therapeutic or ameliorating agents for diseases that are involved in cellular growth such as malignant tumors, autoimmune diseases, skin diseases, infections, other anti-proliferative therapies, etc. HDAC inhibitors are able to target the transcription of specific disease-causing genes as well as improve the efficacy of existing cytostatics (such as the retinoids). Due to its role in the transcriptional mechanism to affect the gene expression, HDAC inhibitors are also useful as a therapeutic or prophylactic agent for diseases caused by abnormal gene expression such as inflammatory disorders, diabetes, diabetic complications, homozygous thalassemia, fibrosis, cirrhosis, acute promyelocytic leukaemia (APL), organ transplant rejections, autoimmune diseases, protozoal infections, tumors, etc.

### SUMMARY OF THE INVENTION

The present invention relates to novel substituted aromatic compounds and their pharmaceutically acceptable salts, prodrugs, solvates, polymorphs, tautomers and isomers. The compounds described herein may be used to inhibit deacetylases. The compounds described herein may be used to inhibit histone deacetylases (HDACs). The present invention also relates to compositions comprising novel substituted aromatic compounds and their pharmaceutically acceptable salts, prodrugs, solvates, polymorphs, tautomers and isomers. The present invention also relates to methods for inhibiting deacetylases. The methods described herein may be used for inhibiting histone deacetylases (HDACs). The present invention also relates to methods useful in the treatment of diseases. The compounds and compositions described herein may be useful in the treatment of diseases. The compounds described herein may be useful in the treatment of diseases such as cancer and other hyperproliferative diseases.

Compounds of Formula I, pharmaceutically acceptable salts, pharmaceutically active metabolites, pharmaceutically acceptable prodrugs, and pharmaceutically acceptable solvates thereof, may modulate the activity of HDAC enzymes; and, as such, are useful for treating diseases or conditions in which aberrant HDAC enzyme activity contributes to the pathology and/or symptoms of a disease or condition.

Described herein are compounds of Formula I:

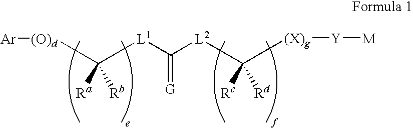

or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is:

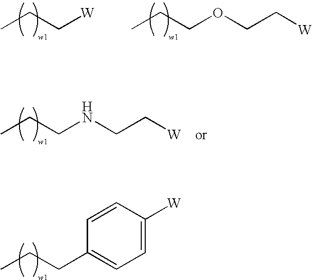

where W is selected from:

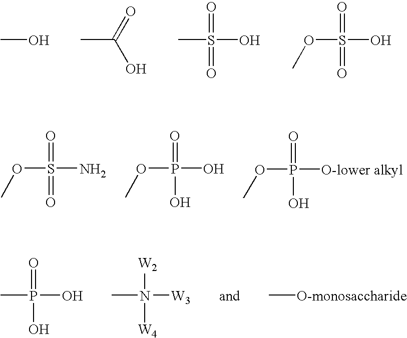

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is:

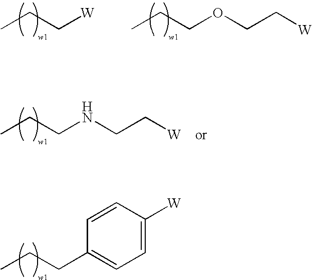

where W is selected from:

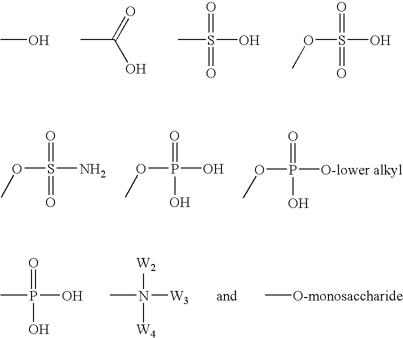

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂;
X is

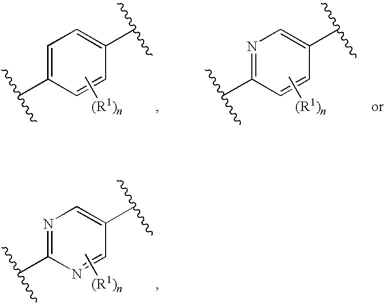

wherein each R¹ is independently halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂,
n is 0, 1, 2, 3 or 4, and the water solubilizing group is:

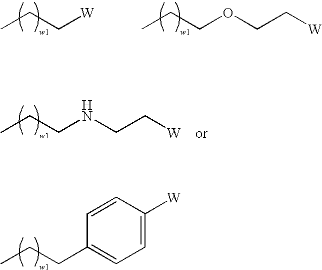

where W is selected from:

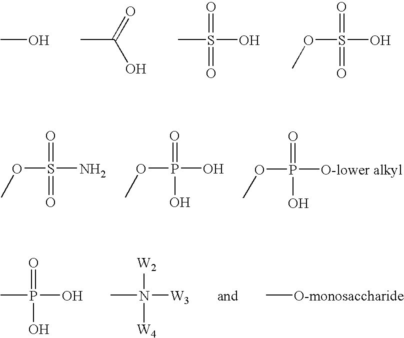

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from

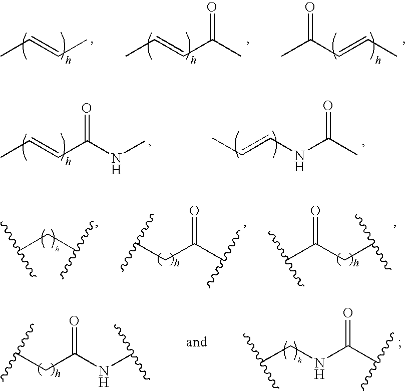

wherein h is 0, 1, 2, 3 or 4;
M is selected from

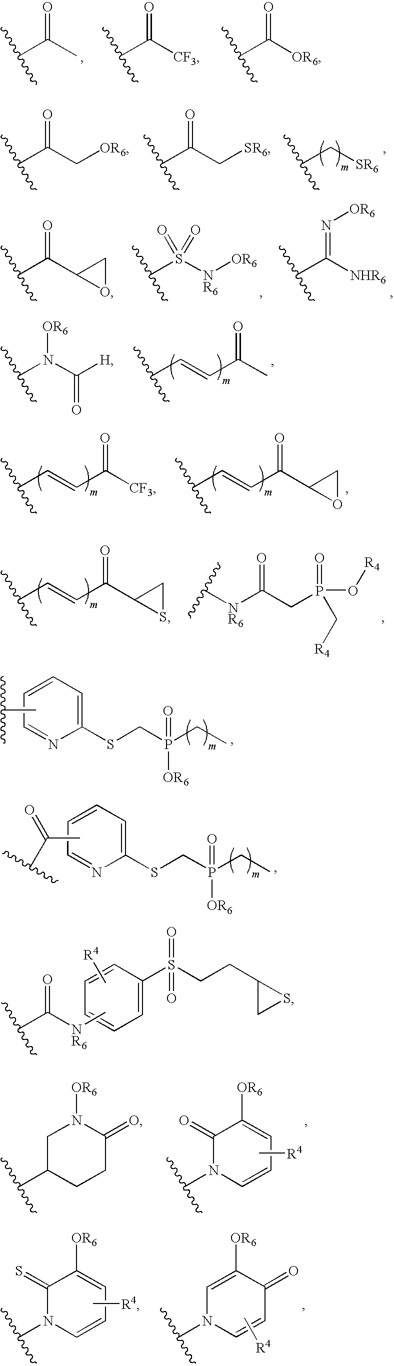

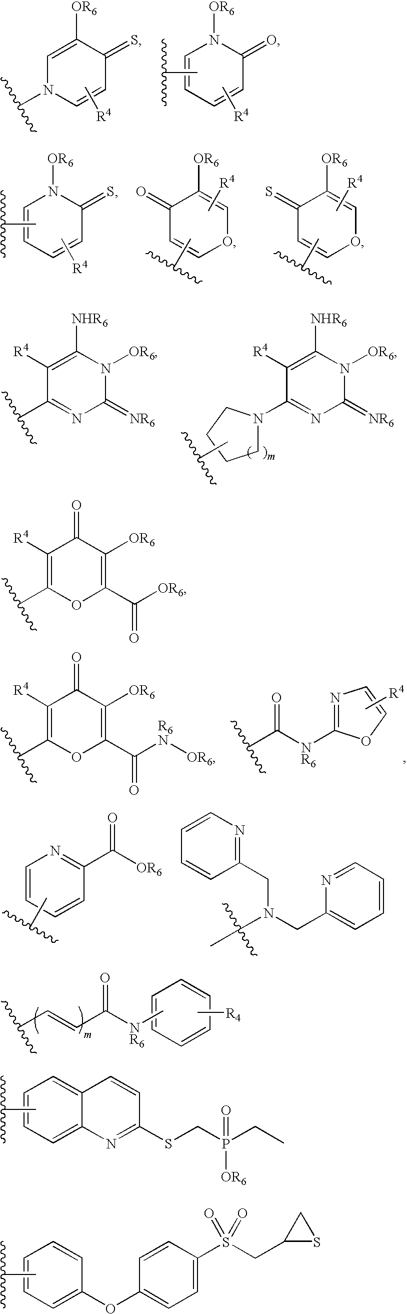

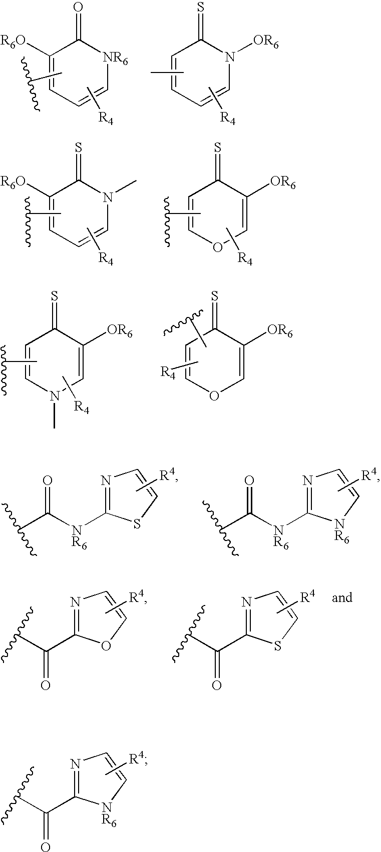

wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is:

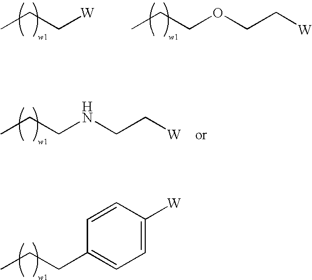

where W is selected from:

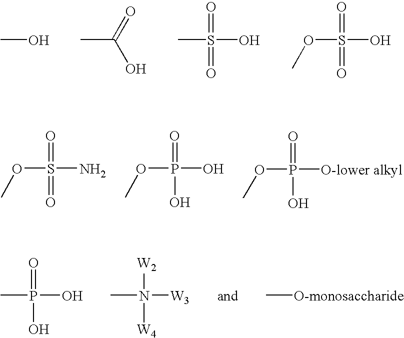

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R⁶ is hydrogen, lower alkyl, lower acyl, lower alkoxycarbonyl, carbamoyl, —PO₃H₂, and —SO₃H; and
m=0 or 1 or 2.

In a preferred embodiment, the invention provides for compounds of Formula I and their pharmaceutically acceptable salts. In further or additional embodiments, the invention provides for compounds of Formula I and their pharmaceutically acceptable solvates. In further or additional embodiments, the invention provides for compounds of Formula I and their pharmaceutically acceptable polymorphs. In further or additional embodiments, the invention provides for compounds of Formula I and their pharmaceutically acceptable esters. In further or additional embodiments, the invention provides for compounds of Formula I and their pharmaceutically acceptable tautomers. In further or additional embodiments, the invention provides for compounds of Formula I and their pharmaceutically acceptable prodrugs.

Provided herein are pharmaceutical composition comprising a compound of Formula I or a pharmaceutically acceptable salt, prodrug, solvate, polymorph, tautomer or isomer thereof. In various embodiments, the pharmaceutical composition comprises at least one pharmaceutically acceptable carrier.

Provided herein are methods for treating a patient suffering from a histone deacetylase mediated disorder, comprising administering to said individual an effective amount of a composition comprising a compound of Formula I or a pharmaceutically acceptable salt, prodrug, solvate, polymorph, tautomer or isomer thereof. In some embodiments, the compound of Formula I is administered in combination with an additional cancer therapy. In some embodiments, the additional cancer therapy is selected from surgery, radiation therapy, and administration of at least one chemotherapeutic agent. In various embodiments, the administration of the compound of Formula I occurs after surgery. In other embodiments, the administration of the compound of Formula I occurs before surgery. In some embodiments, the histone deacetylase mediated disorder is selected from the group consisting of inflammatory diseases, infections, autoimmune disorders, stroke, ischemia, cardiac disorder, neurological disorders, fibrogenetic disorders, proliferative disorders, hyperproliferative disorders, tumors, leukemias, neoplasms, cancers, carcinomas, metabolic diseases and malignant diseases. In other embodiments, the histone deacetylase mediated disorder is a hyperproliferative disease. In some embodiments, the histone deacetylase mediated disorder is cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell line cancer. In yet other embodiments, the histone deacetylase mediated disorder is a proliferative disease selected from psoriasis, restenosis, autoimmune disease, or atherosclerosis.

Provided herein are methods for degrading, inhibiting the growth of or killing cancer cells comprising contacting the cells with an amount of a composition effective to degrade, inhibit the growth of or kill cancer cells, the composition comprising a compound of Formula I or a pharmaceutically acceptable salt, prodrug, solvate, polymorph, tautomer or isomer thereof. In some embodiments, the cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell line cancer. In some embodiments, the cancer cells comprise brain, breast, lung, ovarian, pancreatic, prostate, renal, or colorectal cancer cells.

Provided herein are methods of inhibiting tumor size increase, reducing the size of a tumor, reducing tumor proliferation or preventing tumor proliferation in an individual comprising administering to said individual an effective amount of a composition to inhibit tumor size increase, reduce the size of a tumor, reduce tumor proliferation or prevent tumor proliferation, the composition comprising a compound of Formula I or a pharmaceutically acceptable salt, prodrug, solvate, polymorph, tautomer or isomer thereof. In some embodiments, the tumor occurs in the brain, breast, lung, ovaries, pancreas, prostate, kidney, colon or rectum. In some embodiments the compound of Formula I is administered in combination with an additional cancer therapy including, but not limited to surgery, radiation therapy, and administration of at least one chemotherapeutic agent. In some embodiments, the composition is administered before surgery. In other embodiments, the composition is administered after surgery.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Certain Chemical Terminology

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the Internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, —CH₂O— is equivalent to —OCH₂—.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (tram) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. The compounds presented herein include racemic mixtures, in all ratios, of stereoisomeric, regioisomeric, diastereomeric, enantiomeric, and epimeric forms. Techniques for inverting or leaving unchanged a particular stereocenter, and those for resolving mixtures of stereoisomers, or racemic mixtures, are well known in the art and it is well within the ability of one of skill in the art to choose an appropriate method for a particular situation. See, for example, Furniss et al. (eds.), VOGEL'S ENCYCLOPEDIA OF PRACTICAL ORGANIC CHEMISTRY 5.sup.TH ED., Longman Scientific and Technical Ltd., Essex, 1991, 809-816; and Heller, Acc. Chem. Res. 1990, 23, 128.

The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

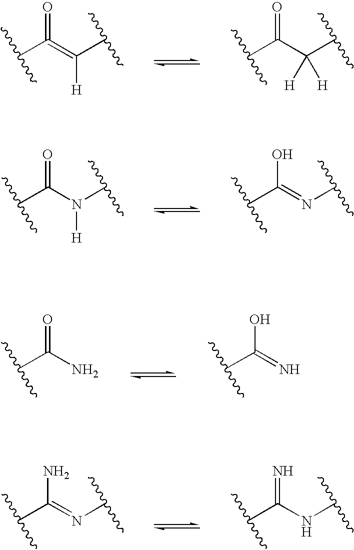

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

The term “catalytic group” refers to a chemical functional group that assists catalysis by acting to lower the activation barrier to reaction.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons (except in those instances where macromolecular substituents are clearly intended, e.g., polypeptides, polysaccharides, polyethylene glycols, DNA, RNA and the like).

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “hydrocarbon” as used herein, alone or in combination, refers to a compound or chemical group containing only carbon and hydrogen atoms.

The terms “heteroatom” or “hetero” as used herein, alone or in combination, refer to an atom other than carbon or hydrogen. Heteroatoms are may be independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “alkenyl” as used herein, alone or in combination, refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂-C₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkynyl” as used herein, alone or in combination, refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “heteroalkyl”, “heteroalkenyl” and “heteroalkynyl” as used herein, alone or in combination, refer to optionally substituted alkyl, alkenyl and alkynyl structures respectively, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof), or heteroatomic group such as though not limited to —O—O—, —S—S—, —O—S—, —S—O—, ═N—N═, —N═N—, —N═N—NH—, —P(O)₂—, —O—P(O)₂—, —P(O)₂—O—, —S(O)—, —S(O)₂—, —SnH₂— and the like.

The terms “haloalkyl”, “haloalkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to an optionally substituted, saturated, hydrocarbon monoradical ring, containing from three to about fifteen ring carbon atoms or from three to about ten ring carbon atoms, though may include additional, non-ring carbon atoms as substituents (e.g. methylcyclopropyl). Whenever it appears herein, a numerical range such as “C₃-C₆ cycloalkyl” or “C₃₋₆ cycloalkyl”, means that the cycloalkyl group may consist of 3 carbon atoms, 4 carbon atoms, 5 carbon atoms or 6 carbon atoms, i.e., is cyclopropyl, cyclobutyl, cyclopentyl or cyclohepty, although the present definition also covers the occurrence of the term “cycloalkyl” where no numerical range is designated. The term includes fused, non-fused, bridged and spiro radicals. A fused cycloalkyl may contain from two to four fused rings where the ring of attachment is a cycloalkyl ring, and the other individual rings may be alicyclic, heterocyclic, aromatic, heteroaromatic or any combination thereof. Examples include, but are not limited to cyclopropyl, cyclopentyl, cyclohexyl, decalinyl, and bicyclo[2.2.1]heptyl and adamantyl ring systems. Illustrative examples include, but are not limited to the following moieties:

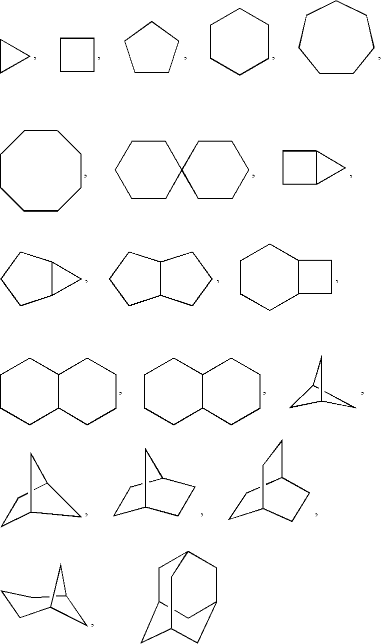

and the like.

The term “cycloalkenyl” as used herein, alone or in combination, refers to an optionally substituted hydrocarbon non-aromatic, monoradical ring, having one or more carbon-carbon double-bonds and from three to about twenty ring carbon atoms, three to about twelve ring carbon atoms, or from three to about ten ring carbon atoms. The term includes fused, non-fused, bridged and spiro radicals. A fused cycloalkenyl may contain from two to four fused rings where the ring of attachment is a cycloalkenyl ring, and the other individual rings may be alicyclic, heterocyclic, aromatic, heteroaromatic or any combination thereof. Fused ring systems may be fused across a bond that is a carbon-carbon single bond or a carbon-carbon double bond. Examples of cycloalkenyls include, but are not limited to cyclohexenyl, cyclopentadienyl and bicyclo[2.2.1]hept-2-ene ring systems. Illustrative examples include, but are not limited to the following moieties:

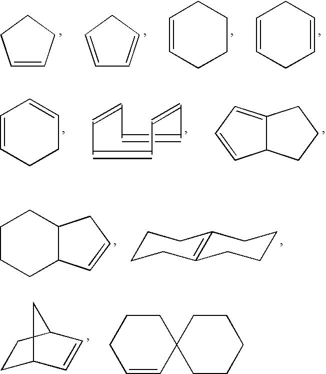

and the like.

The term “heterocycloalkyl” as used herein, alone or in combination, refer to optionally substituted, saturated, partially unsaturated or fully unsaturated nonaromatic ring monoradicals containing from three to about twenty ring atoms, where one or more of the ring atoms are an atom other than carbon, independently selected from among oxygen, nitrogen, sulfur, phosphorous, silicon, selenium and tin but are not limited to these atoms. In embodiments in which two or more heteroatoms are present in the ring, the two or more heteroatoms can be the same as each another, or some or all of the two or more heteroatoms can each be different from the others. The terms include fused, non-fused, bridged and spiro radicals. A fused non-aromatic heterocyclic radical may contain from two to four fused rings where the attaching ring is a non-aromatic heterocycle, and the other individual rings may be alicyclic, heterocyclic, aromatic, heteroaromatic or any combination thereof. Fused ring systems may be fused across a single bond or a double bond, as well as across bonds that are carbon-carbon, carbon-hetero atom or hetero atom-hetero atom. The terms also include radicals having from three to about twelve skeletal ring atoms, as well as those having from three to about ten skeletal ring atoms. Attachment of a non-aromatic heterocyclic subunit to its parent molecule can be via a heteroatom or a carbon atom. Likewise, additional substitution can be via a heteroatom or a carbon atom. As a non-limiting example, an imidazolidine non-aromatic heterocycle may be attached to a parent molecule via either of its N atoms (imidazolidin-1-yl or imidazolidin-3-yl) or any of its carbon atoms (imidazolidin-2-yl, imidazolidin-4-yl or imidazolidin-5-yl). In certain embodiments, non-aromatic heterocycles contain one or more carbonyl or thiocarbonyl groups such as, for example, oxo- and thio-containing groups. Examples include, but are not limited to pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thiepanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Illustrative examples of heterocycloalkyl groups, also referred to as non-aromatic heterocycles, include:

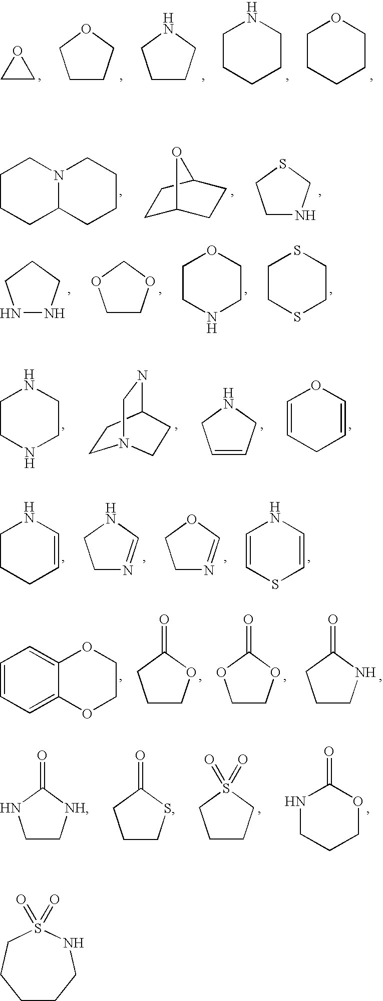

and the like.

The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized π-electron system containing 4n+2π electrons, where n is an integer. Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an optionally substituted aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

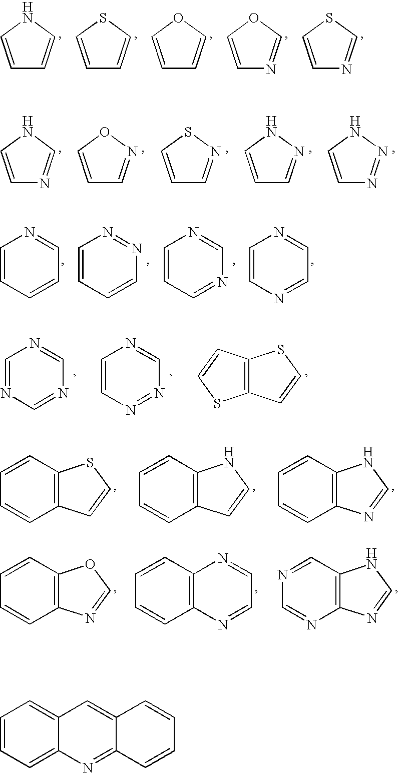

and the like.

The terms “halogen”, “halo” or “halide” as used herein, alone or in combination refer to fluoro, chloro, bromo and iodo.

The term “hydroxy” as used herein, alone or in combination, refers to the monoradical —OH.

The term “cyano” as used herein, alone or in combination, refers to the monoradical —CN.

The term “nitro” as used herein, alone or in combination, refers to the monoradical —NO₂.

The term “oxy” as used herein, alone or in combination, refers to the diradical —O—.

The term “oxo” as used herein, alone or in combination, refers to the diradical ═O.

The term “carbonyl” as used herein, alone or in combination, refers to the diradical —C(═O)—, which may also be written as —C(O)—.

The terms “carboxy” or “carboxyl” as used herein, alone or in combination, refer to the moiety —C(O)OH, which may also be written as —COOH.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tert-butoxy and the like.

The term “sulfinyl” as used herein, alone or in combination, refers to the diradical —S(═O)—.

The term “sulfonyl” as used herein, alone or in combination, refers to the diradical —S(═O)₂—.

The terms “sulfonamide”, “sulfonamido” and “sulfonamidyl” as used herein, alone or in combination, refer to the diradical groups —S(═O)₂—NH— and —NH—S(═O)₂—.

The terms “sulfamide”, “sulfamido” and “sulfamidyl” as used herein, alone or in combination, refer to the diradical group —NH—S(═O)₂—NH—.

The term “reactant,” as used herein, refers to a nucleophile or electrophile used to create covalent linkages.

The terms “group designed to improve water solubility”, “water solubilizing group” and the like as used herein, alone or in combination, refer to chemical groups and/or substituents that increase the solubility in water of the compounds described herein to the corresponding compound lacking the substituent (i.e. wherein the substituent is hydrogen). Non-limiting examples of water solubilizing groups include substituted or unsubstituted ethyleneoxy or polyethyleneoxy derivatives, such as:

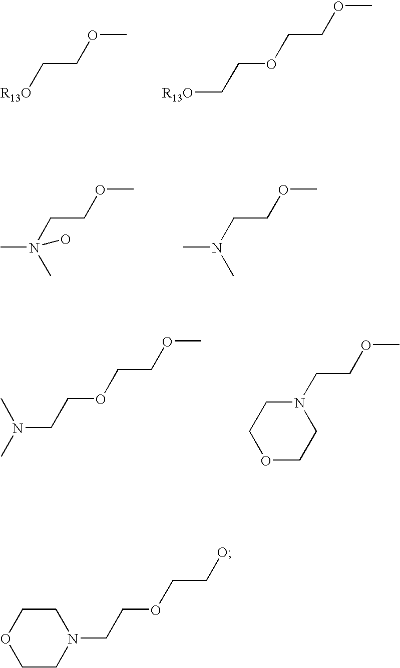

where R₁₃ is hydrogen, a sulfate salt, a phosphate salt, an extended PEG moiety and the like. Further non-limiting examples of water solubilizing groups include C₁-C₆ alkoxycarbonyl (e.g. —COOMe), cyano, halo, hydroxy, mercapto, oxo (═O), carboxy (—COOH), nitro, pyrrolidinyl, piperidinyl, imidazolidinyl, imidazolinyl, piperazinyl, morpholinyl, thiomorpholinyl and —NR_{f}R_{g}, wherein R_{f} and R_{g} may be the same or different and are independently chosen from hydrogen, C₁-C₆ alkyl, C₃-C₆ cycloalkyl, and the corresponding tertiary amine N-oxides. Further non-limiting examples of water solubilizing groups include:

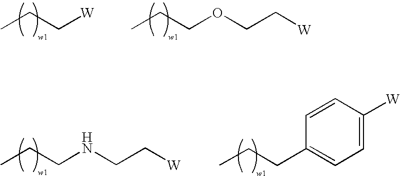

Where W is selected from:

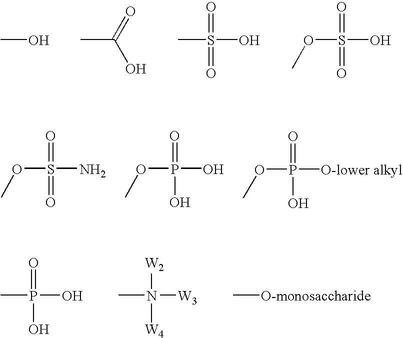

Where W₁ is 0, 1, 2 or 3; W₂ and W₃ are each independently hydrogen or methyl or, when taken together, W₂ and W₃ form a five or six membered ring that optionally contains an oxygen atom or a second nitrogen atom; and W₄ is an electron pair or an oxygen atom.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

Certain Pharmaceutical Terminology

The HDACs are a family including at least eighteen enzymes, grouped in three classes (Class I, II and III). Class I HDACs include, but are not limited to, HADCs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not interact with all three HDAC classes.

The term “HDAC modulator” as used herein refers to a compound that has the ability to modulate transcriptional activity.

The term “HDAC inhibitor” as used herein refers to a compound that has the ability to reduce transcriptional activity. As a result, this therapeutic class is able to block angiogenesis and cell cycling, and promote apoptosis and differentiation. By targeting these key components of tumor proliferation, HDAC inhibitors have the potential as anticancer agents. HDAC inhibitors both display targeted anticancer activity by itself and improve the efficacy of existing agents as well as other new targeted therapies.

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, the terms “cancer treatment” “cancer therapy” and the like encompasses treatments such as surgery, radiation therapy, administration of chemotherapeutic agents and combinations of any two or all of these methods. Combination treatments may occur sequentially or concurrently. Treatments(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

Examples of surgeries that may be used for cancer treatment include, but are not limited to radical prostatectomy, cryotherapy, mastectomy, lumpectomy, transurethral resection of the prostate, and the like.

Many chemotherapeutic agents are known and are discussed in greater detail herein. They may operate via a wide variety of modes of action such as, though not limited to, cytotoxic agents, antiproliferatives, targeting agents (such as monoclonal antibodies), and the like. The nature of a combination therapy involving administration of a chemotherapeutic agent will depend upon the type of agent being used.

The compounds described herein may be administered in combination with surgery, as an adjuvant, or as a neoadjuvant agent. The compounds described herein may be useful in instances where radiation and chemotherapy are indicated, to enhance the therapeutic benefit of these treatments, including induction chemotherapy, primary (neoadjuvant) chemotherapy, and both adjuvant radiation therapy and adjuvant chemotherapy. Radiation and chemotherapy frequently are indicated as adjuvants to surgery in the treatment of cancer. For example, radiation can be used both pre- and post-surgery as components of the treatment strategy for rectal carcinoma. The compounds described herein may be useful following surgery in the treatment of cancer in combination with radio- and/or chemotherapy.

Where combination treatments are contemplated, it is not intended that the compounds described herein be limited by the particular nature of the combination. For example, the compounds described herein may be administered in combination as simple mixtures as well as a chemical hybrids. An example of the latter is where the compound is covalently linked to a targeting carrier or to an active pharmaceutical. Covalent binding can be accomplished in many ways, such as, though not limited to, the use of a commercially available cross-linking compound.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

As used herein, the terms “co-administration”, “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “carrier” as used herein, refers to relatively nontoxic chemical compounds or agents that facilitate the incorporation of a compound into cells or tissues.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable derivative or prodrug” as used herein, refers to any pharmaceutically acceptable salt, ester, salt of an ester or other derivative of a compound of Formula I, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. (See for example Berge et al., J. Pharm. Sci. 1977, 66, 1-19.) Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄OH⁻, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “metabolite,” as used herein, refers to a derivative of a compound which is formed when the compound is metabolized.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Compounds

Described herein are compounds of Formula I:

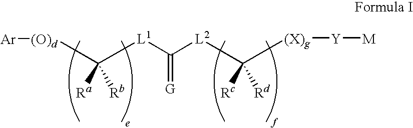

or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group

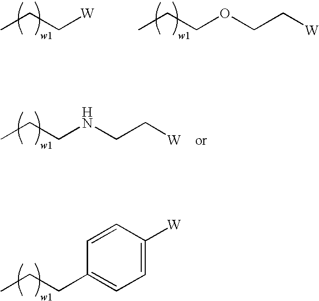

where W is selected from:

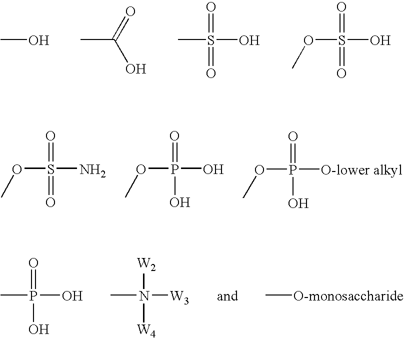

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is:

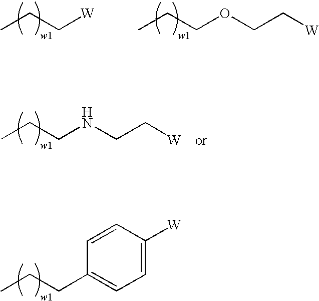

where W is selected from:

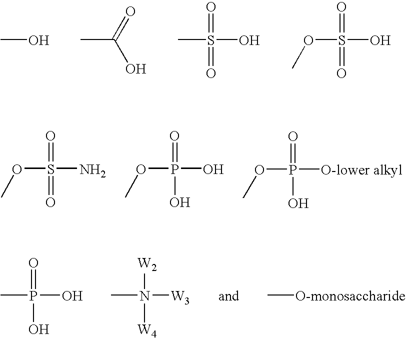

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂;

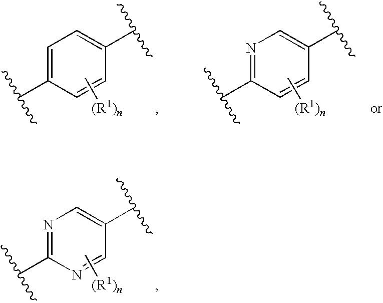

X is
wherein each R¹ is independently halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and L-heteroaryl, wherein L is a bond, —C(O)—, —S(O), and —S(O)₂,
n is 0, 1, 2.3 or 4, and the water solubilizing grout, is

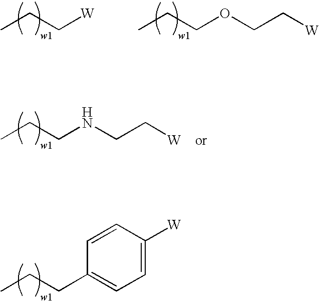

where W is selected from:

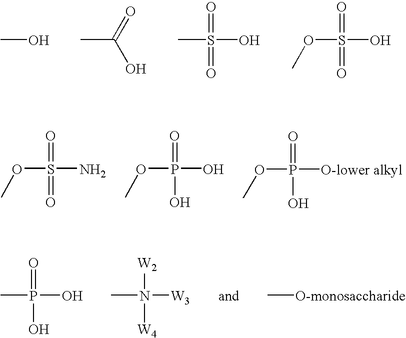

where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from

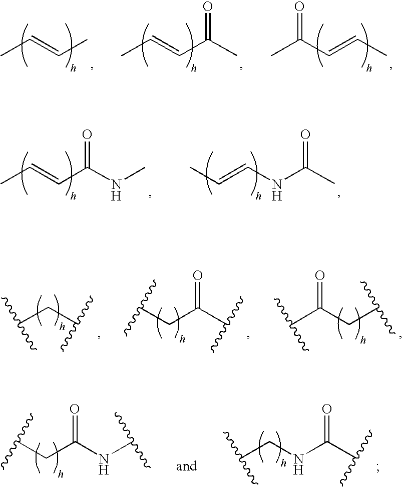

wherein h is 0, 1, 2, 3 or 4;
M is selected from

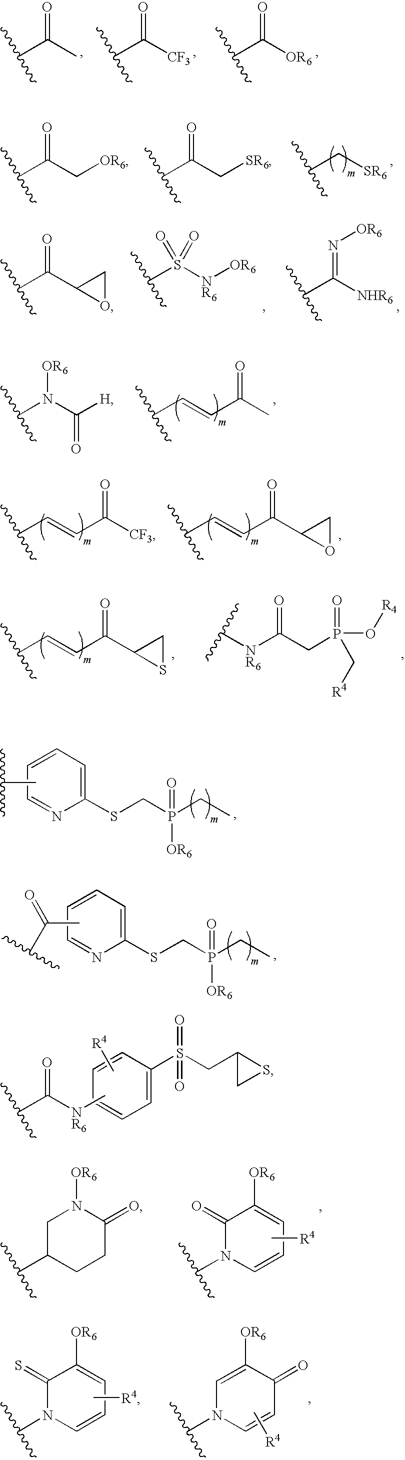

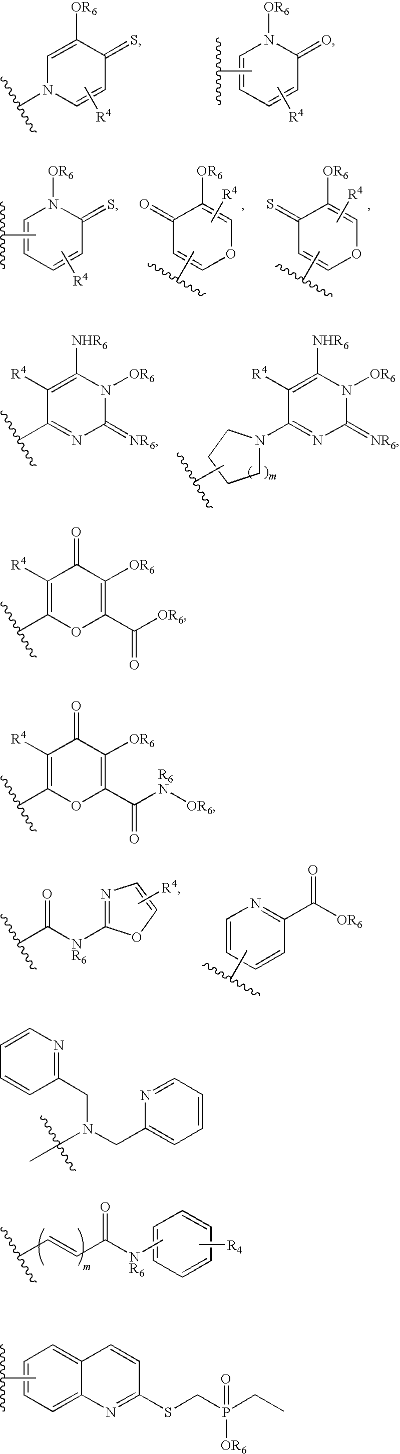

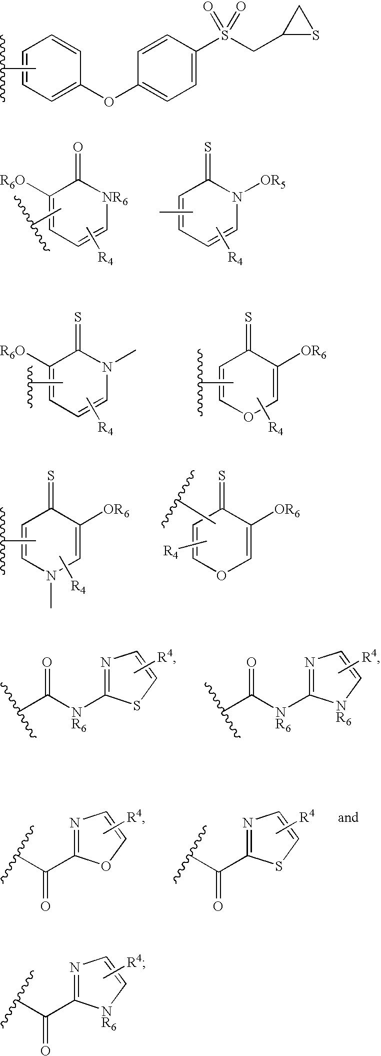

wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is:

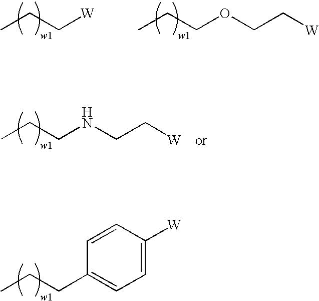

where W is selected from:

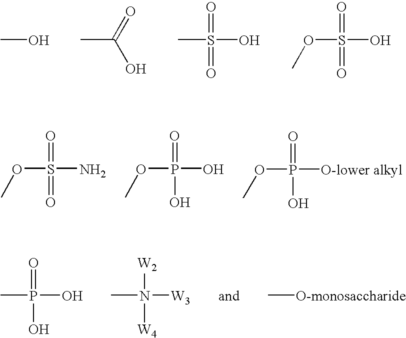

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R⁶ is hydrogen, lower alkyl, lower acyl, lower alkoxycarbonyl, carbamoyl, —PO₃H₂, and —SO₃H; and
m=0 or 1 or 2,
provided that when g is 1 and Y is amidomethyl or amidoethyl, then Y is not carboxyl or ethoxycarbonyl;
when g is 1 and Y is a bond, then M is not carboxyl; and
that the compound is not:

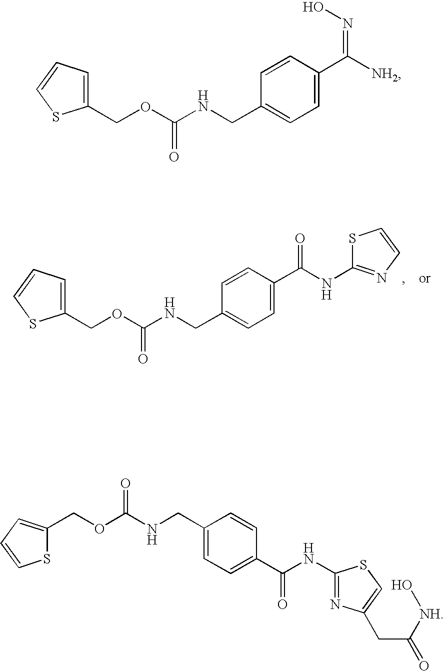

Described herein are compounds of Formula IA, where the substituents are as defined herein:

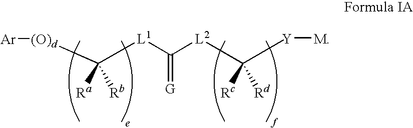

Described herein are compounds of Formula IB, where the substituents are as defined herein:

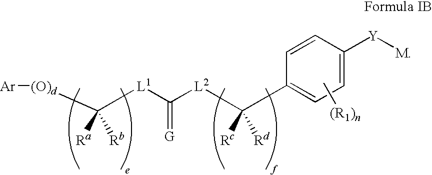

Described herein are compounds of Formula IC, where the substituents are as defined herein:

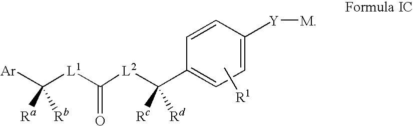

Described herein are compounds of Formula ID, where the substituents are as defined herein:

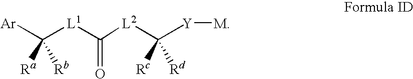

Described herein are compounds of Formula IE, where the substituents are as defined herein:

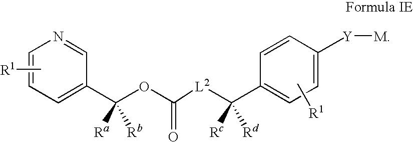

Described herein are compounds of Formula IF, where the substituents are as defined herein:

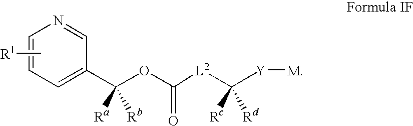

Described herein are compounds of Formula II, where the substituents are as defined herein:

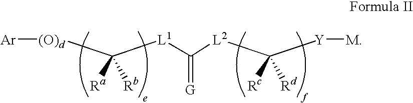

Described herein are compounds of Formula III, where the substituents are as defined herein:

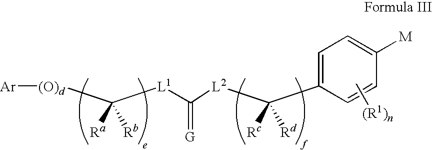

Described herein are compounds of Formula IV, where the substituents are as defined herein:

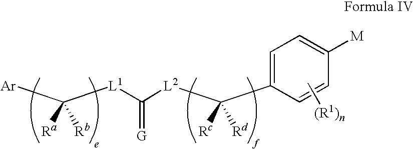

Described herein are compounds of Formula V, where the substituents are as defined herein:

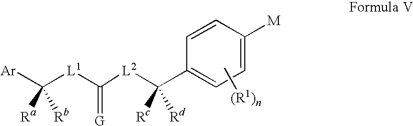

Described herein are compounds of Formula VI, where the substituents are as defined herein:

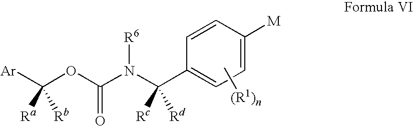

Described herein are compounds of Formula VII, where the substituents are as defined herein:

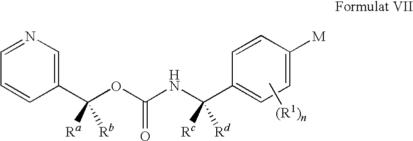

Described herein are compounds of Formula VIII, where the substituents are as defined herein:

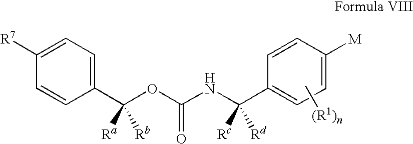

In some embodiments, Ar is an C₅-C₁₅ aryl or C₅-C₁₅ heteroaryl group, wherein said aryl or heteroaryl groups are optionally substituted with 1-3 substituents selected from halogen, —CN, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂. In some embodiments, the optional substituent is further substituted with a substituent selected from halogen, hydroxy, amino, carboxy, C₁-C₄ alkyl, C₁-C₄ alkoxy, C₁-C₄ aminoalkyl, C₁-C₄ alkylamino, C₁-C₄ alkylthio, haloalkyl, C₁-C₄ perfluoroalkyl, C₁-C₄ perfluoroalkoxy and C₁-C₄ alkoxycarbonyl. In some embodiments, the aryl or heteroaryl groups are optionally substituted with 1-3 substituents each independently selected from halogen, hydroxy, amino, carboxy, C₁-C₄ alkyl, C₁-C₄ alkoxy, C₁-C₄ aminoalkyl, C₁-C₄ alkylamino, C₁-C₄ alkylthio, C₁-C₄ haloalkyl, C₁-C₄ perfluoroalkyl, perfluoroalkoxy and C₁-C₄ alkoxycarbonyl. In some embodiments, these optional substituents are not further substituted. In some embodiments Ar is unsubstituted. In some embodiments, the aryl or heteroaryl group is substituted with one substituent. In other embodiments, the aryl or heteroaryl group is substituted with two substituents. In some embodiments, the aryl or heteroaryl group is substituted with 1-2 halogens. In some embodiments, the halogen is fluorine. In some embodiments, Ar is substituted with a water solubilizing group.

In some embodiments, the water solubilizing group on Ar is the water solubilizing group is

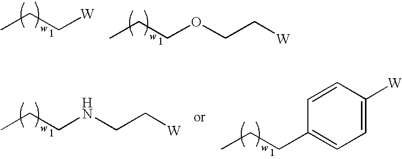

where W is selected from

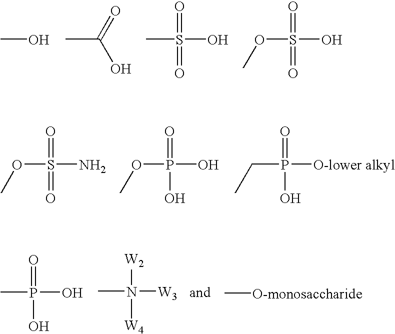

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom.

In some embodiments, Ar is a heteroaryl group. In some embodiments, Ar is a heteroaryl group selected from furanyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, triazolyl, triazinyl, or thiadiazolyl group. In some embodiments, Ar is a nitrogen-containing heteroaryl group.

In some embodiments, Ar is:

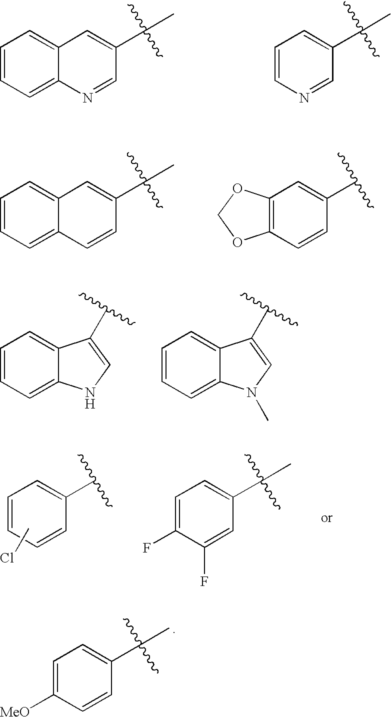

In some embodiments, Ar is

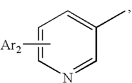

wherein Ar₂ is:

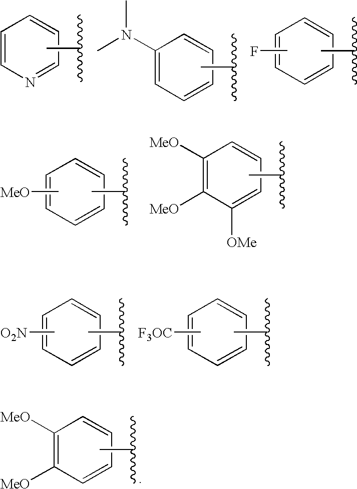

In some embodiments, Ar is:

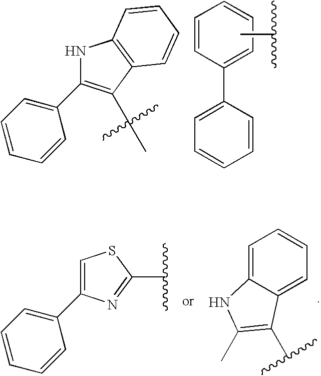

In some embodiments, d is 0. In other embodiments, d is 1.

In some embodiments, e and f combined is 2. In other embodiments, both e and f are 1.

In some embodiments, R^{a}, R^{b}, R^{c} and R^{d} are each independently hydrogen, halogen, —CN, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-alkoxy, -L-alkylamine, -L-dialkylamine, -L-aryl, and -L-heteroaryl, wherein L is a bond, —C(O)—, —S(O), and —S(O)₂. In some embodiments, R^{a}, R^{b}, R^{c} and R^{d} are each independently hydrogen, carboxy, C₁-C₄ alkyl group, C₂-C₅ alkenyl, C₂-C₅alkynyl, C₁-C₄haloalkyl, C₁-C₄ heteroalkyl, C₁-C₄ alkoxy, C₁-C₄ thioalkoxy, C₁-C₄ thioalkyl, or C₁-C₄ alkoxycarbonyl. In some embodiments, R^{a}, R^{b}, R^{c} and R^{d} are not further substituted. In some embodiments, R^{a}, R^{b}, R^{c} and R^{d} are each independently hydrogen, C₁-C₄ alkyl, or a water solubilizing group wherein the water solubilizing group is

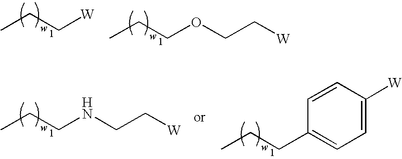

where W is selected from

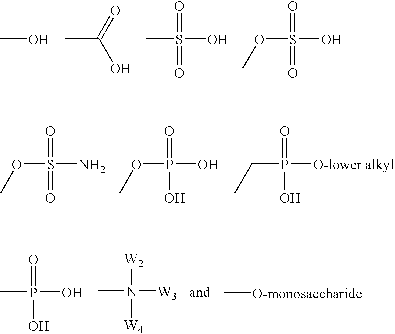

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom.

In some embodiments, three of R^{a}, R^{b}, R^{c} and R^{d} are hydrogen and the other is selected from hydrogen, alkyl, substituted alkyl, heteroalkyl, alkoxy, aryl, heteroaryl, carboxyalkyl, aminoalkyl, hydroxyalkyl and a water solubilizing group. In some embodiments, three of R^{a}, R^{b}, R^{c} and R^{d} are hydrogen and the other is an optionally substituted one to three carbon alkyl group. In some embodiments, R^{a}, R^{b}, R^{c} and R^{d} are each hydrogen. In other embodiments, at least one of R^{a}, R^{b}, R^{c}, and R^{d} is not hydrogen.

In some embodiments, G is O. In other embodiments, G is S. In still other embodiments, G is NR⁵, where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, or carboxyalkyl. In some embodiments, R⁵ is a substituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, or carboxyalkyl, wherein the substitution is selected from halogen, —CN, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂. In some embodiments, R⁵ is a substituted group and the substituent is selected from hydrogen, carboxy, and unsubstituted C₁-Cₐ alkyl group, C₂-C₅ alkenyl, C₂-C₅ alkynyl, C₁-C₄ haloalkyl, C₁-C₄ heteroalkyl, C₁-C₄ alkoxy, thioalkoxy, C₁-C₄ thioalkyl, or C₁-C₄ alkoxycarbonyl. In some embodiments, R⁵ is hydrogen or C₁-C₄ alkyl. In some embodiments, R⁵ is a prodrug. In some embodiments, R⁵ is C₁-C₄ alkyl, C₂-C₅ alkenyl, C₂-C₅ alkynyl, C₁-C₄ alkoxy, C₂-C₆ hydroxyalkyl, C₂-C₆ aminoalkyl, C₂-C₆ alkylamino, C₂-C₆ mercaptoalkyl, C₂-C₆ perfluoroalkyl, C₁-C₄ perfluoroalkoxy, C₂-C₆ carboxyalkyl, C₂-C₆ alkoxycarbonylalkyl or C₂-C₆ alkoxycarbonyloxyalkyl. In some embodiments, R⁵ is hydrogen, C₁-C₄ alkyl or a watersolubilizing group wherein the water solubilizing group is

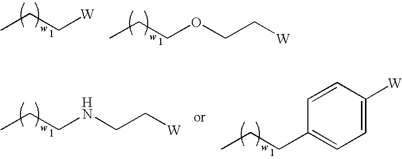

where W is selected from

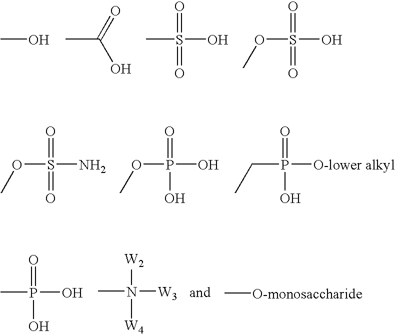

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom.

In some embodiments, L¹ and L² are each independently —O—, —N(R³)—, —ON(R³)—, or —N(R³)O—; where R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, or carboxyalkyl group. In some embodiments, R³ is hydrogen, C₁-C₄ alkyl, or a water solubilizing group wherein the water solubilizing group is

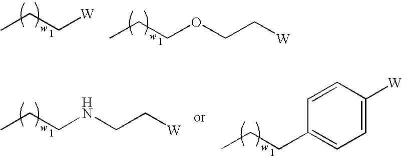

where W is selected from

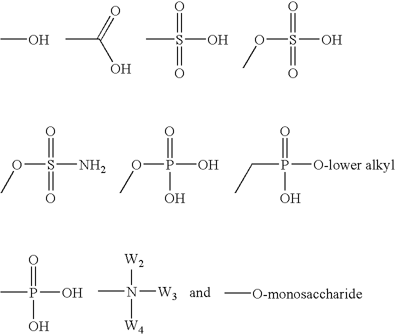

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom.

In some embodiments, one of L¹ and L² is —O— and one is —N(R³)—. In some embodiments, R³ is hydrogen or a substituted or unsubstituted C₁-C₄ alkyl group, C₂-C₅ alkenyl, C₂-C₅ alkynyl, C₁-C₄ haloalkyl, C₁-C₄ heteroalkyl, C₁-C₄ alkoxy, C₁-C₄ thioalkoxy, C₁-C₄ thioalkyl, or C₁-C₄ alkoxycarbonyl. In some embodiments, R³ is hydrogen or an unsubstituted C₁-C₄ alkyl group, C₂-C₅ alkenyl, C₂-C₅ alkynyl, C₁-C₄ haloalkyl, C₁-C₄ heteroalkyl, C₁-C₄ alkoxy, C₁-C₄ thioalkoxy, C₁-C₄ thioalkyl, or C₁-C₄ alkoxycarbonyl. In some embodiments, L¹ and L² are each independently —O—, —N(R³)—, —ON(R³)—, —N(R³)O—; wherein R³ is hydrogen, C₁-C₄ alkyl, C₂-C₅ alkenyl, C₂-C₅ alkynyl, C₁-C₄ alkoxy, C₂-C₆ hydroxyalkyl, C₂, —C₆ aminoalkyl, C₂-C₆ alkylamino, C₂-C₆ mercaptoalkyl, C₂-C₆ perfluoroalkyl, C₁-C₄ perfluoroalkoxy, C₂-C₆ carboxyalkyl, C₂-C₆ alkoxycarbonylalkyl or C₂-C₆ alkoxycarbonyloxyalkyl.

In some embodiments, L¹ and L² are independently —O— or —N(R³)—, wherein R³ is hydrogen or a water solubilizing group. In some embodiments, L₁ is NH. In some embodiments, L₂ is NH. In some embodiments L₁ and L₂ are both NH.

In some embodiments, X is

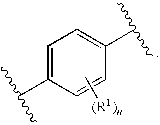

In some embodiments, X is

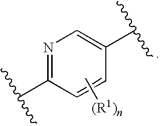

In some embodiments, X is

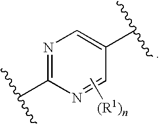

In yet other embodiments, X is

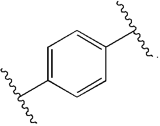

In yet other embodiments, X is

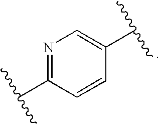

In still other embodiments, X is

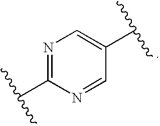

In some embodiments, Y is

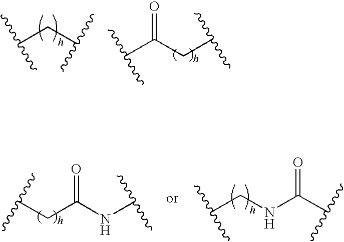

where h is 0 or 1. In some embodiments, Y is

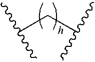

where h is 1.

In some embodiments, Y is

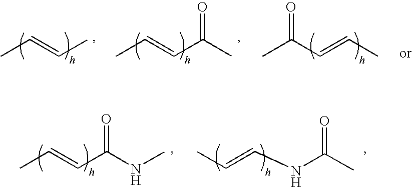

where h is 0 or 1. In some embodiments, Y is

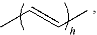

where h is 1. In some embodiments, Y is

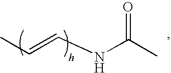

where h is 0 or 1.

In some embodiments, Y is selected from

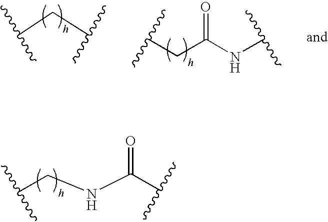

wherein h is 0, 1, or 2.

In some embodiments, M is selected from

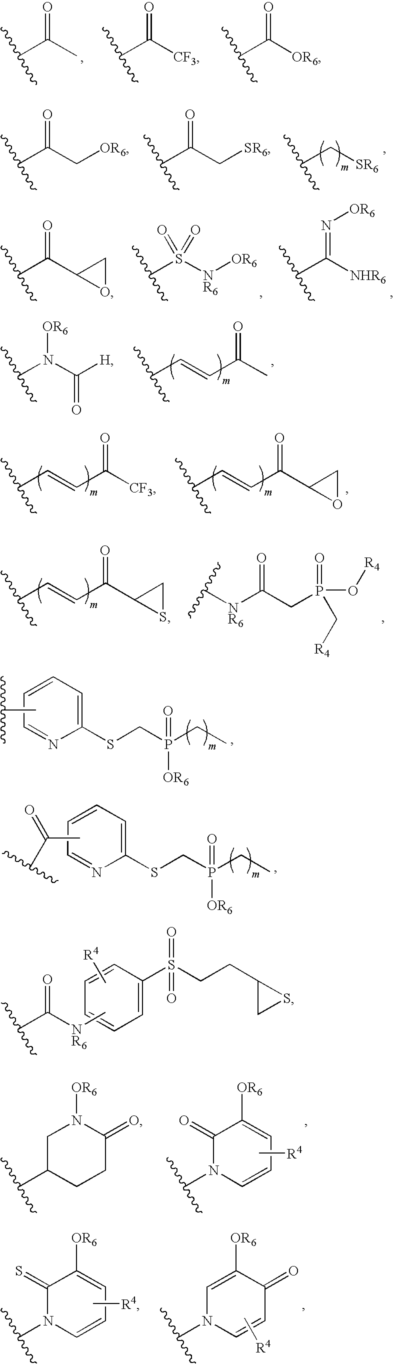

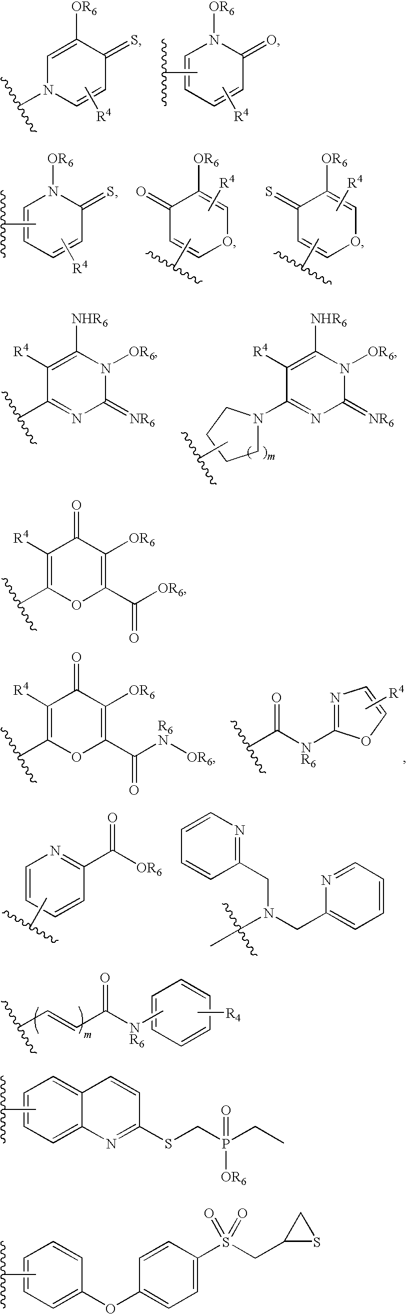

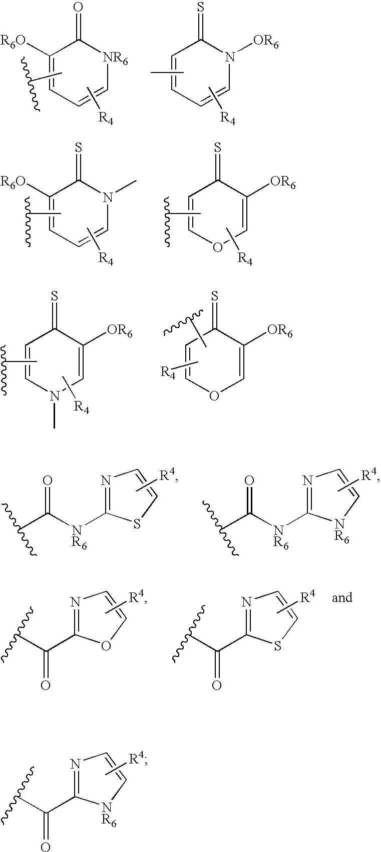

In some embodiments, R⁶ is hydrogen, lower alkyl, lower acyl, lower alkoxycarbonyl, carbamoyl, —PO₃H₂, and —SO₃H. In some embodiments, R⁶ is hydrogen, acetyl, carbamoyl, —PO₃H₂, and —SO₃H. In other embodiments, R⁶ is acetyl. In yet other embodiments, R⁶ is hydrogen.

In some embodiments, M is selected from

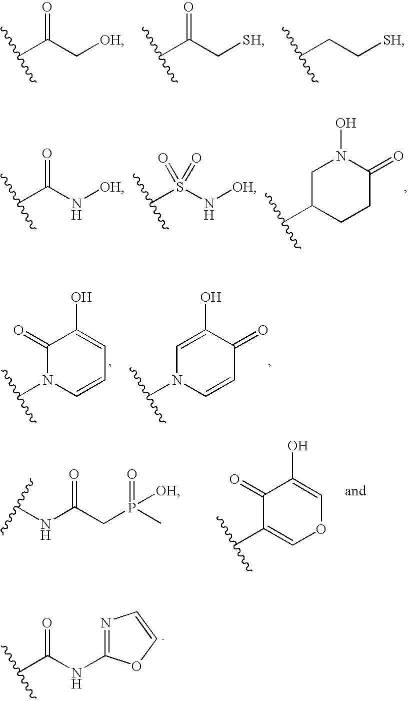

In some embodiments, M is selected from

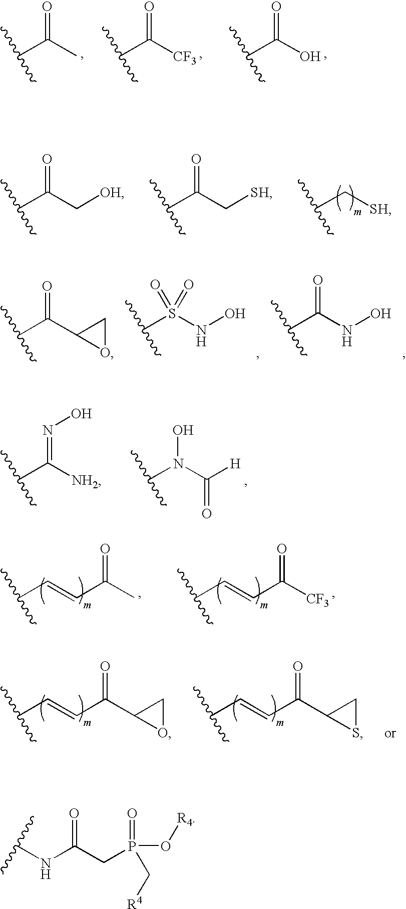

In some embodiments, M is selected from

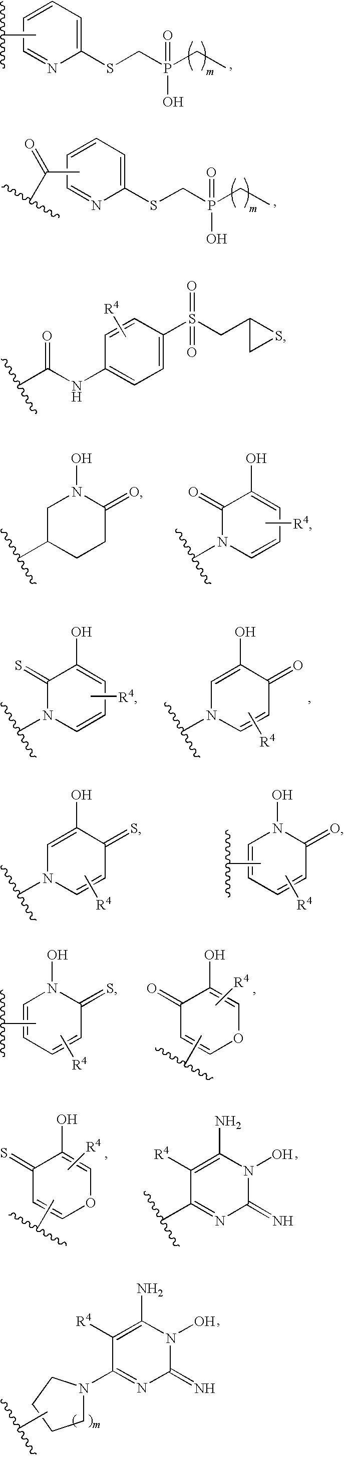

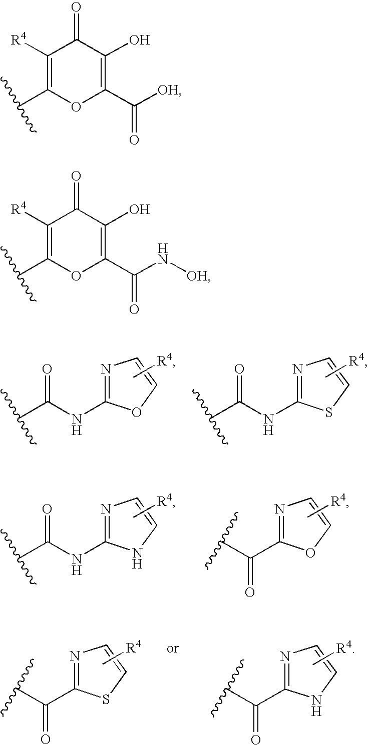

In some embodiments, n is 0. In other embodiments, n is 1.

In some embodiments, M is:

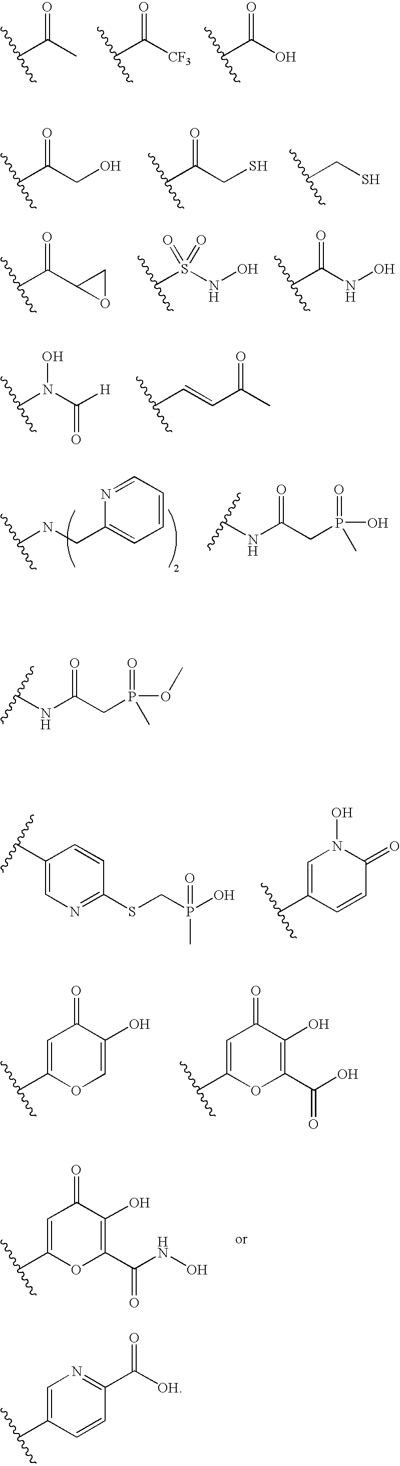

In some embodiments, M is

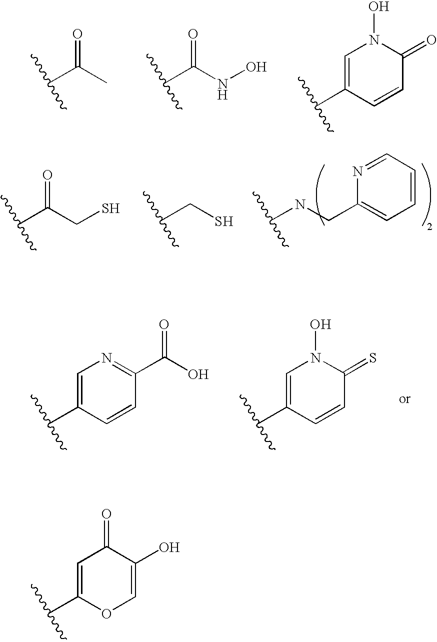

In some embodiments, (X)_{g}—Y-M is:

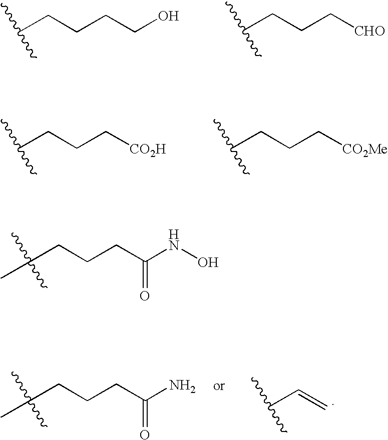

In some embodiments, (X)_{g}—Y-M is C₁-C₆ alkyl, C(O)C₁-C₄ alkyl, C₁-C₆ alkyl-OH, C₁-C₆ alkyl-C(O)H, C₁-C₆ alkyl-C(O)OH, C₁-C₆ alkyl-C(O)O—C₁-C₄ alkyl, C₁-C₆ alkyl-C(O)NHOH, C₁-C₆ alkyl-C(O)NH₂, or C₁-C₆ alkylene.

In some embodiments, (X)_{g}—Y-M is:

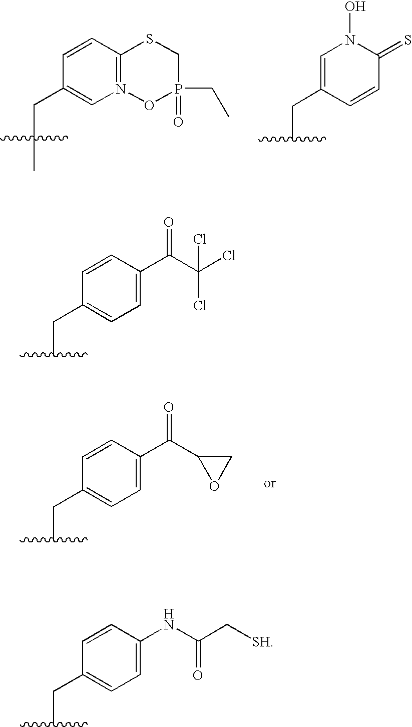

In some embodiments, R⁴ is hydrogen, halogen, —CN, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂; and m=0 or 1 or 2. In some embodiments, R⁴ is hydrogen, halogen, hydroxy, or a substituted or unsubstituted group selected from amino, carboxy, C₁-C₄ alkyl, C₂-C₅ alkenyl, C₂-C₅ alkynyl, C₁-C₄ alkoxy, C₁-C₄ aminoalkyl, C₁-C₄ alkylamino, C₁-C₄ alkylthio, perfluoroalkyl, C₁-C₄ perfluoroalkoxy or C₁-C₄ alkoxycarbonyl. In further embodiments, R⁴ is not further substituted. In some embodiments, R⁴ is hydrogen or C₁-C₄ alkyl. In some embodiments R⁴ is hydroxy. In some embodiments, R⁴ is hydrogen, C₁-C₃ alkyl, or a water solubilizing group, wherein the water solubilizing group is

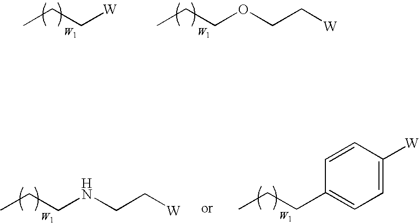

where W is selected from

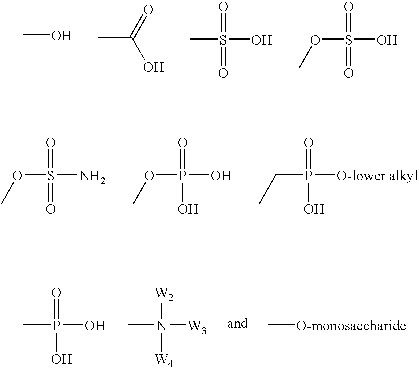

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom.

In some embodiments, R⁷ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, n is 0, 1, 2, 3 or 4 and the water solubilizing group is

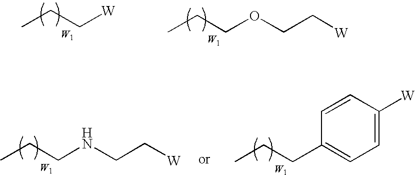

where W is selected from

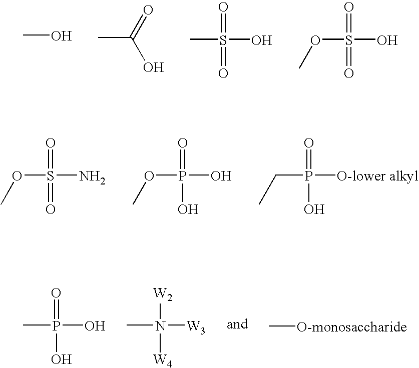

where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom. In some embodiments, R⁷ is hydrogen, halogen, hydroxy, or a substituted or unsubstituted group selected from amino, carboxy, C₁-C₄ alkyl, C₂-C₅ alkenyl, C₂-C₅ alkynyl, C₁-C₄ alkoxy, C₁-C₄ aminoalkyl, C₁-C₄ alkylamino, C₁-C₄ alkylthio, C₁-C₄ perfluoroalkyl, C₁-C₄ perfluoroalkoxy or C₁-C₄ alkoxycarbonyl. In some embodiments, R⁷ is not further substituted. In some embodiments, R⁷ is hydrogen or C₁-C₄ alkyl. In some embodiments R⁷ is halogen, hydroxy or an amino group. In some embodiments, R⁷ is a substituted or unsubstituted aryl or heteroaryl group.

In various embodiments, the water solubilizing group is selected from cyano, halo, hydroxy, mercapto, oxo, carboxy, nitro, substituted or unsubstituted pyrrolidinyl, substituted or unsubstituted piperidinyl, substituted or unsubstituted imidazolidinyl, substituted or unsubstituted imidazolinyl, substituted or unsubstituted piperazinyl, substituted or unsubstituted morpholinyl, substituted or unsubstituted thiomorpholinyl substituted or unsubstituted ethyleneoxide, substituted or unsubstituted polyethyleneoxide, C₁-C₆ alkoxycarbonyl, and —NR_{f}R_{g}, wherein R_{f} and R_{g} may be the same or different and are independently chosen from hydrogen, C₁-C₆ alkyl and C₃-C₆ cycloalkyl. In some embodiments, the water solubilizing group is

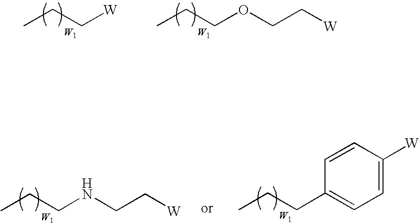

where W is selected from:

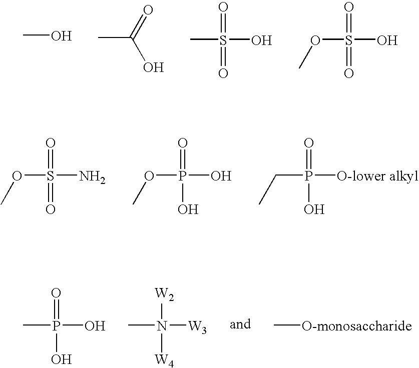

where W₁ is 0 or 1 or 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom. In some embodiments, the water solubilizing group is

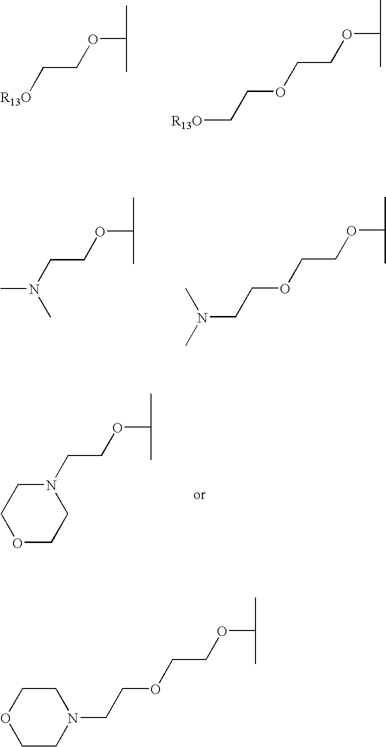

where R₁₃ is hydrogen, C₁-C₆ alkyl, a sulfate salt or a phosphate salt.

In some embodiments, g is 0. In some embodiments g is 0; Y is In some embodiments, g is 0. In some embodiments g is 0; Y is

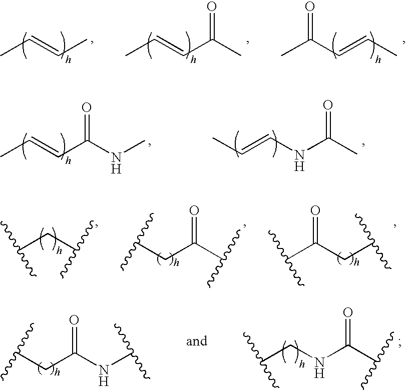

wherein h is 0, 1, 2, 3 or 4;
where h is 0, 1, 2, 3 or 4; and M is:

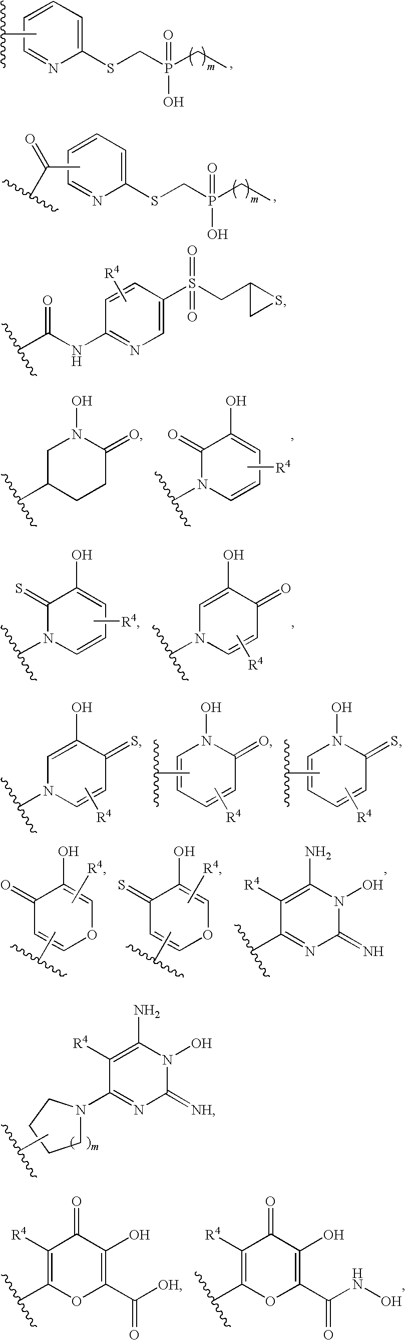

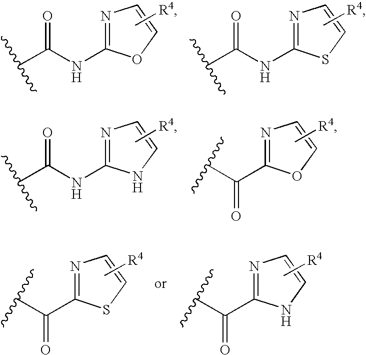

where R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂; and m=0, 1 or 2. In some embodiments, R⁴ is hydrogen or C₁-C₄ alkyl.
   In some embodiments, g is 1. In some embodiments, g is 1; Y is

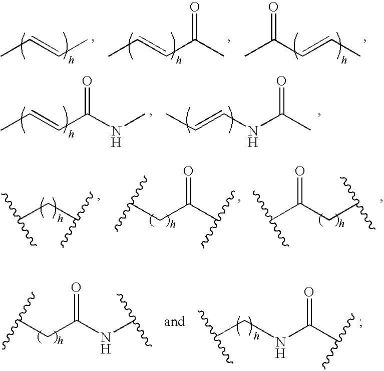

wherein h is 0, 1, 2, 3 or 4;
where h is 0, 1, 2, 3 or 4; and M is:

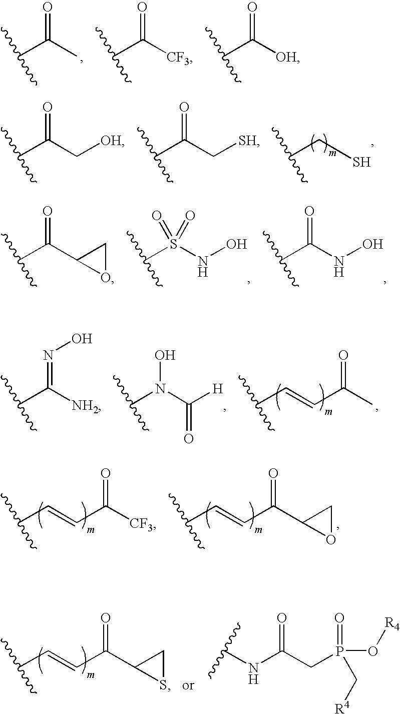

where R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂; and m=0 or 1 or 2. In some embodiments, R⁴ is hydrogen or C₁-C₄ alkyl.

In some embodiments, g is 1, X is

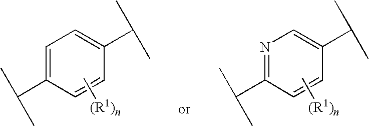

and each R¹ is independently halogen, hydroxy, or a substituted or unsubstituted group selected from amino, carboxy, C₁-C₄ alkyl, C₂-C₅ alkenyl, C₂-C₅ alkynyl, C₁-Cₐ alkoxy, C₁-Cₐ aminoalkyl, C₁-C₄ alkylamino, C₁-C₄ alkylthio, C₁-Cₐ perfluoroalkyl, C₁-C₄ perfluoroalkoxy or C₁-C₄ alkoxycarbonyl; and n is 1 or 2. In some embodiments, at least one R₁ is in the ortho position.

In some embodiments, f is 1 or 2; g is 0; and R^{a}, R^{b}, R^{c} and R^{d} are each hydrogen. In other embodiments, f is 1 or 2; g is 1; and R^{a}, R^{b}, R^{c} and R^{d} are each hydrogen.

In some embodiments, d is 0; e is 1; f is 1; R^{a}, R^{b}, R^{c} and R^{d} are each independently hydrogen, carboxy, C₁-C₄ alkyl, C₂-C₅ alkenyl, C₂-C₅ alkynyl, C₁-C₄ haloalkyl, C₁-C₄ heteroalkyl, alkoxy, C₁-C₄ thioalkoxy, C₁-C₄ thioallyl, or C₁-C₄ alkoxycarbonyl, wherein at least one of R^{a}, R^{b}, R^{c} and R^{d} is not hydrogen; L¹ and L² are each independently —O— or —N(R³)—, wherein R³ is hydrogen, C₁-C₄ alkyl, C₁-C₄ alkoxy, C₂-C₆ hydroxyalkyl, C₂-C₆ aminoalkyl, C₂-C₆ alkylamino, C₂-C₆ mercaptoalkyl, C₂-C₆ perfluoroalkyl, C₁-C₄ perfluoroalkoxy, C₂-C₆ carboxyalkyl, C₂-C₆ alkoxycarbonylalkyl or C₂-C₆ alkoxycarbonyloxyalkyl; and G is 0.

In a preferred embodiment, the invention provides for compounds of Formulas I-VIII and their pharmaceutically acceptable salts. In further or additional embodiments, the invention provides for compounds of Formulas I-VIII and their pharmaceutically acceptable solvates. In further or additional embodiments, the invention provides for compounds of Formulas I-VIII and their pharmaceutically acceptable polymorphs. In further or additional embodiments, the invention provides for compounds of Formulas I-VIII and their pharmaceutically acceptable esters. In further or additional embodiments, the invention provides for compounds of Formulas I-VIII and their pharmaceutically acceptable tautomers. In further or additional embodiments, the invention provides for compounds of Formulas I-VIII and their pharmaceutically acceptable prodrugs.

It should be understood that each of the above substituents or groups of substituents may be used in Formulas I-VIII.

Provided herein are pharmaceutical compositions comprising a compound of Formulas I-VIII or a pharmaceutically acceptable salt, prodrug, solvate, polymorph, tautomer or isomer thereof. In various embodiments, the pharmaceutical composition comprises at least one pharmaceutically acceptable carrier.

Provided herein are methods for treating a patient suffering from a histone deacetylase mediated disorder, comprising administering to said individual an effective amount of a composition comprising a compound of Formulas I-VIII or a pharmaceutically acceptable salt, prodrug, solvate, polymorph, tautomer or isomer thereof. In some embodiments, the compound of Formulas I-VIII is administered in combination with an additional cancer therapy. In some embodiments, the additional cancer therapy is selected from surgery, radiation therapy, and administration of at least one chemotherapeutic agent. In various embodiments, the administration of the compound of Formulas occurs after surgery. In other embodiments, the administration of the compound of Formulas I-VIII occurs before surgery. In some embodiments, the histone deacetylase mediated disorder is selected from the group consisting of inflammatory diseases, infections, autoimmune disorders, stroke, ischemia, cardiac disorder, neurological disorders, fibrogenetic disorders, proliferative disorders, hyperproliferative disorders, tumors, leukemias, neoplasms, cancers, carcinomas, metabolic diseases and malignant diseases. In other embodiments, the histone deacetylase mediated disorder is a hyperproliferative disease. In some embodiments, the histone deacetylase mediated disorder is cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell line cancer. In yet other embodiments, the histone deacetylase mediated disorder is a proliferative disease selected from psoriasis, restenosis, autoimmune disease, or atherosclerosis.

Provided herein are methods for degrading, inhibiting the growth of or killing cancer cells comprising contacting the cells with an amount of a composition effective to degrade, inhibit the growth of or kill cancer cells, the composition comprising a compound of Formulas I-VIII or a pharmaceutically acceptable salt, prodrug, solvate, polymorph, tautomer or isomer thereof. In some embodiments, the cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell line cancer. In some embodiments, the cancer cells comprise brain, breast, lung, ovarian, pancreatic, prostate, renal, or colorectal cancer cells.

Provided herein are methods of inhibiting tumor size increase, reducing the size of a tumor, reducing tumor proliferation or preventing tumor proliferation in an individual comprising administering to said individual an effective amount of a composition to inhibit tumor size increase, reduce the size of a tumor, reduce tumor proliferation or prevent tumor proliferation, the composition comprising a compound of Formulas I-VIII or a pharmaceutically acceptable salt, prodrug, solvate, polymorph, tautomer or isomer thereof. In some embodiments, the tumor occurs in the brain, breast, lung, ovaries, pancreas, prostate, kidney, colon or rectum. In some embodiments the compound of Formulas I-VIII is administered in combination with an additional cancer therapy including, but not limited to surgery, radiation therapy, and administration of at least one chemotherapeutic agent. In some embodiments, the composition is administered before surgery. In other embodiments, the composition is administered after surgery.

Compounds of Formulas I-VIII, pharmaceutically acceptable salts, pharmaceutically active metabolites, pharmaceutically acceptable prodrugs, and pharmaceutically acceptable solvates thereof, may modulate the activity of HDAC enzymes; and, as such, are useful for treating diseases or conditions in which aberrant HDAC enzyme activity contributes to the pathology and/or symptoms of a disease or condition.

Synthetic Procedures

In another aspect, methods for synthesizing the compounds described herein are provided. In some embodiments, the compounds described herein can be prepared by the methods described below. The procedures and examples below are intended to illustrate those methods. Neither the procedures nor the examples should be construed as limiting the invention in any way. Compounds described herein may also be synthesized using standard synthetic techniques known to those of skill in the art or using methods known in the art in combination with methods described herein. In additions, solvents, temperatures and other reaction conditions presented herein may vary according to the practice and knowledge of those of skill in the art.

The starting materials used for the synthesis of the compounds as described herein can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

The compounds described herein can be modified using various electrophiles or nucleophiles to form new functional groups or substituents. The table below entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

Examples of Covalent Linkages and Precursors Thereof

| Covalent Linkage | | |
|---|---|---|
| Product | Electrophile | Nucleophile |
| Carboxamides | Activated esters | Amines/anilines |
| Carboxamides | Acyl azides | Amines/anilines |
| Carboxamides | Acyl halides | Amines/anilines |
| Esters | Acyl halides | Alcohols/phenols |
| Esters | Acyl nitriles | Alcohols/phenols |
| Carboxamides | Acyl nitriles | Amines/anilines |
| Imines | Aldehydes | Amines/anilines |
| Hydrazones | Aldehydes or | Hydrazines |
| | ketones | |
| Oximes | Aldehydes or | Hydroxylamines |
| | ketones | |
| Alkyl amines | Alkyl halides | Amines/anilines |
| Esters | Alkyl halides | Carboxylic acids |
| Thioethers | Alkyl halides | Thiols |
| Ethers | Alkyl halides | Alcohols/phenols |
| Thioethers | Alkyl sulfonates | Thiols |
| Esters | Alkyl sulfonates | Carboxylic acids |
| Ethers | Alkyl sulfonates | Alcohols/phenols |
| Esters | Anhydrides | Alcohols/phenols |
| Carboxamides | Anhydrides | Amines/anilines |
| Thiophenols | Aryl halides | Thiols |
| Aryl amines | Aryl halides | Amines |
| Thioethers | Aziridines | Thiols |
| Boronate esters | Boronates | Glycols |
| Carboxamides | Carboxylic acids | Amines/anilines |
| Esters | Carboxylic acids | Alcohols |
| Hydrazines | Hydrazides | Carboxylic acids |
| N-Acylureas or | Carbodiimides | Carboxylic acids |
| Anhydrides | | |
| Esters | Diazoalkanes | Carboxylic acids |
| Thioethers | Epoxides | Thiols |
| Thioethers | Haloacetamides | Thiols |
| Aminotriazines | Halotriazines | Amines/anilines |
| Triazinyl ethers | Halotriazines | Alcohols/phenols |
| Amidines | Imido esters | Amines/anilines |
| Ureas | Isocyanates | Amines/anilines |
| Urethanes | Isocyanates | Alcohols/phenols |
| Thioureas | Isothiocyanates | Amines/anilines |
| Thioethers | Maleimides | Thiols |
| Phosphite esters | Phosphoramidites | Alcohols |
| Silyl ethers | Silyl halides | Alcohols |
| Alkyl amines | Sulfonate esters | Amines/anilines |
| Thioethers | Sulfonate esters | Thiols |
| Esters | Sulfonate esters | Carboxylic acids |
| Ethers | Sulfonate esters | Alcohols |
| Sulfonamides | Sulfonyl halides | Amines/anilines |
| Sulfonate esters | Sulfonyl halides | Phenols/alcohols |

Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. Protected derivatives are useful in the preparation of the compounds described herein or in themselves may be active as inhibitors. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Protecting or blocking groups may be selected from:

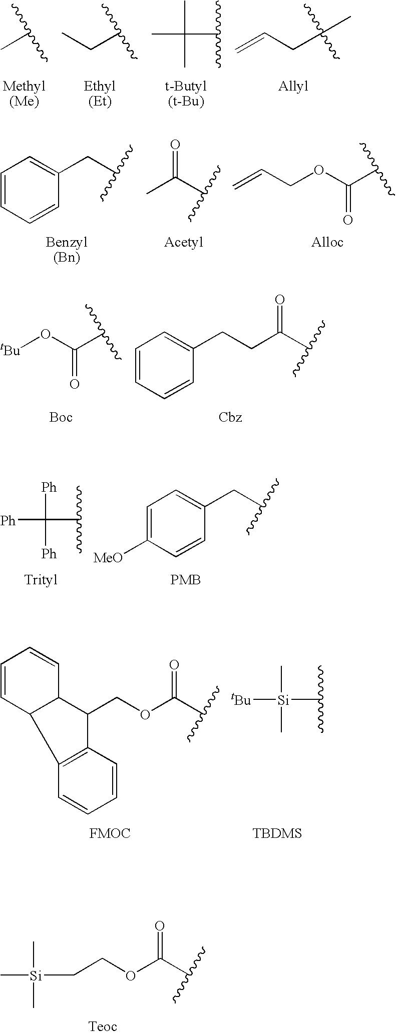

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference in their entirety.

Further Forms of the Compounds

Isomers

The compounds described herein may exist as geometric isomers. The compounds described herein may possess one or more double bonds. The compounds presented herein include all cis, tram, syn, anti, entgegen (E), and zusammen (Z) isomers as well as the corresponding mixtures thereof. In some situations, compounds may exist as tautomers. The compounds described herein include all possible tautomers within the formulas described herein.

The compounds described herein may possess one or more chiral centers and each center may exist in the R or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion may also be useful for the applications described herein.

In some embodiments, the compounds described herein can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981, herein incorporated by reference in its entirety.

Labeled Compounds

It should be understood that the compounds described herein include their isotopically-labeled equivalents, including their use for treating disorders. For example, the invention provides for methods of treating diseases, by administering isotopically-labeled compounds of Formulas I-VIII. The isotopically-labeled compounds described herein can be administered as pharmaceutical compositions. Thus, the compounds described herein also include their isotopically-labeled isomers, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹¹C, ¹³C, ¹⁴C, ¹⁵N, ¹⁸0, ¹⁷O, ³¹P, ³²P, ³⁵S, ¹⁸F, and ³⁶Cl, respectively. Compounds described herein, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds, pharmaceutically acceptable salts, esters, prodrugs, solvates, hydrates or derivatives thereof can generally be prepared by carrying out procedures described herein, by substituting a readily available isotopically labeled reagent for a non-isotopically labeled reagent.

The compounds described herein may be labeled by other means, including, but not limited to, the use of chromophores or fluorescent moieties, bioluminescent labels, or chemiluminescent labels.

Pharmaceutically Acceptable Salts

The compounds described herein may also exist as their pharmaceutically acceptable salts, which may also be useful for treating disorders. For example, the invention provides for methods of treating diseases, by administering pharmaceutically acceptable salts of the compounds described herein. The pharmaceutically acceptable salts can be administered as pharmaceutical compositions.

Thus, the compounds described herein can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts can also be prepared by reacting the free acid form of the compounds described herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Solvates

The compounds described herein may also exist in various solvated forms, which may also be useful for treating disorders. For example, the invention provides for methods of treating diseases, by administering solvates of the compounds described herein. The solvates can be administered as pharmaceutical compositions. Preferably the solvates are pharmaceutically acceptable solvates.

Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Polymorphs

The compounds described herein may also exist in various polymorphic states, all of which are herein contemplated, and which may also be useful for treating disorders. For example, the invention provides for methods of treating diseases, by administering polymorphs of the compounds described herein. The various polymorphs can be administered as pharmaceutical compositions.

Thus, the compounds described herein include all their crystalline forms, known as polymorphs. Polymorphs include the different crystal packing arrangements of the same elemental composition of a compound. Polymorphs may have different X-ray diffraction patterns, infrared spectra, melting points, density, hardness, crystal shape, optical and electrical properties, stability, solvates and solubility. Various factors such as the recrystallization solvent, rate of crystallization, and storage temperature may cause a single crystal form to dominate.

Prodrugs

The compounds described herein may also exist in prodrug form, which may also be useful for treating disorders. For example, the invention provides for methods of treating diseases, by administering prodrugs of the compounds described herein. The prodrugs can be administered as pharmaceutical compositions.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:0210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Various forms of prodrugs are well known in the art. See for example Design of Prodrugs, Bundgaard, A. Ed., Elseview, 1985 and Methods in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. The prodrugs described herein include, but are not limited to, the following groups and combinations of these groups; amine derived prodrugs:

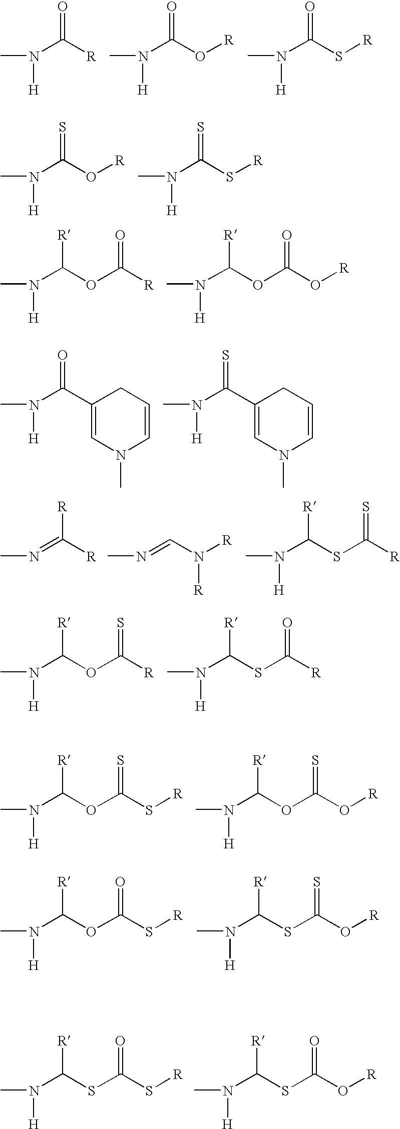

Hydroxy prodrugs include, but are not limited to acyloxyalkyl esters, alkoxycarbonyloxyalkyl esters, alkyl esters, aryl esters and disulfide containing esters.

In some embodiments, prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. The amino acid residues include but are not limited to the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. Additional types of prodrugs are also encompassed.

Prodrug derivatives of compounds described herein can be prepared by methods known to those of ordinary skill in the art (e.g., for further details see Saulnier et al., (1994), Bioorganic and Medicinal Chemistry Letters, Vol. 4, p. 1985). By way of example only, appropriate prodrugs can be prepared by reacting a non-derivatized compound of Formulas with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Compounds of Formulas I-VIII having free amino, amido, hydroxy or carboxylic groups can be converted into prodrugs. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups.

Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Phosphate ester functionalities may also be used as prodrug moieties.

Sites on the aromatic ring portions of compounds of the compounds described herein may be susceptible to various metabolic reactions, therefore incorporation of appropriate substituents on the aromatic ring structures, can reduce, minimize or eliminate this metabolic pathway.

Pharmaceutical Compositions

The present invention can be administered alone or as a pharmaceutical composition, thus the invention further provides pharmaceutical compositions and methods of making said pharmaceutical composition. In some embodiments, the pharmaceutical compositions comprise an effective amount of the compounds of Formulas I-VIII, or a pharmaceutically acceptable salt, prodrug, solvate, polymorph, tautomer or isomer thereof. The pharmaceutical composition may comprise of admixing at least one active ingredient, or a pharmaceutically acceptable salt, prodrug, solvate, polymorph, tautomer or isomer thereof, together with one or more carriers, excipients, buffers, adjuvants, stabilizers, or other materials well known to those skilled in the art and optionally other therapeutic agents. The formulations may conveniently be presented in unit dosage form and may be prepared by any methods well known in the art of pharmacy.

Examples of excipients that may be used in conjunction with the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Example of pharmaceutically acceptable carriers that may optionally be used include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments the pharmaceutical compositions are for the treatment of disorders. In some embodiments the pharmaceutical compositions are for the treatment of disorders in a mammal. In some embodiments the pharmaceutical compositions are for the treatment of cancer such as acute myeloid leukemia, thymus, brain, lung, squamous cell, skin, eye, etc.

Inhibition of Histone Deacetylase

The invention described herein provides a method of inhibiting histone deacetylase in a cell, comprising contacting a cell in which inhibition of histone deacetylase is desired with an inhibitor of histone deacetylase according to the present invention. Because compounds of the invention inhibit histone deacetylase, they are useful research tools for in vitro study of the role of histone deacetylase in biological processes. In addition, the compounds of the invention selectively inhibit certain isoforms of HDAC.

Measurement of the enzymatic activity of a histone deacetylase can be achieved using known methodologies. For example, Yoshida et al., J. Biol. Chem. 1990, 265: 17174-17179, which is incorporated by reference herein in its entirety, describes the assessment of histone deacetylase enzymatic activity by the detection of acetylated histones in trichostatin A treated cells. Taunton et al., Science 1996, 272, 408-411, which is incorporated by reference in its entirety, similarly describes methods to measure historic deacetylase enzymatic activity using endogenous and recombinant HDAC-1.

In some embodiments, the histone deacetylase inhibitor interacts with and reduces the activity of all histone deacetylases in the cell. In other embodiments according to this aspect of the invention, the histone deacetylase inhibitor interacts with and reduces the activity of fewer than all histone deacetylases in the cell. In certain other embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, and HDAC-8). In some embodiments, the histone deacetylase inhibitor of the present invention interacts with, and reduces the enzymatic activity of, a histone deacetylase that is involved in tumorigenesis. In other embodiments, the histone deacetylase inhibitors of the present invention interact with and reduce the enzymatic activity of a fungal histone deacetylase.

In some embodiments, the compounds and methods of the present invention cause an inhibition of cell proliferation of the contacted cells. The phrase “inhibiting cell proliferation” is used to denote an ability of an inhibitor of histone deacetylase to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth such as, but not limited to, a solid tumor or organ, an assessment of cell proliferation can be made by measuring the growth with calipers and comparing the size of the growth of contacted cells with non-contacted cells. In some embodiments, growth of cells contacted with the inhibitor is retarded by at least 50% as compared to growth of non-contacted cells. In other embodiments, cell proliferation is inhibited by at least 75%. In still other embodiments, cell proliferation is inhibited by 100% (i.e., the contacted cells do not increase in number). Thus, an inhibitor of histone deacetylase according to the invention that inhibits cell proliferation in a contacted cell may induce the contacted cell to undergo growth retardation, to undergo growth arrest, to undergo programmed cell death (i.e., to apoptose), or to undergo necrotic cell death.

Histone Deacetylase Mediated Disorders

Described herein are compounds, pharmaceutical compositions and methods for treating a patient suffering from a histone deacetylase mediated disorder by administering an effective amount of a compound of Formulas I-VIII, or a pharmaceutically acceptable salt, prodrug, solvate, polymorph, tautomer or isomer thereof, alone or in combination with one or more additional active ingredients.

In some embodiments, a compound of Formulas is used in the treatment of an inflammatory disease including, but not limited to, asthma, inflammatory bowel diseases such as Crohn's disease and ulcerative colitis, psoriasis, sarcoidois, and rhematoid arthritis.

In some embodiments, a compound of Formulas I-VIII is used in the treatment of an infection including, but not limited to, malaria, protozoal infections, EBV, HIV, hepatitis B and C, KSHV, toxoplasmosis and coccidiosis.

In some embodiments, a compound of Formulas I-VIII is used in the treatment of an autoimmune disorder including, but not limited to, conditions treatable by immune modulation, rheumatoid arthritis, autoimmune diabetes, lupus, multiple sclerosis, and allergies.

In some embodiments, a compound of Formulas I-VIII is used in the treatment of a neurological disorder including, but not limited to, Huntington's disease, epilepsy, neuropathic pain, depression, and bipolar disorders.

In some embodiments, a compound of Formulas I-VIII is used in the treatment of a proliferative disorder including, but not limited to, psoriasis, restenosis, autoimmune disease, proliferative responses associated with organ transplantation, and atherosclerosis.

In some embodiments, a compound of Formulas I-VIII is used in the treatment of a fibrogenic disorder including, but not limited to, scleroderma, keloid formation, pulmonary fibrosis and liver cirrhosis.

In some embodiments, a compound of Formulas I-VIII is used in the treatment of a cardiac disorder including, but not limited to, cardiovascular conditions, cardiac hypertrophy, idiopathic cardiomyopathies, and heart failure.

In some embodiments, a compound of Formulas I-VIII is used in the treatment of a hyperproliferative disorder including, but not limited to, hematologic and nonhematologic cancers, cancerous and precancerous skin lesions, leukemias, hyperplasias, fibrosis, angiogenesis, psoriasis, atherosclerosis, and smooth muscle proliferation in the blood vessels.

In some embodiments, a compound of Formulas I-VIII is used in the treatment of a metabolic disease including, but not limited to, genetic related metabolic disorders, cystic fibrosis, peroxisome biogenesis disorder, alpha-1 anti-trypsin, adrenoleukodystrophy, and spinal muscular atrophy.

In some embodiments, a compound of Formulas I-VIII is used in the treatment of a malignant disease including, but not limited to, malignant fibrous histiocytoma, malignant mesothelioma, and malignant thymoma.

In some embodiments, the compounds Formulas I-VIII are used in wound healing including, but not limited to, healing of wounds associated with radiation therapy.

In some embodiments, a compound of Formulas is used in the treatment of a stroke, ischemia, cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell lung cancer. Additional cancers to be treated with the methods and compounds of Formulas I-VIII include hematologic and non-hematologic cancers. Hematologic cancer includes multiple myeloma, leukemias, and lymphomas, acute leukemia, acute lymphocytic leukemia (ALL) and acute nonlymphocytic leukemia (ANLL), chronic lymphocytic leukemia (CLL) and chronic myelogenous leukemia (CML). Lymphoma further includes Hodgkin's lymphoma and non-Hodgkin's lymphoma, cutaneous t-cell lymphoma (CTCL) and mantle cell lymphoma (MCL). Non-hematologic cancer includes brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

In some embodiments, the cancers to treat with the methods and compositions described herein include cancers that are epithelial malignancies (having epithelial origin), and particularly any cancers (tumors) that express EGFR. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Additional types of cancers which may be treated using the compounds and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. These cancers further include cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compounds and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

Abnormal Cell Growth

Also described herein are compounds, pharmaceutical compositions and methods for inhibiting abnormal cell growth. In some embodiments, the abnormal cell growth occurs in a mammal. Methods for inhibiting abnormal cell growth comprise administering an effective amount of a compound of Formulas I-VIII, or a pharmaceutically acceptable salt, prodrug, solvate, polymorph, tautomer or isomer thereof, wherein abnormal cell growth is inhibited. Methods for inhibiting abnormal cell growth in a mammal comprise administering to the mammal an amount of a compound of Formulas I-VIII, pharmaceutically acceptable salt, prodrug, solvate, polymorph, tautomer or isomer thereof, wherein the amounts of the compound, pharmaceutically acceptable salt, prodrug, solvate, polymorph, tautomer or isomer thereof, is effective in inhibiting abnormal cell growth in the mammal.

In some embodiments, the methods comprise administering an effective amount of a compound of Formulas I-VIII, pharmaceutically acceptable salt, prodrug, solvate, polymorph, tautomer or isomer thereof, in combination with an amount of a chemotherapeutic, wherein the amounts of the compound, or pharmaceutically acceptable salt, prodrug, solvate, polymorph, tautomer or isomer thereof, and of the chemotherapeutic are together effective in inhibiting abnormal cell growth. Many chemotherapeutics are presently known in the art and can be used in combination with the compounds of the invention. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Also described are methods for inhibiting abnormal cell growth in a mammal comprising administering to the mammal an amount of a compound of Formulas I-VIII, pharmaceutically acceptable salt, prodrug, solvate, polymorph, tautomer or isomer thereof, in combination with radiation therapy, wherein the amounts of the compound, pharmaceutically acceptable salt, prodrug, solvate, polymorph, tautomer or isomer thereof, is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of the compound of Formulas I-VIII in this combination therapy can be determined as described herein.

The invention also relates to a method of and to a pharmaceutical composition of inhibiting abnormal cell growth in a mammal which comprises an amount of a compound of Formulas I-VIII, pharmaceutically acceptable salt, prodrug, solvate, polymorph, tautomer or isomer thereof, or an isotopically-labeled derivative thereof, and an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-11 (cyclooxygenase 11) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, and RS 13-0830.

Modes of Administration

Described herein are compounds of Formulas I-VIII or a pharmaceutically acceptable salt, solvate, polymorph, ester, tautomer or prodrug thereof. Also described, are pharmaceutical compositions comprising a compound of Formulas I-VIII or a pharmaceutically acceptable salt, solvate, polymorph, ester, tautomer or prodrug thereof. The compounds and compositions described herein may be administered either alone or in combination with pharmaceutically acceptable carriers, excipients or diluents, in a pharmaceutical composition, according to standard pharmaceutical practice.

Administration of the compounds and compositions described herein can be effected by any method that enables delivery of the compounds to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compound, and other suitable methods commonly known in the art. For example, compounds described herein can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the compounds and methods of the invention, e.g., as discussed in Goodman and Gilman, The Pharmacological Basis of Therapeutics (current edition).; Pergamon; and Remington's, Pharmaceutical Sciences (current edition), Mack Publishing Co., Easton, Pa.

The formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders (e.g., povidone, gelatin, hydroxypropylmethyl cellulose), inert diluents, preservative, disintegrant (e.g., sodium starch glycolate, cross-linked povidone, cross-linked sodium carboxymethyl cellulose) or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. Tablets may optionally be provided with an enteric coating, to provide release in parts of the gut other than the stomach. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

Pharmaceutical preparations may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, biocide, bacteriostats and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable lipophilic solvents or vehicles include fatty oils such as sesame oil, or synthetic fatty acid esters, such as ethyl oleate or triglycerides, or liposomes or other microparticulate systems may be used to target the compound to blood components or one or more organs. The concentration of the active ingredient in the solution may vary widely. Typically, the concentration of the active ingredient in the solution is from about 1 ng/ml to about 10 μg/ml, for example from about 10 ng/ml to about 1 μg/ml. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions

Pharmaceutical preparations may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical preparations may also be formulated in rectal compositions such as suppositories or retention enemas, e.g., containing conventional suppository bases such as cocoa butter, polyethylene glycol, or other glycerides.

Pharmaceutical preparations may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. Alternatively, a formulation may comprise a patch or a dressing such as a bandage or adhesive plaster impregnated with active ingredients and optionally one or more excipients or diluents. The amount of active ingredient present in the topical formulation may vary widely. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Formulations suitable for topical administration in the mouth include losenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

Formulations

The compounds or compositions described herein can be delivered in a vesicle, e.g., a liposome (see, for example, Langer, Science 1990, 249, 1527-1533; Treat et al., Liposomes in the Therapy of Infectious Disease and Cancer, Lopez-Bernstein and Fidler, Ed., Liss, N.Y., pp. 353-365, 1989). The compounds and pharmaceutical compositions described herein can also be delivered in a controlled release system. In one embodiment, a pump may be used (see, Sefton, 1987, CRC Crit. Ref. Biomed. Eng. 14:201; Buchwald et al. Surgery, 1980 88, 507; Saudek et al. N. Engl. J. Med. 1989, 321, (574). Additionally, a controlled release system can be placed in proximity of the therapeutic target. (See, Goodson, Medical Applications of Controlled Release, 1984, Vol. 2, pp. 115-138). The pharmaceutical compositions described herein can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium earboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of an oil-in-water emulsions. The oily phase may be a vegetable oil, for example olive oil or arachis oil, or a mineral oil, for example liquid paraffin or mixtures of these, Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solution. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump. The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutical compositions may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound or composition of the invention can be used. As used herein, topical application can include mouth washes and gargles.

Pharmaceutical compositions may be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

Doses

In one embodiment, suitable dosages are total daily dosage of between about 25 to 4000 mg/m². They can be administered in various cycles: once daily at a dose of about 200 to 600 mg; twice daily at a dose of about 200 to 400 mg; twice daily at a dose of about 200 to 400 mg intermittently (e.g. three, four, or five days per week); three times daily at a dose of about 100 to 250 mg; daily dose is 200 mg, which can be administered once-daily, twice-daily, or three-times daily; daily dose is 300 mg, which can be administered once-daily or twice-daily; daily dose is 400 mg, which can be administered once-daily or twice-daily.

In one embodiment, the compound is administered systemically to attain a blood level from about 0.01 μM to about 10 μM. In additional or further embodiments, the therapeutic composition is administered at a sufficient dosage to attain a blood level of from about 0.05 μM to about 10 μM. In additional or further embodiments, the blood level of is from about 0.1 μM to about 7 μM. In other embodiments, the compound is administered systemically to attain a blood level from about 0.01 μM to about 10 μM. In additional or further embodiments, the therapeutic composition is administered at a sufficient dosage to attain a blood level from about 0.05 μM to about 10 μM, In additional or further embodiments, the blood level is from about 0.1 μM to about 7 μM.

In one embodiment, the total dosage range is about 0.01 mg to about 5 mg per kg body weight per day. In additional or further embodiments, a total dosage will range from about 0.1 mg to about 4 mg per kg body weight per day. In additional or further embodiments, a total dosage range from about 0.1 mg to about 1 mg per kg body weight per day.

The compounds described herein can also be administered in combination with at least one second chemotherapeutic compound (e.g. pharmaceuticals, small-molecule compounds, antibodies and fragments thereof, immune system modulating proteins, antibiotics, or other biologic therapy), radiotherapy, or surgery. Such co-administration is believed to increase efficacy, provide synergistic effect, and/or provide increased therapeutic value to each agent, compound, or additional treatment (e.g. radiotherapy or surgery).

In one embodiment, the compound described herein is administered with a second chemotherapeutic compound. The co-administered compounds can be administered in a variety of cycles: the compound can be administered continuously, daily, every other day, every third day, once a week, twice a week, three times a week, bi-weekly, or monthly, while the second chemotherapeutic agent is administered continuously, daily, one day a week, two days a week, three days a week, four days a week, five days a week, six days a week, bi-weekly, or monthly. The compound and the second chemotherapeutic compound or cancer can be administered in, but are not limited to, any combination of the aforementioned cycles. In one non-limiting example, the compound is administered three times a week for the first two weeks followed by no administration for four weeks, and the second chemotherapeutic compound is administered continuously over the same six week period. In yet another non-limiting example, the compound is administered once a week for six weeks, and the second chemotherapeutic compound is administered every other day over the same six week period. In yet another non-limiting example, the compound is administered the first two days of a week, and the second chemotherapeutic compound is administered continuously for all seven days of the same week.

In addition to the administration of the compounds in cycles, the cycles themselves may consist of varying schedules. In one embodiment, a cycle is administered weekly. In additional embodiments, a cycle is administered for one week with one, two, three, four, six, or eight weeks off before repeating the cycle. In further embodiments, a cycle is administered for two weeks with one, two, three, four, six, or eight weeks off before repeating the cycle. In still further embodiments, the cycle is administered for three, four, five, or six weeks, with one, two, three, four, six, or eight weeks off before repeating the cycle.

When a compound is administered with an additional treatment such as radiotherapy, the radiotherapy can be administered at I day, 2 days, 3 days, 4 days, 5 days, 6 days, 7 days, 14 days, 21 days, or 28 days after administration of at least one cycle of a compound. In additional embodiments, the radiotherapy can be administered in any variation of timing with any variation of the aforementioned cycles for a compound. Additional schedules for co-administration of radiotherapy with cycles of a compound will be known in the art, can be further determined by appropriate testing, clinical trials, or can be determined by qualified medical professionals.

When a compound is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. Additional variations of administering compound cycles in anticipation of surgery, or after the occurrence of surgery, will be known in the art, can be further determined by appropriate testing and/or clinical trials, or can be determined by assessment of qualified medical professionals.

In addition to the aforementioned examples and embodiments of dosages, cycles, and schedules of cycles, numerous permutations of the aforementioned dosages, cycles, and schedules of cycles for the co-administration of a compound with a second chemotherapeutic compound, radiotherapy, or surgery are contemplated herein and can be administered according to the patient, type of cancer, and/or appropriate treatment schedule as determined by qualified medical professionals.

Dosage Forms

The pharmaceutical composition may, for example, be in a form suitable for oral administration as a tablet, capsule, cachet, pill, lozenge, powder or granule, sustained release formulations, solution, liquid, suspension, for parenteral injection as a sterile solution, suspension or emulsion, for topical administration as an ointment, cream, lotions, sprays, foams, gel or paste, or for rectal or vaginal administration as a suppository or pessary. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch or other cellulosic material, alginic acid and certain complex silicates and with binding agents such as sucrose, gelatin and acacia. Additionally, lubricating agents such as magnesium stearate, sodium lauryl sulfate and talc are often useful for tableting purposes. Other reagents such as an inhibitor, surfactant or solubilizer, plasticizer, stabilizer, viscosity increasing agent, or film forming agent may also be added. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Ester, Pa., 18th Edition (1990).

Combination Therapies

The compounds described herein or a pharmaceutically acceptable salt, prodrug, solvate, polymorph, tautomer or isomer thereof may be administered as a sole therapy. The compounds described herein their pharmaceutically acceptable salts, prodrug, solvates, polymorphs, tautomers or isomers may also be administered in combination with another cancer therapy or therapies. As described above, these additional cancer therapies can be, for example, surgery, radiation therapy, administration of chemotherapeutic agents and combinations of any two or all of these methods. Combination treatments may occur sequentially or concurrently and the combination therapies may be neoadjuvant therapies or adjuvant therapies.

In some embodiments, the compounds described herein can be administered with an additional therapeutic agent. In these embodiments, the compound described herein can be in a fixed combination with the additional therapeutic agent or a non-fixed combination with the additional therapeutic agent.

By way of example only, if one of the side effects experienced by a patient upon receiving one of the compounds described herein is hypertension, then it may be appropriate to administer an anti-hypertensive agent in combination with the compound. Or, by way of example only, the therapeutic effectiveness of one of the compounds described herein may be enhanced by administration of another therapeutic agent, the overall therapeutic benefit to the patient is enhanced. Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

Other therapies include, but are not limited to administration of other therapeutic agents, radiation therapy or both. In the instances where the compounds described herein are administered with other therapeutic agents, the compounds described herein need not be administered in the same pharmaceutical composition as other therapeutic agents, and may, because of different physical and chemical characteristics, be administered by a different route. For example, the compounds/compositions may be administered orally to generate and maintain good blood levels thereof, while the other therapeutic agent may be administered intravenously. The determination of the mode of administration and the advisability of administration, where possible, in the same pharmaceutical composition, is within the knowledge of the skilled clinician with the teachings described herein. The initial administration can be made according to established protocols known in the art, and then, based upon the observed effects, the dosage, modes of administration and times of administration can be modified by the skilled clinician. The particular choice of compound (and where appropriate, other therapeutic agent and/or radiation) will depend upon the diagnosis of the attending physicians and their judgment of the condition of the patient and the appropriate treatment protocol.

The compounds and compositions described herein (and where appropriate chemotherapeutic agent and/or radiation) may be administered concurrently (e.g., simultaneously, essentially simultaneously or within the same treatment protocol) or sequentially, depending upon the nature of the disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation need not be administered simultaneously or essentially simultaneously, and the initial order of administration of the compound/composition, and the chemotherapeutic agent and/or radiation, may not be important. Thus, the compounds/compositions of the invention may be administered first followed by the administration of the chemotherapeutic agent and/or radiation; or the chemotherapeutic agent and/or radiation may be administered first followed by the administration of the compounds/compositions of the invention. This alternate administration may be repeated during a single treatment protocol. With the teachings described herein, the determination of the order of administration, and the number of repetitions of administration of each therapeutic agent during a treatment protocol, would be within the knowledge of the skilled physician after evaluation of the disease being treated and the condition of the patient. For example, the chemotherapeutic agent and/or radiation may be administered first, especially if it is a cytotoxic agent, and then the treatment continued with the administration of the compounds/compositions of the invention followed, where determined advantageous, by the administration of the chemotherapeutic agent and/or radiation, and so on until the treatment protocol is complete. Thus, in accordance with experience and knowledge, the practicing physician can modify each protocol for the administration of the compound/composition for treatment according to the individual patient's needs, as the treatment proceeds. The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In some embodiments, a composition described herein is administered before the administration of one or more chemotherapeutic agents. As non-limiting examples of this embodiment of the invention, the chemotherapeutic agent can be administered hours (e.g. one, five, ten, etc.) or days (e.g., one, two, three, etc.) after administration of the composition described herein. In some embodiments, the subsequent administration is shortly after (e.g., within an hour) administration of the compound described herein.

Specific, non-limiting examples of possible combination therapies include use of the compounds of the invention with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include oral, intravenous, intraocular, subcutaneous, dermal, and inhaled topical.

In some embodiments, therapeutic agents may include chemotherapeutic agents, but are not limited to, anticancer agents, alkylating agents, cytotoxic agents, antimetabolic agents, hormonal agents, plant-derived agents, and biologic agents.

Examples of anti-tumor substances, for example those selected from, mitotic inhibitors, for example vinblastine; alkylating agents, for example cis-platin, carboplatin and cyclophosphamide; anti-metabolites, for example 5-fluorouracil, cytosine arabinside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example, interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl) propionanilide). Such conjoint treatment may be achieved by way of the simultaneous, sequential or separate dosing of the individual components of treatment.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfhydryl, carboxyl, and imidazole groups. Under physiological conditions, these drugs ionize and produce positively charged ion that attach to susceptible nucleic acids and proteins, leading to cell cycle arrest and/or cell death. Combination therapy including a HDAC inhibitor and an alkylating agent may have therapeutic synergistic effects on cancer and reduce side effects associated with these chemotherapeutic agents.

Cytotoxic agents are a group of drugs that produced in a manner similar to antibiotics as a modification of natural products. Examples of cytotoxic agents include, but are not limited to, anthracyclines (e.g. doxorubicin, daunorubicin, epirubicin, idarubicin and anthracenedione), mitomycin C, bleomycin, dactinomycin, plicatomycin. These cytotoxic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including a HDAC inhibitor and an cytotoxic agent may have therapeutic synergistic effects on cancer and reduce side effects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including a HDAC inhibitor and an antimetabolic agent may have therapeutic synergistic effects on cancer and reduce side effects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Combination therapy including a HDAC inhibitor and a hormonal agent may have therapeutic synergistic effects on cancer and reduce side effects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including a HDAC inhibitor and a plant-derived agent may have therapeutic synergistic effects on cancer and reduce side effects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including a HDAC inhibitor and a biologic agent may have therapeutic synergistic effects on cancer, enhance the patient's immune responses to tumorigenic signals, and reduce potential side effects associated with this chemotherapeutic agent.

For the treatment of oncologic diseases, proliferative disorders, and cancers, compounds according to the present invention may be administered with an agent selected from the group comprising: aromatase inhibitors, antiestrogen, anti-androgen, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, other HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, caminomycin, idarubicin, epirubicin, esorubicin, mitoxantrone, bleomycins A, A₂, and B, camptothecin, Irinotecan®, Topotecan®, 9-aminocamptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin®, Rituxan®, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva®, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with HDAC inhibitor include, but are not limited to, interleukin 2 (IL-2), and interleukin 4 (IL-4), interleukin 12 (IL-12).

Interferons include more than 23 related subtypes with overlapping activities, all of the IFN subtypes within the scope of the present invention. IFN has demonstrated activity against many solid and hematologic malignancies, the later appearing to be particularly sensitive.

Other cytokines that may be used in conjunction with a HDAC inhibitor include those cytokines that exert profound effects on hematopoiesis and immune functions. Examples of such cytokines include, but are not limited to erythropoietin, granulocyte-CSF (filgastin), and granulocyte, macrophage-CSF (sargramostim). These cytokines may be used in conjunction with a HDAC inhibitor to reduce chemotherapy-induced myelopoietic toxicity.

Other immuno-modulating agents other than cytokines may also be used in conjunction with a HDAC inhibitor to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including HDAC inhibitor and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺ pre-B and mature B cells. RITUXAN® is used as single agent for the treatment of patients with relapsed or refractory low-grade or follicular, CD20⁺, B cell non-Hodgkin's lymphoma. Combination therapy including HDAC inhibitor and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, DPC-4, NF-1, NF-2, RB, p53, WT1, BRCA1 and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. Combination therapy including a HDAC inhibitor and a tumor suppressor may have therapeutic synergistic effects on patients suffering from various forms of cancers.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAM include, but are not limited to gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

An additional component may be used in the combination to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

For the treatment of inflammatory diseases and pain, compounds according to the present invention may be administered with an agent selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

For the treatment of inflammatory diseases and pain, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. For the treatment of opthalmologic disorders and diseases of the eye, compounds according to the present invention may be administered with an agent selected from the group comprising: beta-blockers, carbonic anhydrase inhibitors, α- and β-adrenergic antagonists including a1-adrenergic antagonists, α2 agonists, miotics, prostaglandin analogs, corticosteroids, immunosuppressant agents, timolol, betaxolol, levobetaxolol, carteolol, levobunolol, propranolol, brinzolamide, dorzolamide, nipradilol, iopidine, brimonidine, pilocarpine, epinephrine, latanoprost, travoprost, bimatoprost, unoprostone, dexamethasone, prednisone, methylprednisolone, azathioprine, cyclosporine, and immunoglobulins.

For the treatment of autoimmune disorders, compounds according to the present invention may be administered with an agent selected from the group comprising: corticosteroids, immunosuppressants, prostaglandin analogs and antimetabolites, dexamethasome, prednisone, methylprednisolone, azathioprine, cyclosporine, immunoglobulins, latanoprost, travoprost, bimatoprost, unoprostone, infliximab, rutuximab and methotrexate.

For the treatment of metabolic disorders, compounds according to the present invention may be administered with an agent selected from the group comprising: insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, non-glitazone type PPARS agonist, tformin, Glipizide, glyburide, Amaryl, meglitinides, nateglinide, repaglinide, PT-112, SB-517955, SB4195052, SB-216763, N,N-57-05441, N,N-57-05445, GW-0791, AGN-.sup.194.sup.204, T-1095, BAY R3401, acarbose Exendin-4, DPP728, LAF237, vildagliptin, MK-0431, saxagliptin, GSK23A, pioglitazone, rosiglitazone, (R)-1-{4-[5-methyl-2-(4-trifluoromethyl-phenyl)-oxazol-4-ylmethoxy]-benze-nesulfonyl}2,3-dihydro-1H-indole-2-carboxylic acid described in the patent application WO 03/043985, as compound 19 of Example 4, and GI-262570.

For the combinational treatment and uses described herein, the administration of the compound/composition with a therapeutic agent, surgery, and/or radiation therapy may cause one or more undesirable side effects from the combination treatment. Such side effects may include, for example, nausea, vomiting, immunosuppression and susceptibility to infections, anemia and pain. It is, therefore, beneficial to the patient that these side effects are mitigated or abrogated. Additional therapeutic agents for treatment of these side effects may be administered along with the combination treatment.

In some embodiments, the combination treatments with the invention described herein can be administered with a therapeutic agent specific for the treatment of side effects. In these embodiments, the combination treatments with the invention described herein can be fixed with the additional therapeutic agent specific for the treatment of side effects or non-fixed with the additional therapeutic agent for treatment of side effects.

In applications with administration of the therapeutic agent for treatment of side effects with the combination treatments as described, the therapeutic agent for treatment of side effects may be administered concurrently (e.g., simultaneously, essentially simultaneously or within the same treatment protocol) or sequentially, depending upon the nature and onset of the side effect, the condition of the patient, and the actual choice of chemotherapeutic agent and/or radiation to be administered in conjunction (i.e., within a single treatment protocol) with the compound/composition. For a non-limiting example, an anti-nausea drug may be prophylactically administered prior to combination treatment with the compound and radiation therapy. For another non-limiting example, an agent for rescuing immuno-suppressive side effects is administered to the patient subsequent to the combination treatment of compound and another chemotherapeutic agent. The routes of administration for the therapeutic agent for side effects can also differ than the administration of the combination treatment. The determination of the mode of administration for treatment of side effects and the advisability of administration, where possible, in the same pharmaceutical composition, is within the knowledge of the skilled clinician with the teachings described herein. The initial administration can be made according to established protocols known in the art, and then, based upon the observed effects, the dosage, modes of administration and times of administration can be modified by the skilled clinician. The particular choice of therapeutic agent for treatment of side effects will depend upon the diagnosis of the attending physicians and their judgment of the condition of the patient and the appropriate treatment protocol.

In some embodiments, therapeutic agents specific for treating side effects may by administered before the administration of the combination treatment described. In other embodiments, therapeutic agents specific for treating side effects may by administered simultaneously with the administration of the combination treatment described. In another embodiments, therapeutic agents specific for treating side effects may by administered after the administration of the combination treatment described

In some embodiments, therapeutic agents specific for treating side effects may include, but are not limited to, anti-emetic agents, immuno-restorative agents, antibiotic agents, anemia treatment agents, and analgesic agents for treatment of pain and inflammation.

Anti-emetic agents are a group of drugs effective for treatment of nausea and emesis (vomiting). Cancer therapies frequently cause urges to vomit and/or nausea. Many anti-emetic drugs target the 5-HT₃ seratonin receptor which is involved in transmitting signals for emesis sensations. These 5-HT₃ antagonists include, but are not limited to, dolasetron (Anzemet®), granisetron (Kytril®), ondansetron (Zofran®), palonosetron and tropisetron. Other anti-emetic agents include, but are not limited to, the dopamine receptor antagonists such as chlorpromazine, domperidone, droperidol, haloperidol, metaclopramide, promethazine, and prochlorperazine; antihistamines such as cyclizine, diphenhydramine, dimenhydrinate, meclizine, promethazine, and hydroxyzine; lorazepram, scopolamine, dexamethasone, Emetrol®, propofol, and trimethobenzamide. Administration of these anti-emetic agents in addition to the above described combination treatment will manage the potential nausea and emesis side effects caused by the combination treatment.

Immuno-restorative agents are a group of drugs that counter the immuno-suppressive effects of many cancer therapies. The therapies often cause myelosuppression, a substantial decrease in the production of leukocytes (white blood cells). The decreases subject the patient to a higher risk of infections. Neutropenia is a condition where the concentration of neutrophils, the major leukocyte, is severely depressed. Immuno-restorative agents are synthetic analogs of the hormone, granulocyte colony stimulating factor (G-CSF), and act by stimulating neutrophil production in the bone marrow. These include, but are not limited to, filgrastim (Neupogen®), PEG-filgrastim (Neulasta®) and lenograstim. Administration of these immuno-restorative agents in addition to the above described combination treatment will manage the potential myelosupression effects caused by the combination treatment.

Antibiotic agents are a group of drugs that have anti-bacterial, anti-fungal, and anti-parasite properties. Antibiotics inhibit growth or causes death of the infectious microorganisms by various mechanisms such as inhibiting cell wall production, preventing DNA replication, or deterring cell proliferation. Potentially lethal infections occur from the myelosupression side effects due to cancer therapies. The infections can lead to sepsis where fever, widespread inflammation, and organ dysfunction arise. Antibiotics manage and abolish infection and sepsis and include, but are not limited to, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin. Administration of these antibiotic agents in addition to the above described combination treatment will manage the potential infection and sepsis side effects caused by the combination treatment.

Anemia treatment agents are compounds directed toward treatment of low red blood cell and platelet production. In addition to myelosuppression, many cancer therapies also cause anemias, deficiencies in concentrations and production of red blood cells and related factors. Anemia treatment agents are recombinant analogs of the glycoprotein, erythropoietin, and function to stimulate erythropoesis, the formation of red blood cells. Anemia treatment agents include, but are not limited to, recombinant erythropoietin (EPOGEN®, Dynopro®) and Darbepoetin alfa (Aranesp®). Administration of these anemia treatment agents in addition to the above described combination treatment will manage the potential anemia side effects caused by the combination treatment.

Pain and inflammation side effects arising from the described herein combination treatment may be treated with compounds selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

For the treatment of pain and inflammation side effects, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. Administration of these pain and inflammation analgesic agents in addition to the above described combination treatment will manage the potential pain and inflammation side effects caused by the combination treatment.

Kits

The compounds, compositions and methods described herein provide kits for the treatment of disorders, such as the ones described herein. These kits comprise a compound, compounds or compositions described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer. The packaging material may comprise a container for housing the composition and optionally a label affixed to the container. The kit may also optionally comprise additional components, such as syringes for administration of the composition. The kit may comprise the composition in single or multiple dose forms.

The compounds described herein can be utilized for diagnostics and as research reagents. For example, the compounds described herein, either alone or in combination with other compounds, can be used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Besides being useful for human treatment, the compounds and formulations of the present invention are also useful for veterinary treatment of companion animals, exotic animals and farm animals, including mammals, rodents, and the like. More preferred animals include horses, dogs, and cats.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### EXAMPLES

The present invention is further illustrated by the following examples, which should not be construed as limiting in any way. The experimental procedures to generate the data shown are discussed in more detail below. The invention has been described in an illustrative manner, and it is to be understood that the terminology used is intended to be in the nature of description rather than of limitation.

I. Exemplary Compounds

It should be understood that the following are provided for exemplary purposes and additional compounds and compounds with the additional substitutions are contemplated by the present invention. For example, where a substituent is indicated in the para position of a ring, it should be understood that the substituent may be in the ortho or meta position instead or that there may be an additional substituent in the ortho or meta positions. Also, where a substituent is exemplified on one compound, it should be understood that that substituent could also be attached to any of the other compounds described herein.

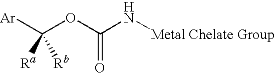

### Example 1

### General Synthesis of Compounds of the Formula 1

Compounds of the Formula 1 can be synthesized according to Scheme 1, where AR, Rₐ and R_{b} are as defined herein; and the metal chelate group is (X)g-Y-M as defined herein.

When appropriate, protecting groups are used prior to performing the reaction outlined below, and may or may not be removed upon completion of the synthesis. The individual starting materials are synthesized according to methods known in the art (or described herein) or are commercially available.

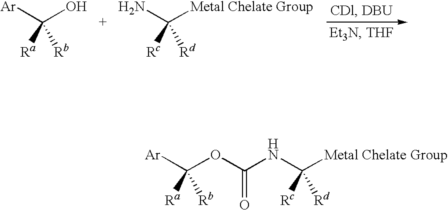

Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of the aminomethyl substituted metal chelate group (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

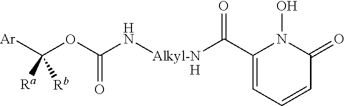

### Example 2A

### General Synthesis of Compounds of the Formula 2A

Compounds of the Formula 2A are synthesized according to Scheme 2A, where Ar is an optionally substituted C₅-C₁₅ aryl or an optionally substituted C₅-C₁₅ heteroaryl group, Rₐ and R_{b} are each independently hydrogen, halogen, —CN, -L-OH, -L-NH₂, a solubilising group, or a substituted or unsubstituted group selected from -L-alkyl, -L-alkenyl, -L-alkynyl, -L-cycloalkyl, -L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), or —S(O)₂; and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

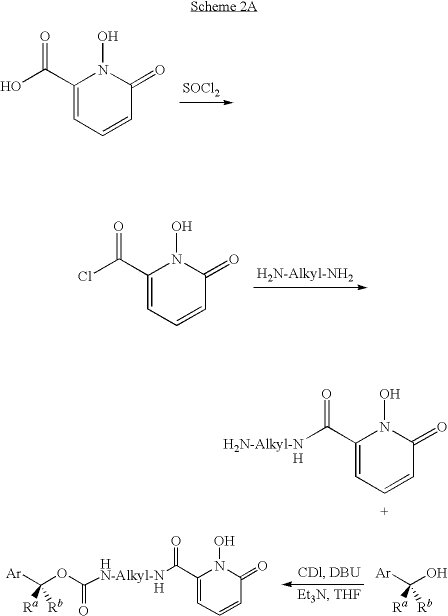

Note: Protecting groups may be used, such as protection of the N-hydroxy group during reaction with aryl diamine and/or aryl methanol, (see for example Xu et. al., J. Med. Chem., 2002, 45, 3963.)

Step 1: 1-Hydroxy-2(1H)-pyridinone-6-carboxylic acid is prepared according to published procedures (Scarrow, R. C. et al., Inorg. Chem. 1985 24, 954).

Step 2: Thionyl chloride (10 mL) is added to a suspension of 1-hydroxy-2(1H)-pyridinone-6-carboxylic acid (3.2 mmol) in dry THF (10 mL) and the mixture heated at reflux for 4 hours. Volatiles are removed by bulb-to-bulb distillation and the resulting residue suspended in dry acetone, filtered, washed with additional acetone and dried in vacuo, to give 1-hydroxy-2(1H)-pyridinone-6-carboxylic acid chloride.

Step 3: Alkyldiamine is added to a suspension of 1-hydroxy-2(1H)-pyridinone-6-carboxylic acid chloride (6.7 mmol) in dry THF (15 mL), resulting in dissolution of solids. The mixture is stirred at room temperature for 30 mins and the volatiles removed in vacuo. The resulting solid is purified by column chromatography to provide N-aminoalkyl-1-hydroxy-2(1H)-pyridinone-6-carboxamide.

Step 4: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of N-aminoalkyl-1-hydroxy-2(1H)-pyridinone-6-carboxamide (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Examples 2B-2K

Compounds 2B-2K are synthesized as described in Example 2A using the appropriate starting materials and intermediates with selective protection and deprotection when necessary.

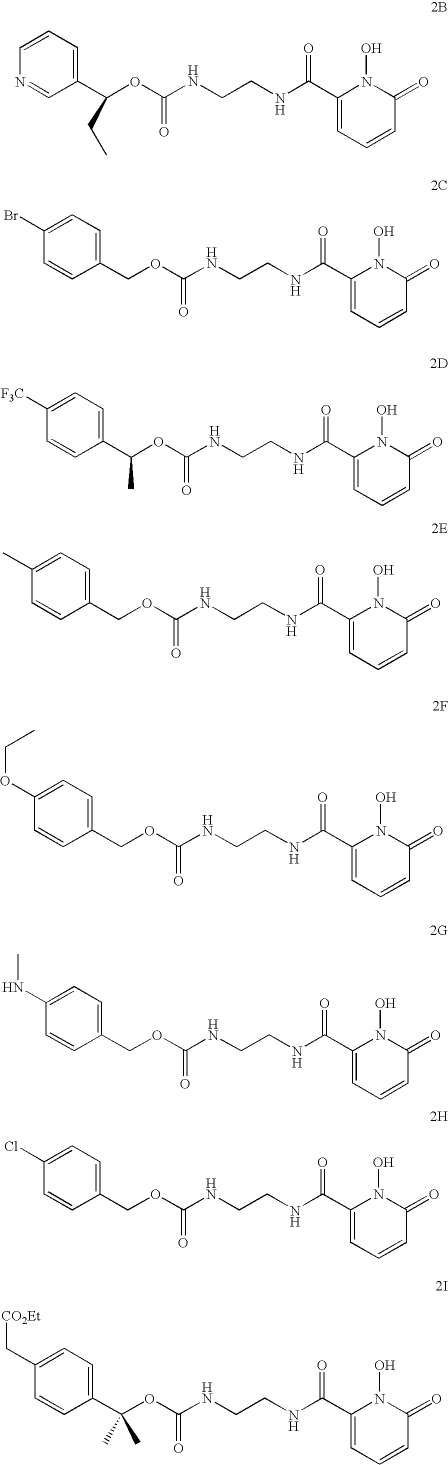

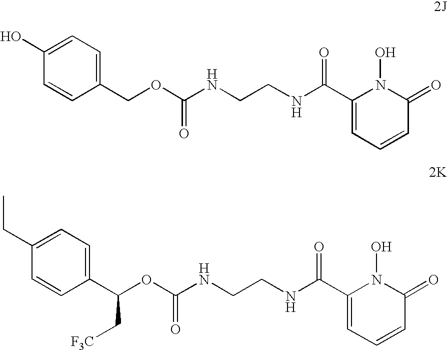

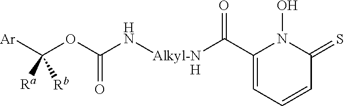

### Example 3A

### General Synthesis of Compounds of the Formula 3A

Compounds of the Formula 3A can be synthesized according to Scheme 3A, and procedures similar to those described by Abu-Dari et al., Inorg Chem, 1993, 32, 3052-3055, where Ar, Rₐ and R_{b} are as defined herein.

When appropriate, protecting groups are used prior to performing the reaction outlined below, and may or may not be removed upon completion of the synthesis. The individual starting materials are synthesized according to methods known in the art (or described herein) or are commercially available.

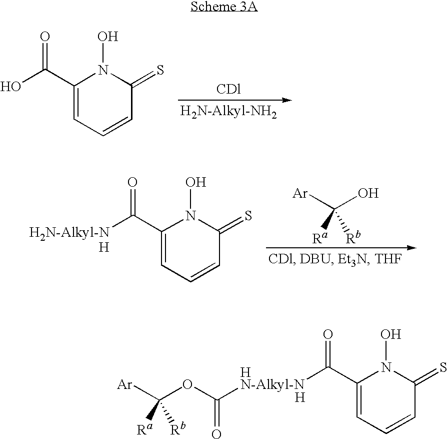

Step 1: 2-mercaptopyridine-6-carboxylic acid-1-oxide is prepared according to published procedures (see Abu-Dari et al., Inorg Chem, 1991, 30, 519-524).

Step 2: A solution of 1,1-carbonyldiimidazole (6.0 mmol) in DMF (10 mL) is added to a solution of 2-mercaptopyridine-6-carboxylic acid-1-oxide in DMF (10 mL), under nitrogen, and stirred for 15 mins. A solution of diamine (3 mmol) in DMF (10 mL) is added, and stirring continued, under nitrogen, overnight. Solvent is removed in vacuo and water (100 mL) added. Addition of HCl to pH 5 produces a precipitate which is isolated by filtration. The solid is washed with water and dissolved in aqueous sodium hydroxide (1M). The solution is filtered and re-acidified by addition of dilute HCl. The resulting precipitate is isolated by filtration, washed with water and dried under vacuum over P₂O₅.

Step 3: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of the 2-mercaptopyridine-6-aminoalkylcarboxyamide-1-oxide (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Examples 3B-3K

Compounds 313-3K are synthesized as described in Example 3A using the appropriate starting materials and intermediates with selective protection and deprotection when necessary.

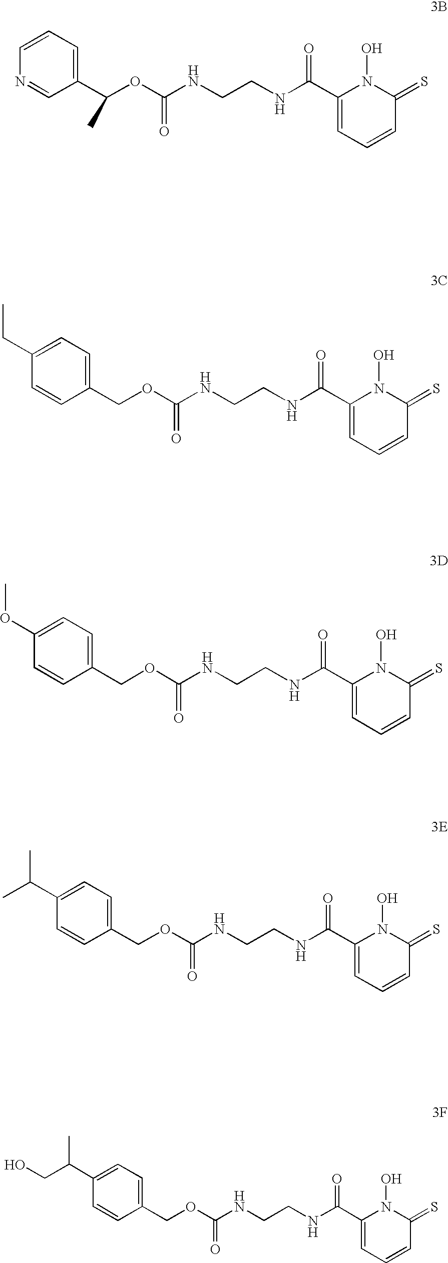

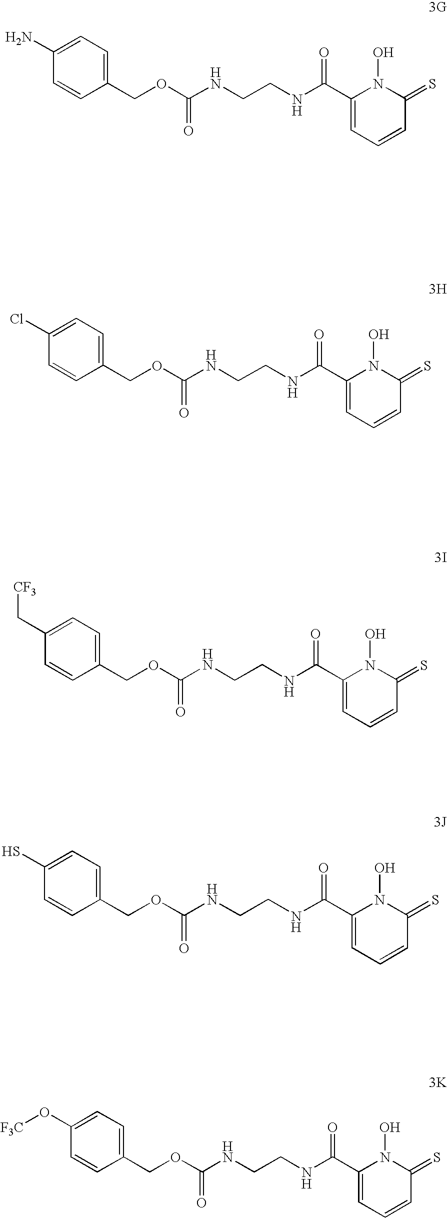

### Example 4A

### General Synthesis of Compounds of the Formula 4A

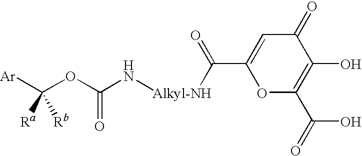

Compounds of the Formula 4A can be synthesized according to Scheme 4A, and procedures similar to those described by Pace et al., Bioorg. Med. Chem. Lett. 2004, 14, 3257, where Ar, Rₐ, and R_{b} are as defined herein.

When appropriate, protecting groups are used prior to performing the reaction outlined below, and may or may not be removed upon completion of the synthesis. The individual starting materials are synthesized according to methods known in the art (or described herein) or are commercially available.

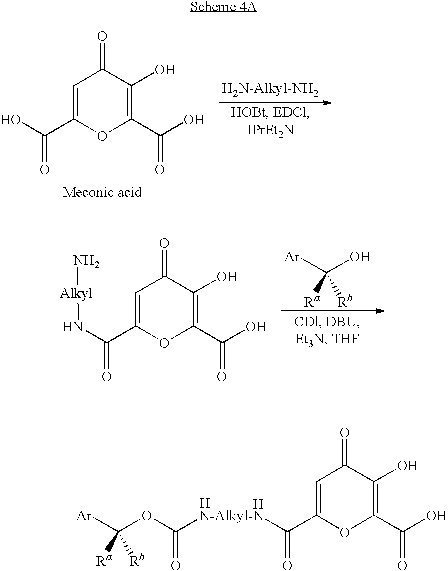

Step 1: Meconic acid, HOBt (1 equiv), EDCI (1 equiv), ⁱPr₂EtN (2 equiv) and diamine are reacted at 0° C. for 12 hours. The desired product is isolated and purified.

Step 2: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of the amine from step 1 (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Example 4B-4K

Compounds 4B-4K are synthesized as described in Example 4A using the appropriate starting materials and intermediates with selective protection and deprotection when necessary.

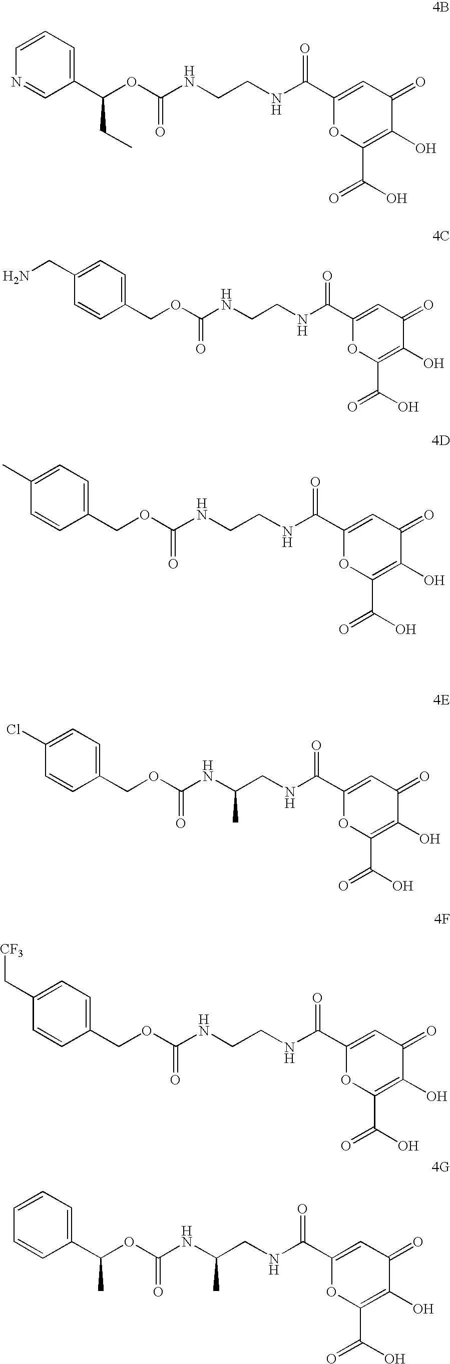

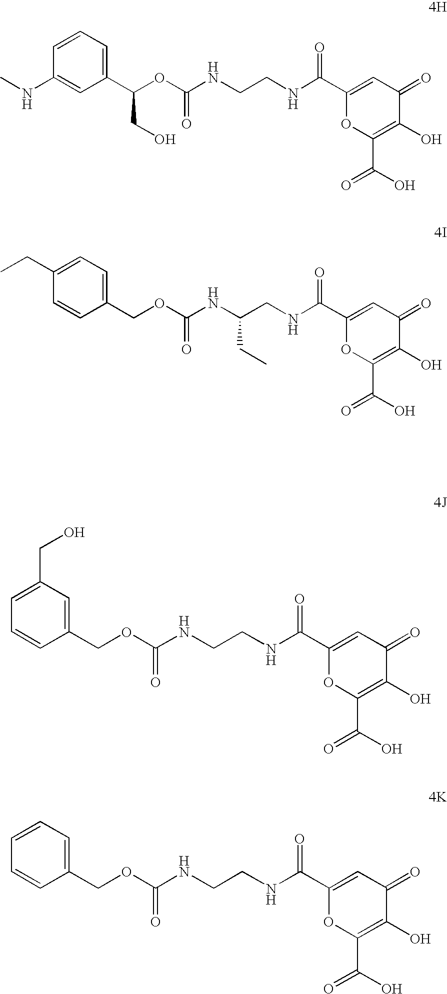

### Example 5A

### General Synthesis of Compounds of the Formula 5A

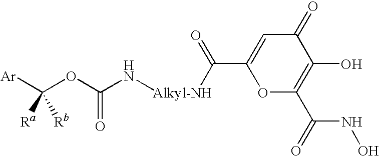

Compounds of the Formula 5A are synthesized according to Scheme 5A, where Ar, Rₐ and R_{b} are as defined herein and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

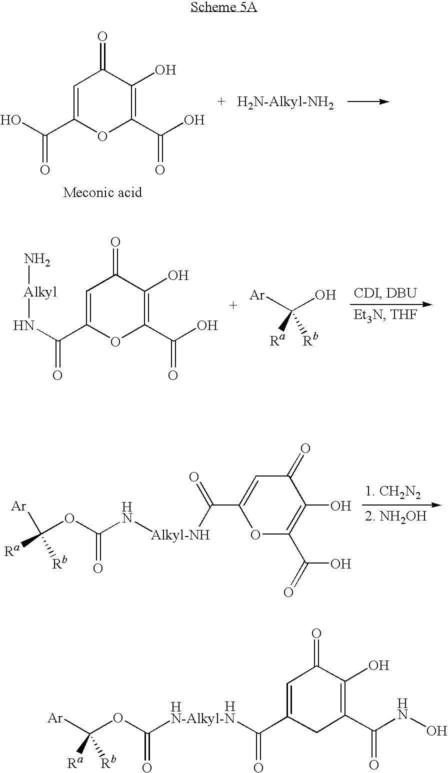

The compound prepared in example 4A is esterified with diazomethane and reacted with hydroxylamine to produce the hydroxycarboxamide derivative.

### Example 5B

### Synthesis of Compound 5B

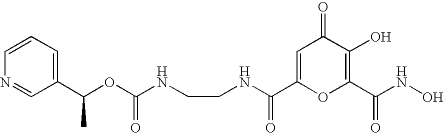

Compound 5B is synthesized according to Scheme 58.

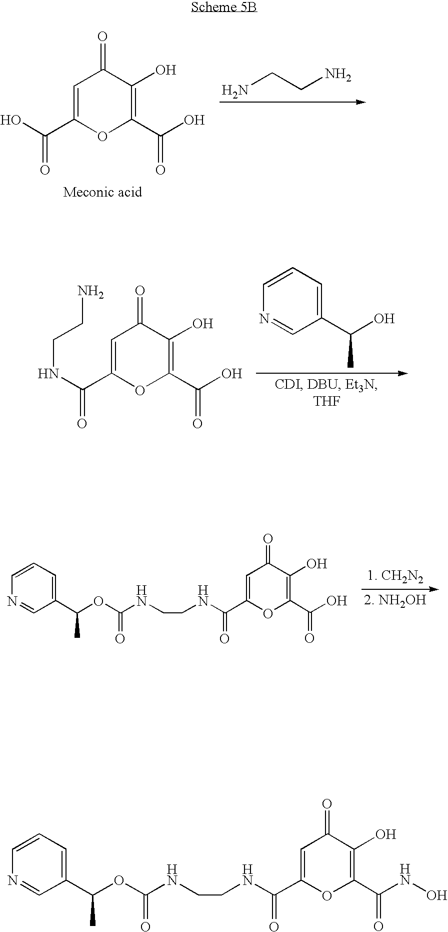

Step 1: Meconic acid, HOBt (1 equiv), EDCI (1 equiv), iPr₂EtN (2 equiv) and ethylenediamine are reacted at 0° C. for 12 hours. 6-(2-Aminoethylcarbamoyl)-3-hydroxy-4-oxo-4H-pyran-2-carboxylic acid is isolated and purified.

Step 2: (S)-1-pyridin-3-yl-ethanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of 6-(2-Aminoethylcarbamoyl)-3-hydroxy-4-oxo-4H-pyran-2-carboxylic acid (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

Step 3: The compound prepared in step 2 is esterified with diazomethane and reacted with hydroxylamine to produce the hydroxycarboxamide derivative, wherein the metal chelate group of Formula 1 is 6-(N,3-dihydroxy-4-oxo-4H-pyran-2-carboxamide).

### Example 5C-5K

Compounds 5C-5K are synthesized as described in Example 5A using the appropriate starting materials and intermediates with selective protection and deprotection when necessary.

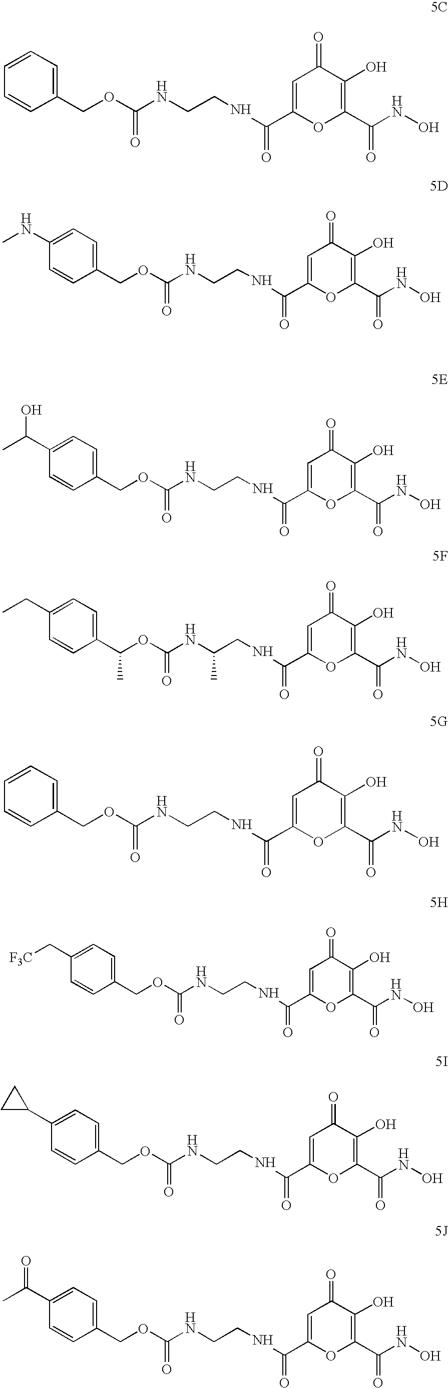

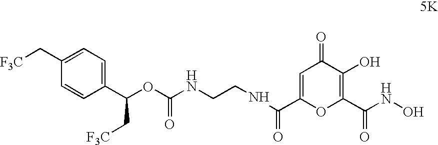

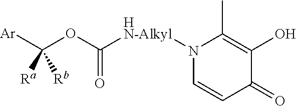

### Example 6A

### General Synthesis of Compounds of Formula 6A

Compounds of the Formula 6A are synthesized according to Scheme 6A, and procedures adapted from those described by Ellis et al., J. Med. Chem. 1996, 39, 3659 and Hare et al., J. Med. Chem. 1974, 17, 1-5 (and references therein), where Ar, Rₐ and R_{b} are as defined and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

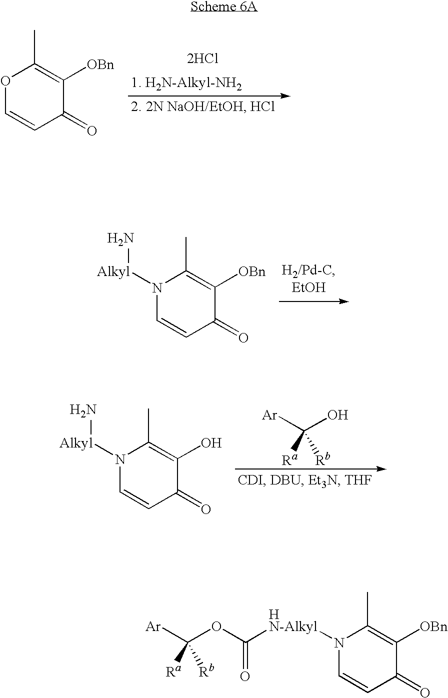

Step 1: Sodium hydroxide (2N, 190 mL, 0.38 mol) is added in portions to a solution of 3-(benzyloxy)-2-methyl-4H-pyran-4-one (53 mmol) and alkyldiamine.2HCl (58 mmol) in 39% aqueous ethanol (570 mL) with ice bath cooling. The reaction mixture is stirred at room temperature for 1 day and the volume reduced by rotary evaporation. Water (200 mL) is added and the mixture extracted several time with chloroform. The combined organic extracts are washed with saturated sodium chloride solution, dried with sodium sulfate and concentrated in vacuo. Flash chromatography and acidification with conc HCl in ethanol provides 1-(aminoalkyl)-3-(benzyloxy)-2-methylpyridin-4(1H)-one.

Step 2: Distilled solvents and glassware presoaked in 3N HCl for 15 mins is employed. Pd—C (10%, 400 mg) is added to 1-(aminoalkyl)-3-(benzyloxy)-2-methylpyridin-4(1H)-one (2.62 mmol) in 38% aqueous methanol (130 mL). The reaction mixture is stirred under H₂ at 1 atm for 5 h and then filtered through celite, which is washed with water (10 mL) and ethanol (30 mL). After removal of solvents in vacuo, chromatography on Sephadex LH-20 yields the 1-(aminoalkyl)-3-hydroxy-2-methylpyridin-4(1H)-one.

Step 3: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of the 1-(aminoalkyl)-3-hydroxy-2-methylpyridin-4(1H)-one (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Example 6B-6K

Compounds 6B-6K are synthesized as described in Example 6A using the appropriate starting materials and intermediates with selective protection and deprotection when necessary.

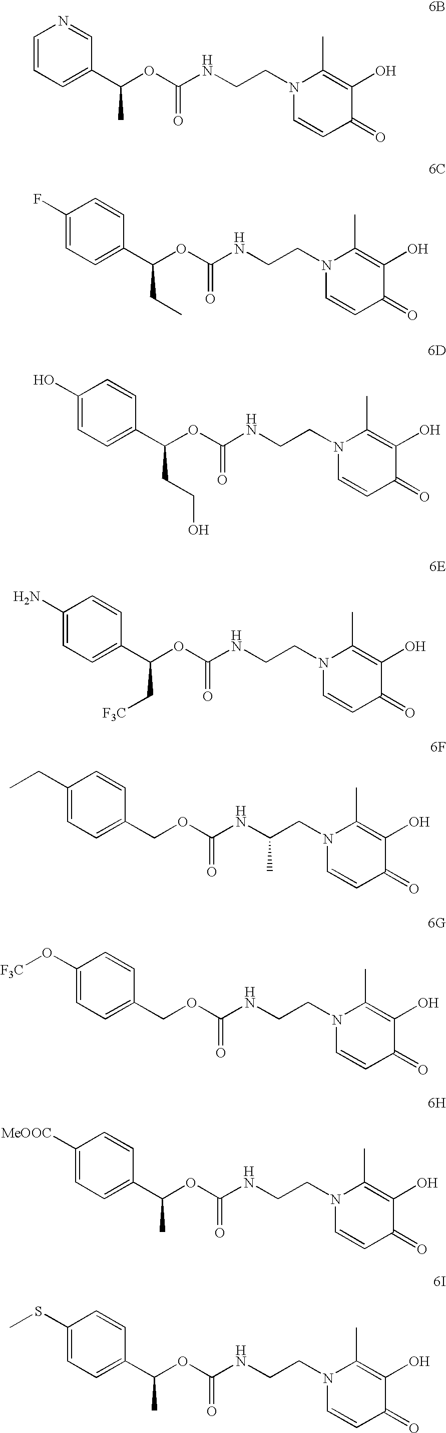

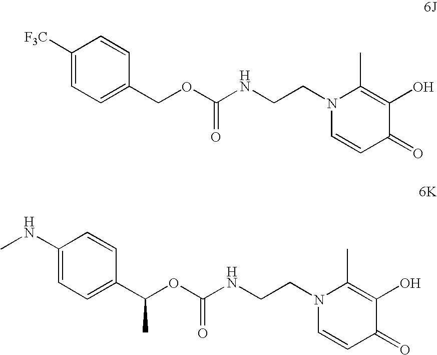

### Example 7A

### General Synthesis of Compounds of Formula 7A

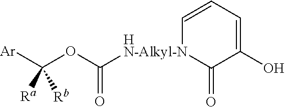

Compounds of the Formula 7A can be synthesized according to Scheme 7A, where Ar, Rₐ and R_{b} are as defined herein and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

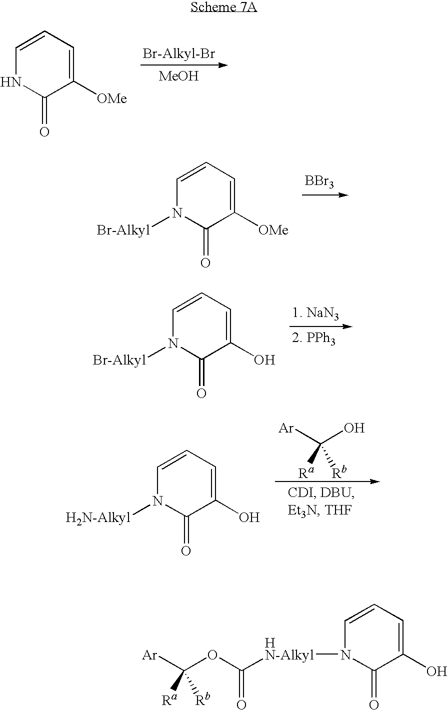

Step 1: Alkyl dihalide (0.03 mol) is added to a mixture of 3-methoxypyridin-2(1H)-one (0.015 mol) and potassium hydroxide (0.022 mol) in dry alcohol (100 mL). The solvent is removed in vacuo and the resulting residue extracted with DCM (3×5 mL). The combined organic extracts are washed with water (20 mL) and saturated sodium chloride solution (10 mL), dried over anhydrous sodium sulfate, filtered and evaporated to dryness to yield the 1-(3-haloalkyl)-3-methoxypyridin-2(1H)-one.

Step 2: A solution of borontribromide in DCM (1 equiv, 15 mL) is added to a solution of 1-(3-haloalkyl)-3-methoxypyridin-2(1H)-one (0.015 mol) in dry DCM (100 mL) under strict anhydrous conditions, at −70° C. (acetone/dry ice bath). The solution is stirred for 24 hours, cooled to −70° C. and methanol (100 mL) is slowly added. Water (2×50 mL) is added and the mixture concentrated to dryness under vacuum. The residue is adjusted to pH7 and the compound extracted into DCM (3×100 mL). The combined organic extracts are dried over anhydrous sodium sulfate, filtered, evaporated to dryness. 1-(3-haloalkyl)-3-hydroxypyridin-2(1H)-one is purified by recrystallized from petroleum ether.

Step 3: The bromo group is next displaced with azide ion to form 1-(3-azidoalkyl)-3-hydroxypyridin-2(1H)-one.

Step 4: 1-(3-azoidoalkyl)-3-hydroxypyridin-2(1H)-one is converted to 1-(3-aminoalkyl)-3-hydroxypyridin-2(1H)-one via a Staudinger reaction.

Step 5: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of 1-(3-aminoalkyl)-3-hydroxypyridin-2(1H)-one (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Example 7B-7K

Compounds 7B-7K are synthesized as described in Example 7A using the appropriate starting materials and intermediates with selective protection and deprotection when necessary.

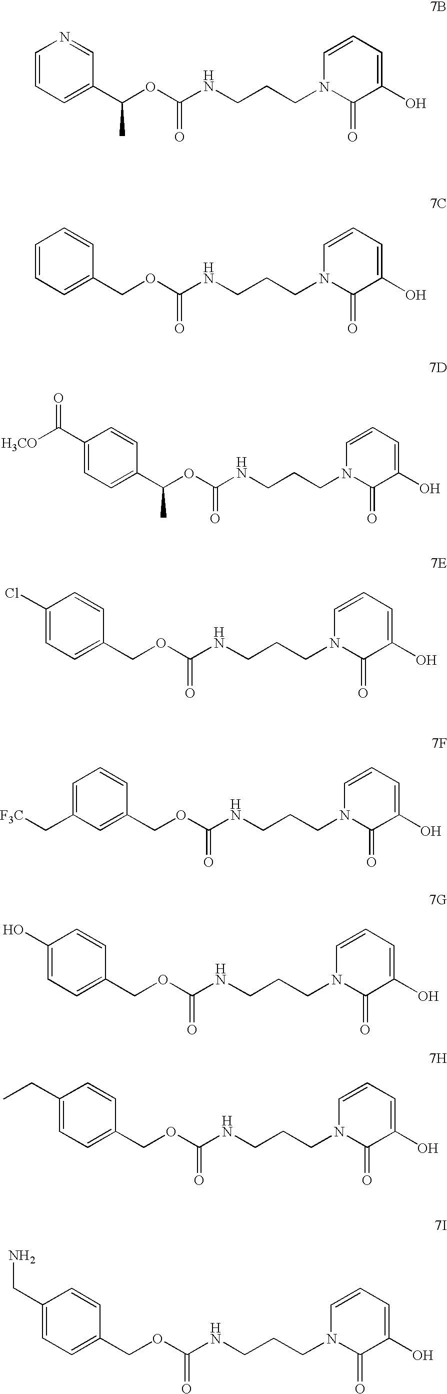

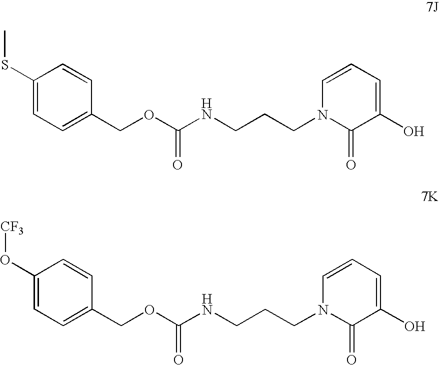

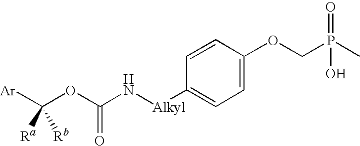

### Example 8A

### General Synthesis of Compounds of Formula 8A

Compounds of the Formula 8A can be synthesized according to Scheme 8A, and procedures described by Sheer et al., Bioorg. Med. Chem. Lett. 1997, 7, 1583 (and references therein) where Ar, Rₐ and R_{b} are as defined and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

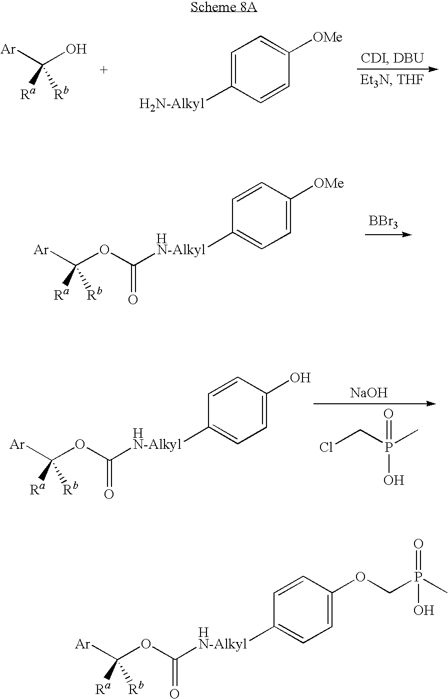

Step 1: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of the (4-methoxyphenyl)alkanamine (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the arylalkylene-4-(4-methoxyphenyl)alkylcarbamate.

Step 2: A solution of boron tribromide in DCM (1 equiv, 15 mL) is added to a solution of the arylalkylene-4-(4-methoxyphenyl)alkylcarbamate (0.015 mol) in dry DCM (100 mL) under strict anhydrous conditions, at −70° C. (acetone/dry ice bath). The solution is stirred for 24 hours, cooled to −70° C. and methanol (100 mL) is slowly added. Water (2×50 mL) is added and the mixture concentrated to dryness under vacuum. The residue is adjusted to pH7 and the compound extracted into DCM (3×100 mL). The combined organic extracts are dried over anhydrous sodium sulfate, filtered, evaporated to dryness. The arylalkylene-4-(4-hydroxyphenyl)alkylcarbamate is purified by recrystallized from petroleum ether.

Step 3: Arylalkylene-4-(4-hydroxyphenyl)alkylcarbamate and ClCH₂P(O)(Me)OH in DMF are heated to 135° C. to give the desired phosphinic acid product.

### Example 8B-8K

Compounds 8B-8K are synthesized as described in Example 8A using the appropriate starting materials and intermediates with selective protection and deprotection when necessary.

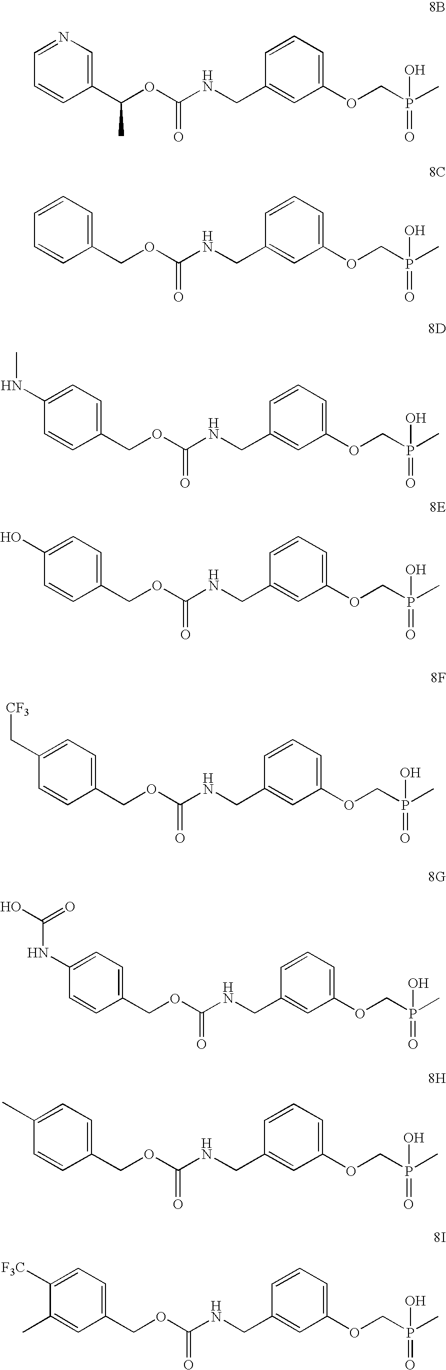

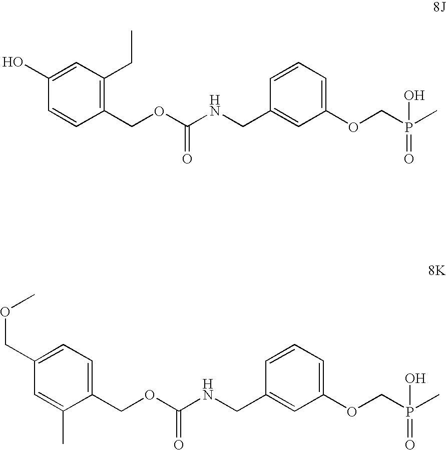

### Example 9A

### General Synthesis of Compounds of Formula 9A

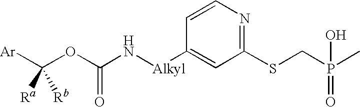

Compounds of the Formula 9A can be synthesized according to Scheme 9A, and procedures adapted from those described by Sheer et al., Bioorg. Med. Chem. Lett. 1997, 7, 1583 (and references therein) where Ar, Rₐ and R_{b} are as defined herein and alkyl is C₁-C₇ alkyl.

When appropriate, protecting groups are used prior to performing the reaction outlined below, and may or may not be removed upon completion of the synthesis. The individual starting materials are synthesized according to methods known in the art (or described herein) or are commercially available.

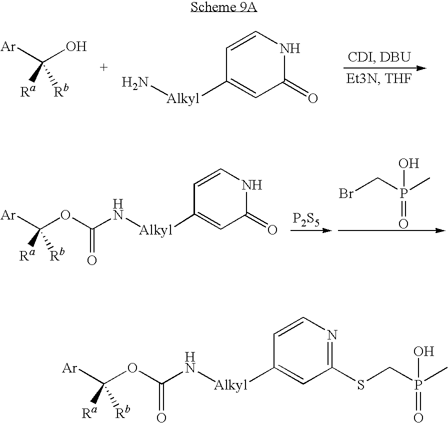

Step 1: Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of 4-(2-aminoalkyl)pyridin-2(1H)-one (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the arylmethyl 2-(1,2-dihydro-2-oxopyridin-4-yl)alkylcarbamate.

Step 2: Arylmethyl 2-(1,2-dihydro-2-oxopyridin-4-yl)alkylcarbamate is converted to the thioamide by reaction with P₂S₅.

Step 3: Arylmethyl 2-(1,2-dihydro-2-thio-pyridin-4-yl)alkylcarbamate and ClCH₂P(O)(Me)OH are heated to 135° C. in DMF to give the desired phosphinic acid product.

### Example 9B-9K

Compounds 9B-9K are synthesized as described in Example 9A using the appropriate starting materials and intermediates with selective protection and deprotection when necessary.

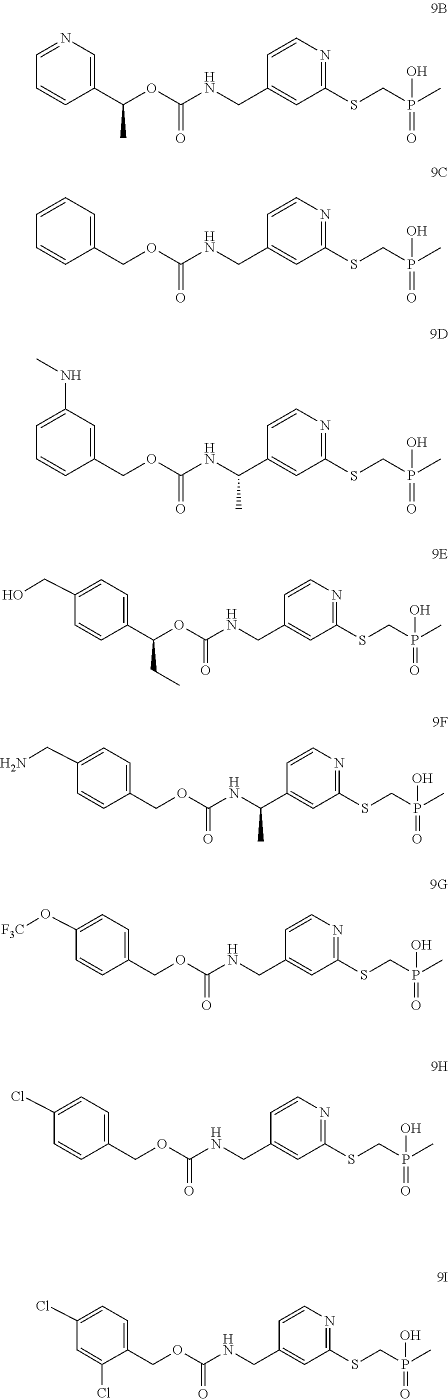

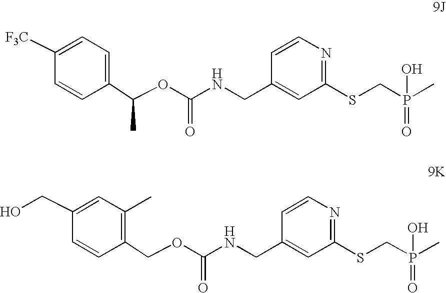

### Example 10A

### General Synthesis of Compounds of the Formula 10A

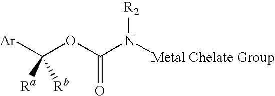

Compounds of the Formula 10A can be synthesized according to Scheme 10A, where Ar, Rₐ and R_{b} are as defined herein and the metal chelate group is (X)g-Y-M as defined herein.

When appropriate, protecting groups are used prior to performing the reaction outlined below. The individual starting materials are synthesized according to methods known in the art (or described herein) or are commercially available.

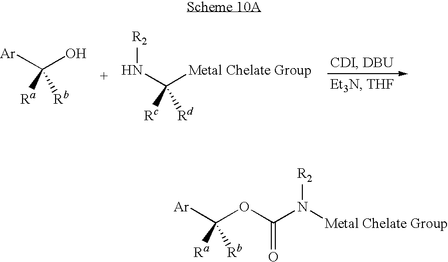

Aryl methanol (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension of NH(R²) metal chelate group (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Example 10B-10U

Compounds 10B-10Q are synthesized as described in Example 10A using the appropriate starting materials and intermediates with selective protection and deprotection when necessary.

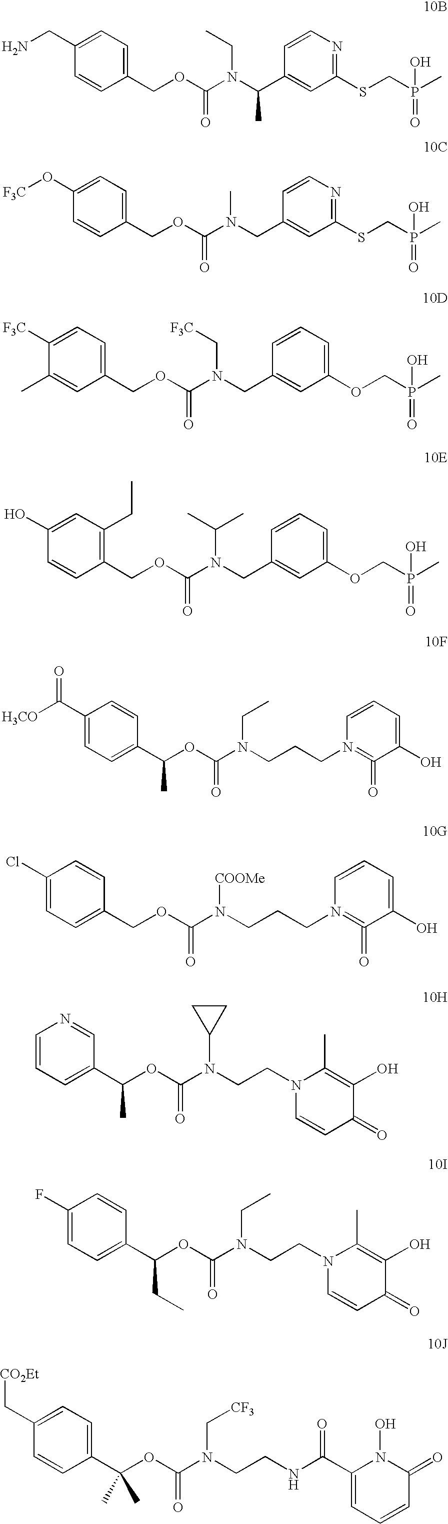

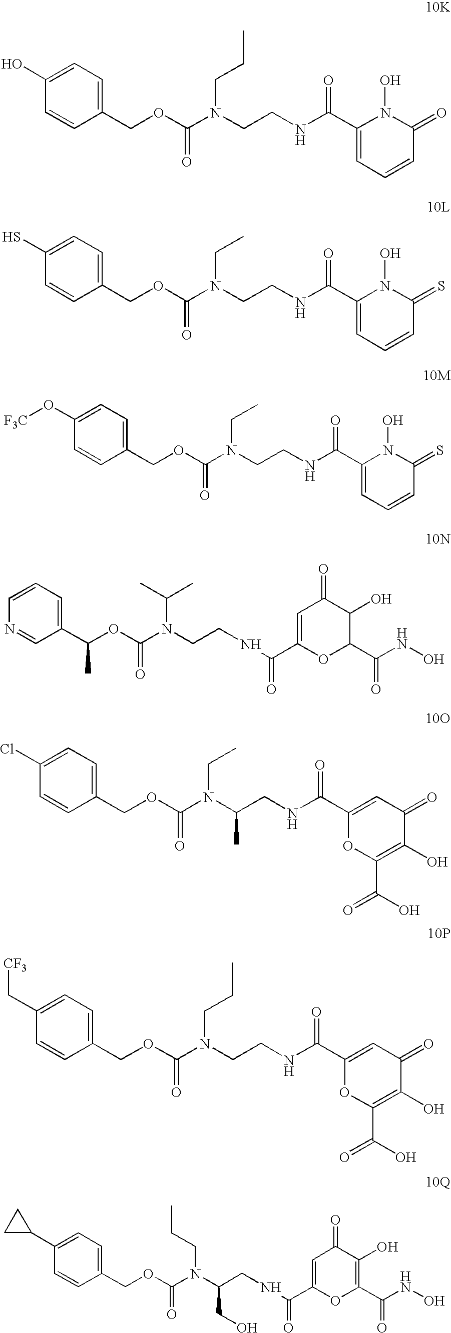

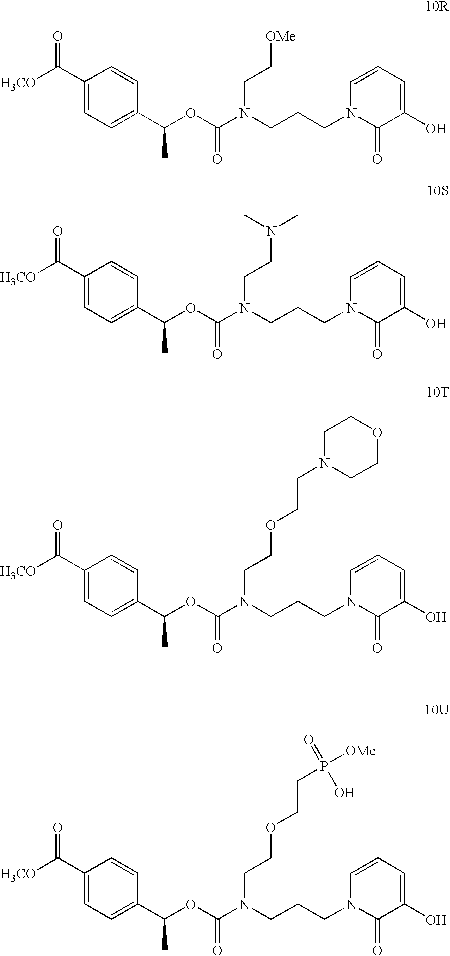

### Example 11A

### General Synthesis of Compounds of the Formula 11A

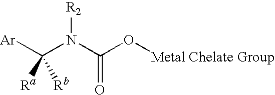

Compounds of the Formula 11A can be synthesized according to Scheme 11A, where Ar, Rₐ, R_{b} R₂ are as defined and the metal chelate group is (X)g-Y-M as defined herein.

When appropriate, protecting groups are used prior to performing the reaction outlined below. The individual starting materials are synthesized according to methods known in the art (or described herein) or are commercially available.

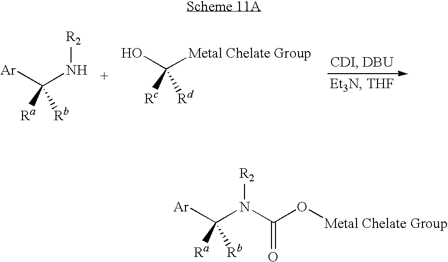

A metal chelate group comprising a methanol moiety (158 mmol) in THF (50 mL) is added to a suspension of 1,1′-carbonyldiimide (158 mmol) in THF (120 mL), at 10° C., and the mixture stirred for 1 hour at room temperature. The resulting solution is added to a suspension NH(R²)aryl (158 mmol), DBU (158 mmol) and triethylamine (158 mmol) in THF (250 mL). After stirring at room temperature for 5 hours, the solvent is removed in vacuo, and the residue dissolved in water (300 mL). The solution is acidified with HCl (pH 5) to precipitate a solid which is collected by filtration, washed with water (300 mL) and methanol (50 mL), respectively, and dried to give the desired product.

### Examples 12A-12H

Compounds 12A-12H are synthesized as described in the examples above using the appropriate starting materials and protecting groups as necessary

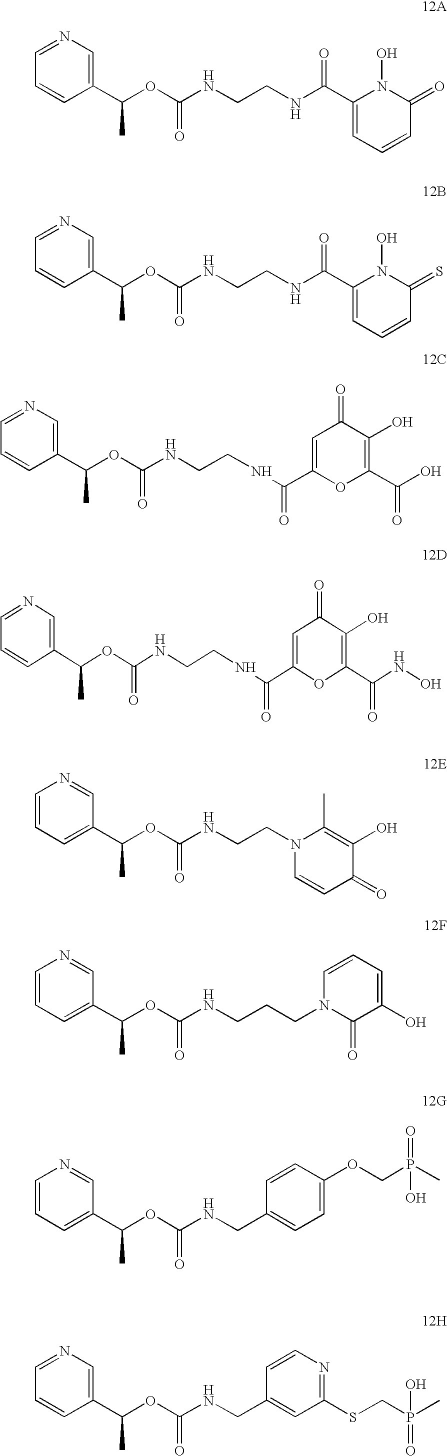

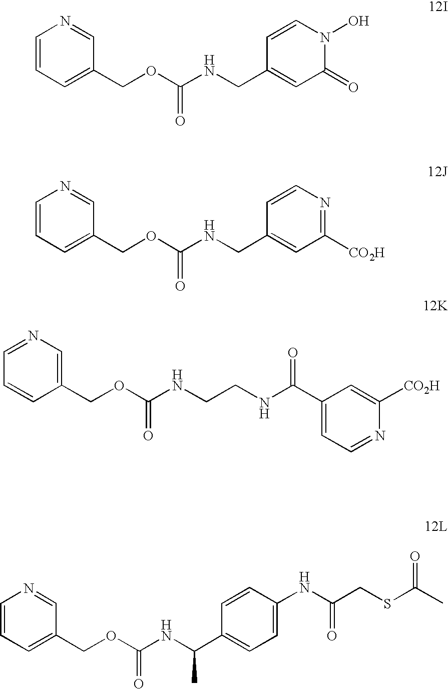

### Examples 13A-13R

Compounds 13A-13R are synthesized as described in the examples above using the appropriate starting materials and protecting groups, as required.

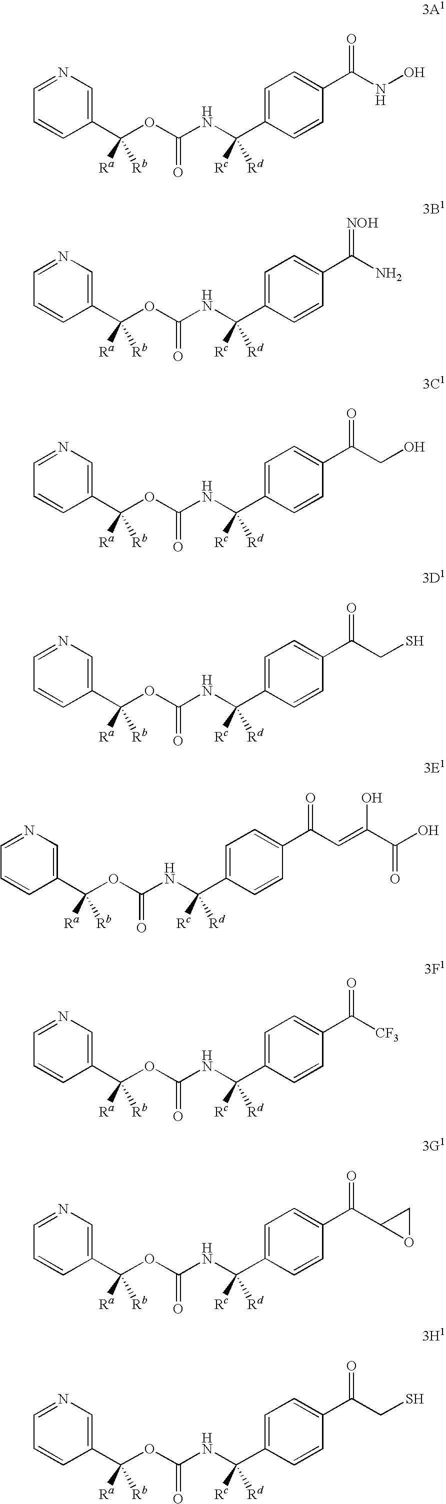

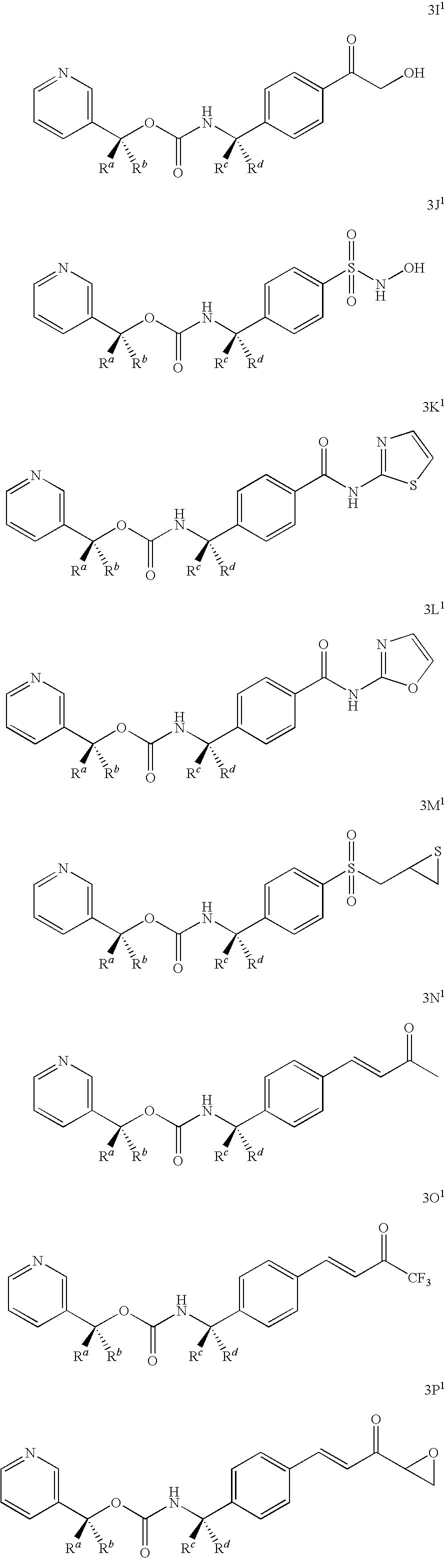

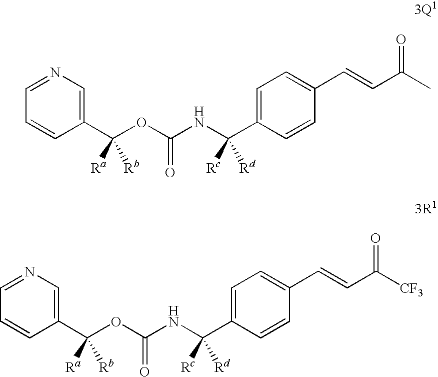

### Example 14

### Synthesis of Pyridin-3-ylmethyl (1-hydroxy-2-oxo-1,2-dihydropyridin-4-yl)methylcarbamate

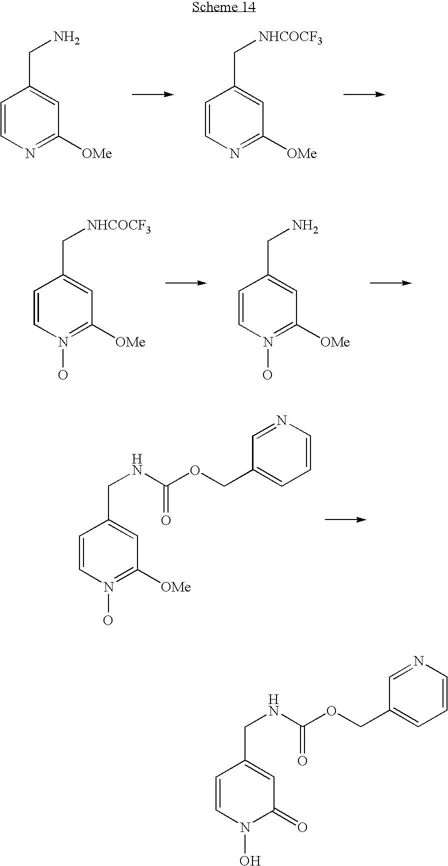

### Step 1: Synthesis of 2,2,2-trifluoro-N((2-methoxypyridin-4-yl)methyl)acetamide

A solution of 4-aminomethyl-2-methoxypyridine and trifluoroacetic anhydride was stirred at rt overnight. After this time, the product, mp 51-54° C., was isolated by standard methods in 97% yield. ¹H NMR (500 MHz, CDCl₃) δ 8.16 (d), 6.78 (d), 6.63 (s), 4.50 (d), 3.94 (s). IR (cm⁻¹): 3019, 1720, 1215, 777.

### Step 2: Synthesis of 2,2,2-trifluoro-N-((2-methoxypyridin-4-yl)methyl)acetamide N-oxide

The product of step 1 was reacted with MCPBA at rt for 30 hours to give the desired product as white needles, mp 176-178° C., in 70% yield. ¹H NMR (500 MHz, DMSO-d₆) δ 10.03 (s), 8.19 (d), 7.18 (s), 6.89 (m), 4.38 (s), 3.96 (s). IR (cm⁻¹): 3018, 1400, 1224, 788.

### Step 3: Synthesis of (2-methoxypyridin-4-yl)methanamine N-oxide

The product of step 2 was dissolved in methanol and treated with potassium carbonate at rt for 12 h. After this time the product was isolated as a yellow oil in 20% yield. ¹H NMR (500 MHz, CDCl₃) δ 8.22 (d), 6.99 (s), 6.88 (d), 4.10 (s), 3.93 (s). IR (cm⁻¹): 3366, 1692, 1494, 1204.

### Step 4: Synthesis of Pyridin-3-ylmethyl (2-methoxypyridin-4-yl)methylcarbamate N-oxide

The product of step 3 was treated with 3-hydroxymethylpyridine and carbonyl diimidazole in THF at 10° C. for 1 h and DBU, triethylamine and DMF at rt for 5 h. The desired product was isolated in 43% yield as a reddish oil. ¹H NMR (500 MHz, DMSO-d₆) δ 8.59 (s), 8.53 (d), 8.16 (d), 7.96 (d), 7.79 (d), 7.40 (d), 7.07 (s), 6.88 (d), 5.10 (d), 4.19 (d), 3.93 (s). IR (cm⁻¹): 3245, 1715, 1493, 1207, 811.

### Step 5: Synthesis of Pyridin-3-ylmethyl (1-hydroxy-2-oxo-1,2-dihydropyridin-4-yl)methylcarbamate

The product of step 4 was dissolved in dichloromethane and acetyl chloride was added and the resulting mixture heated to reflux for 2 h. At this time, acetone and water was added. After 8 h, the product was isolated in 37% yield and had a melting point of 145° C. ¹H NMR (500 MHz, DMSO-d₆) δ 8.6 (m), 8.5 (m), 7.9-7.7 (m), 7.4 (m), 6.3 (m), 6.1 (m), 5.1 (s), 4.02 (s). IR (cm⁻¹): 3242, 1721, 1666, 1252, 754. MS (M+Na): 298.

### Example 15

### Synthesis of 4-(2-((pyridin-3-ylmethoxy)carbonylamino)ethylcarbamoyl)picolinic acid

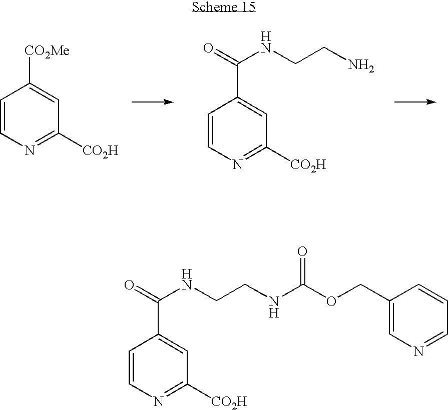

### Step 1: Synthesis of 4-(2-aminoethylcarbamoyl)picolinic acid

4-(Methoxycarbonyl)picolinic acid was treated with ethylene diamine in ethanol for 16 h at rt. The product was isolated in 43% yield as a white solid. ¹H NMR (500 MHz, D₂O) δ 7.83 (d, J=5 Hz, 1H), 7.41 (s, 1H), 7.03 (d, J=5 Hz, 1H), 2.89 (m, 2H), 2.42 (m, 2H). IR (cm⁻¹): 3019, 1720, 1215, 777.

### Step 2: Synthesis of 4-(2-((pyridin-3-ylmethoxy)carbonylamino)ethykarbamoyl)picolinic acid

The product of step 2 was treated with 3-hydroxymethylpyridine and carbonyl diimidazole in THF at 10° C. for 1 h and DBU, triethylamine and DMF at rt for 5 h. The desired product was isolated in 50% yield as a white solid. ¹H NMR (500 MHz, DMSO-d₆) δ 8.9 (bs), 8.7-8.4 (m), 7.9 (bs), 7.8 (bs), 7.5-7.3 (m), 5.1 (s), 3.4 (m), 3.3 (m). MS (M+Na): 367.

II Biological Screening

### Example 16

### Deacetylase Activity

The assay described in this example relies on the release of radioactive acetate from a radioactively labeled histone fragment by the action of HDAC enzyme. Compounds that inhibit HDAC will reduce the yield of radioactive acetate. Signal (e.g., scintillation counts) measured in the presence and absence of a test compound provides an indication of the compounds HDAC inhibitory activity. Decreased activity indicates increased inhibition by the compound.

The histone fragment can be an N-terminal sequence from histone H4, labeled with radioactively labeled acetyl groups using tritiated acetylcoenzyme A (coA) in conjunction with an enzyme which is the histone acetyltransferase domain of the transcriptional coactivator p300. 0.33 mg of peptide H4 (the N-terminal 20 amino acids of histone H4, synthesized using conventional methods), incubated with His6-tagged p300 histones acetyltransferase domain (amino acids 1195 1673, expressed in E. coli strain BLR(DE3)pLysS (Novagen, Cat. No. 69451-3) and 3H-acetyl coA (10 μL of 3.95 Ci/mmol; from Amersham) in a total volume of 300 μL of HAT buffer (50 mM TrisCl pH 8, 5% glycerol, 50 mM KCl, 0.1 mM ethylenediaminetetraacetic acid (EDTA), 1 mM dithiothreitol (DTT) and 1 mM 4-(2-aminoethyl)-benzenesulfonylfluoride (AEBSF)). The mixture is incubated at 30° C. for 45 min. The His-p300 is then removed using nickel-trinitriloacetic acid agarose (Qiagen, Cat No. 30210) and the acetylated peptide is separated from free acetyl coA by size exclusion chromatography on Sephadex G-15 (Sigma G-15-120), using distilled H₂O as the mobile phase. The radiolabeled histone fragment is then purified and incubated with a source of HDAC (e.g., an extract of HeLa cells, a rich source of HDAC, recombinantly produced HDAC1 or HDAC2). Any released acetate is then extracted into an organic phase and quantitatively determined using scintillation counting. By including a compound described herein with the source of HDAC, the compound's ability to inhibit the HDAC is determined.

### HeLa Cell Extract

A HeLa cell extract is made from HeLa cells (ATCC Ref. No. CCL-2) by freezing-thawing the cells three times in 60 mM Tris CI pH 8.0, 450 mM NaCl, 30% glycerol. Two cell volumes of extraction buffer is used and particulate material is centrifuged out (20800 g, 4° C., 10 min). The supernatant extract having deacetylase activity is then alliquoted out. This material can be frozen for storage.

### Recombinantly Produced HDAC1 and HDAC2

Full length human HDAC1 are cloned by PCR using a λgt11 Jurkat cDNA library (Clontech-HL5012b). The amplified fragment is inserted into the EcoRI-SalI sites of pFlag-CTC vector (Sigma-E5394), in frame with the Flag tag. A second PCR step is then carried out in order to amplify a fragment containing the HDAC1 sequence fused to the Flag tag. The resulting fragment is subcloned into the EcoRI-Sac1 sites of the baculovirus transfer vector pAcHTL-C (Pharmingen-21466P).

Full length human HDAC2 is subcloned into pAcHLT-A baculovirus transfer vector (Pharmingen-21464P) by PCR amplification of the EcoRI-Sac1 fragment from a HDAC2-pFlag-CTC construct. Recombinant protein expression and purification are performed by constructing HDAC1 and HDAC2 recombinant baculoviruses using BaculoGold Transfection Kit (Pharmingen-554740). The transfer vectors are co-transfected into SF9 insect cells (Pharmingen-21300C) and the recombinant viruses are amplified according to the Pharmingen Instruction Manual. The SF9 cells are maintained in serum-free SF900 medium (Gibco 10902-096).

For protein production, 2×10⁷ cells are infected with the appropriate recombinant virus for 3 days. The cells are then harvested and spun at 3,000 rpm for 5 minutes. The cells are then wash twice in PBS and resuspended in 2 pellet volumes of lysis buffer (25 mM HEPES pH 7.9, 0.1 mM EDTA, 400 mM KCl, 10% glycerol, 0.1% NP-40, 1 mM AEBSF). The resuspended cells are frozen on dry ice and thawed at 37° C. three times and then centrifuged for 10 minutes at 14,000 rpm. The supernatant is then collected and incubated with 300 μl of 50% Ni-NTA agarose bead slurry (Qiagen-30210). Incubation is carried out at 4° C. for 1 hour on a rotating wheel. The slurry is centrifuged at 500 g for 5 minutes. The beads are washed twice in 1 ml of wash buffer (25 mM HEPES pH7.9, 0.1 mM EDTA, 150 mM KCl, 10% glycerol, 0.1% NP-40, 1 mM AEBSF) and the protein is eluted 3 times in 300 μl elution buffer (25 mM HEPES pH 7.9, 0.1 mM EDTA, 250 mM KCl, 10% glycerol, 0.1% NP-40, 1 mM AEBSF) containing increasing concentrations of imidazole: 0.2 M, 0.5 M and 1 M. Each elution is performed for 5 minutes at room temperature. The eluted protein can be stored in 50% glycerol at −70° C.

### Assay Method

A source of HDAC is incubated (e.g., 2 μL of crude HeLa extract, 5 μL of HDAC1 or HDAC2; in elution buffer, as above) with 3 μL of radioactively labeled peptide along with appropriate dilutions of one or more compounds described herein (1.5 μL) in a total volume of 150 μL of buffer (20 mM Tris pH 7.4, 10% glycerol). The reaction is carried out at 37° C. for one hour and then stopped by adding 20 μL of 1 M HCl/0.4 M sodium acetate. 750 μL of ethyl acetate is then added and the samples are vortexed, centrifuged (14000 rpm, 5 min), and transferred 600 μL from the upper phase to a vial containing 3 mL of scintillation liquid (UltimaGold, Packard, Cat. No. 6013329). Measure radioactivity using a Tri-Carb 2100TR Liquid Scintillation Analyzer (Packard).

Percent activity (% activity) for each compound tested is calculated as: % activity={(S^{C}−B)/(S°−B)}×100 wherein S^{C} denotes signal measured in the presence of enzyme and the compound being tested, S° denotes signal measured in the presence of enzyme but in the absence of the compound being tested, and B denotes the background signal measured in the absence of both enzyme and compound being tested. The IC₅₀ corresponds to the concentration which achieves 50% activity.

Measurement of cell viability in the presence of increasing concentration of test compound at different time points can be used to assess both cytotoxicity and the effect of the compound on cell proliferation.

### Example 17

### Secondary Assay

### Cell Proliferation

Compounds with HDAC inhibition activity can be further evaluated using secondary cell-based assays. In this assay, the following cell lines can be used:

HeLa—Human cervical adenocarcinoma cell line (ATCC ref. No. CCL-2).

K11-HPV E7 transformed human keratinocyte line provided by Pidder Jansen-Duerr, Institut fur Biomedizinische Alternsforschung, Innsbruck, Austria.

NHEK-Ad—Primary human adult keratinocyte line (Cambrex Corp., East Rutherford, N.J., USA).

JURKAT—Human T-cell line (ATCC no. TIB-152).

### Assay Method

Cells are cultured, exposed to a compound described herein, and incubated. After incubation, the number of viable cells is then assessed using the Cell Proliferation Reagent WST-1 from Boehringer Mannheim (Cat. No. 1 644 807), described below.

The cells are placed in 96-well plates at 3-10×10³ cells/well in 100 μL of culture medium. The following day, different concentrations of one or more of the compounds described herein are added and the cells are incubated at 37° C. for 48 h. Subsequently, 10 μL/well of WST-1 reagent is added and cells are re-incubated for 1 hour. After the incubation, the absorption is measured. WST-1 is a tetrazolium salt which is cleaved to formazan dye by cellular enzymes. An expansion in the number of viable cells results in an increase in the overall activity of mitochondrial dehydrogenases in the sample. This augmentation in the enzyme activity leads to an increase in the amount of formazan dye formed, which directly correlates to the number of metabolically active cells in the culture. The formazan dye produced is quantified by a scanning multiwell spectrophotometer by measuring the absorbance of the dye solution at 450 nm wavelength (reference wavelength 690 nm).

Percent activity (% activity) in reducing the number of viable cells can be calculated for each compound tested as: % activity={(S^{C}−B)/(S°−B)}×100 wherein S^{C} denotes signal measured in the presence of the compound being tested, S° denotes signal measured in the absence of the compound tested, and B denotes the background signal measured in blank wells containing medium only. The IC₅₀ corresponds to the concentration which achieves 50% activity. IC₅₀ values are calculated using the software package Prism 3.0 (GraphPad Software Inc., San Diego, Calif.), setting top value at 100 and bottom value at 0.

Measurement of cell viability in the presence of increasing concentration of the compound tested at different time points can be used to assess both cytotoxicity and the effect of the compound on cell proliferation.

### Example 18

### Generation of GI₅₀, TGI and LC₅₀

Compounds described herein are screened for anti-cancer activity in three cell lines (5000 HCT 116 cells/wells, 5000 NCIH 460 cells/well and 5000 U251 cells/well) for their GI₅₀, TGI and LC₅₀ values (using five concentrations for each compound tested). The cell lines in DMEM containing 10% fetal bovine serum are maintained. 96 microtiter plate wells with 100 μL of cells are inoculated and maintained for 24 h at 37° C., 5% CO₂, 95% air and 100% relative humidity. The cells are inoculated and then the plate is separated with these cell lines to determine cell viability before the addition of the compounds (T₀).

Following 24-hour incubation, the compound(s) are added to the 96 well plates. Each plate contains one of the above cell lines and the following in triplicate: five different concentrations (0.01, 0.1, 1, 10 and 100 μM) of four different compounds, appropriate dilutions of a cytotoxic standard and control (untreated) wells. The compounds are dissolved in DMSO to make 20 mM stock solutions on the day of drug addition and freeze at −20° C. Serial dilutions of these 20 mM stock solutions in complete growth medium are made such that 100 μl of these drug solutions in medium of final concentrations equaling 0.01, 0.1, 1, 10 and 100 μM can be added to the cells in triplicate. Standard drugs whose anti-cancer activity has been demonstrated are doxorubicin and SAHA.

After 24 hours from seeding the cells, 10 μL of 3-(4,5-Dimethyl-2-thiazolyl)-2,5-diphenyl-2H-tetrazolium (MTT) solution per well is added and these are then incubated for 3 hours at 37° C., 5% CO₂, 95% air and 100% relative humidity, protected from light. The cells incubated with compounds for 48 hours are treated similarly except with the addition of 20 μL MTT solution per well and a subsequent incubation under the same conditions. After 3 hours of MTT incubation, the contents are aspirated followed by addition of 150 μl. DMSO per well. The plates are agitated to ensure solution of the formazan crystals in DMSO and absorbance at 570 nm is measured.

The percent growth is calculated for each compound's concentration relative to the control and zero measurement wells (T₀; viability right before compound addition). If a test well's O.D. value is greater than the T₀ measurement for that cell line % Growth=(test−zero)/(control−zero)×100. If a test well's O.D. value is lower than the T₀ measurement for that cell line, then, % Growth=(test−zero)/zero×100. Plotting % growth versus experimental drug concentration, GI₅₀ is the concentration required to decrease % growth by 50%; TGI is the concentration required to decrease % growth by 100% and LC₅₀ is the concentration required to decrease % growth by 150%.

### Example 19

### HDAC Inhibition Assay Using Boc-Lys (Ac)-AMC Substrate

Inhibition of HDAC has been implicated to modulate transcription and to induce apoptosis or differentiation in cancer cells. The fluorometric assay provides a fast and fluorescence based method that eliminates radioactivity, extractions or chromatography, as used in traditional assays. The assay is based on two steps. First, the HDAC fluorometric substrate is incubated, which comprises an acetylated lysine side chain, with a sample containing HDAC activity (Mouse Liver Extract). Deacetylation of the substrate sensitizes the substrate. In the second step, the Trypsin stop solution is treated to produce a fluorophore that can be easily analyzed using fluorescence plate reader.

The assay is run with a total volume of 100 μl in a 96 well black microplate. The mouse liver enzyme is diluted to 1:6 with an HDAC buffer. An enzyme cocktail is made that consists of 10 ul diluted enzyme and 30 ul HDAC buffer. 40 ul of enzyme cocktail is dispensed into each well. 10 ul of different concentrations of inhibitor is added to each well, except the enzyme control well. The plate is preincubated at 30° C. for 5 minutes prior to starting the HDAC reaction by adding 50 ul of HDAC substrate (Boc-Lys (Ac)-AMC Substrate) solution. The plate is then incubated at 30° C. for 30 minutes. 100 ul of Trypsin stop solution is then added to stop the reaction. The plate is then incubated again at 30° C. for 20-30 minutes. The release of AMC is monitored by measuring the fluorescence at excitation wavelength of 365 or 360 nm and emission wavelength of 440 or 460 nm. Buffer and substrate alone kept for blank subtraction. See Dennis Wegener et al, Anal. Biochem, 321, 2003, 202-208.

### Example 20

### Test for Induction of Differentiation in A2780 Cells

Increase of alkaline phosphatase (ALP) activity is known as an indicator for differentiation of human colon cancer cells. For example, sodium butylate may increase ALP activity. See, e.g., Young et al., Cancer Res., 45, 2976 (1985) and Morita et al., Cancer Res., 42, 4540 (1982). Thus, differentiation inducing action may be evaluated using ALP activity as an indicator.

To each well of a 96-well plate, 0.1 mL of A2780 cells (15,000 cells/well) is placed. The next day, 0.1 mL of a sequential dilute of test solution with the medium is added. The cells are then incubated for 3 days and the cells on the plate are washed twice with a TBS buffer (20 mM Tris, 137 mM NaCl, pH 7.6). Then, to each well 0.05 mL of 0.6 mg/mL p-nitrophenylphosphate (9.6% diethanolamine, 0.5 mM MgCl.sub.2 (pH 9.6)) solution is added and the solution is then incubated at room temperature for 30 min. The reaction is then quenched with 0.05 mL/well of 3N aqueous sodium hydroxide. For each well, the absorbance at 405 nm is measured to determine the minimum concentration of the compound inducing increase of ALP activity (ALPmin).

### Antitumor Test Procedure

Intraperitoneally inoculated murine myeloid leukemia cells WEHI-3 (1 to 3×10.sup.6 cells) to a Balb/C mouse and oral administration of a compound described herein is initiated on the next day as Day 1. The compound is subsequently orally administered once a day during Days 1 to 4 and Days 7 to 11. The survival days are observed after inoculation and the ratio of the survival days for the calculated and compared to a control group (T/C, %).

### Antitumor Action Test

Inoculate subcutaneously to a nude mouse tumor cells subcultured in a nude mouse (HT-29, KB-3-1). When the volume becomes about 20 to 100 mm³, initiate oral administration of the drug as Day 1. Subsequently orally administer the drug during Days 1 to 5, Days 8 to 12, Days 15 to 19, and Days 22 to 26. The volume of the tumor is determined from the following equation:

(Volume of tumor)=½×(major axis)×(minor axis)².

### Example 21

### Phase I/II Human Clinical Trial of the Safety and Efficacy

Purpose: To study the safety and best dose of an HDAC modulator described herein when given together with azacitidine and compare the efficacy to treatment by azacitidine alone for patients with cancerous diseases (e.g. myelodysplastic syndromes (MDS), chronic myelomonocytic leukemia (CML), or acute myeloid leukemia (AML)).

Patients:

The patients are to be 18 years of age or above, with a performance status of 0-2 (ECOG) and a life expectancy of at least 6 months. They should have normal levels of hemoglobin (≧8 g/dL, transfusion allowed; no disseminated intravascular coagulation), bilirubin (unless due to hemolysis or Gilbert's syndrome), aspartate transaminase and alanine transaminas (≦2.5 times the upper limit of normal), and creatinine (or with a creatinine clearance of ≧60 mL/min). Patients are not to be pregnant or nursing, negative pregnancy test, fertile patients must use effective contraception during and for 3 months after study treatment, no untreated active infection, no other serious or uncontrolled medical condition, and no known hypersensitivity to the administered drugs.

Patients are to be diagnosed via bone marrow aspiration and/or biopsy with a cancerous disease (non-therapy induced), such as MDS (any IPSS score allowed except low score only allowed in Phase I for patients with absolute neutrophil count <1,000/mm3, untransfused hemoglobin <8 g/dL, platelet count <20,000/mm³, or anemia requiring transfusion; for Phase II patients with low or intermediate-1 IPSS must have platelet count <50,000/mm³ and/or absolute neutrophil count <500/mm³), CML (WBC<12,000/mm³ measured twice within past 4 weeks, 2 weeks apart), or AML (for Phase I, relapsed or refractory disease, WBC<30,000/mm³ for ≧2 weeks before study entry, acute promyelocytic leukemia allowed if patient is in at least second relapse and has already received treatment regimens containing arsenic trioxide and isotretinoin, or untreated AML allowed provided patient meets one or more of the following criteria: age 60 and over; AML arising in the setting of an antecedent hematologic disorder; high-risk cytogenetic abnormalities; medical conditions that may compromise the ability to give cytotoxic chemotherapy as the primary modality; or refused cytotoxic chemotherapy; for Phase II, refractory anemia with excess blasts in transformation by FAB criteria allowed, AML-TLD by WHO criteria allowed in patients with no history of antecedent hematologic disorder, and WBC≦30,000/mm³ measured twice within the past 4 weeks, 2 weeks apart, and WBC that has doubled over 4 weeks and >20,000/mm³ is not eligible). The patients are not to have clinical evidence of CNS, pulmonary leukostasis, or CNS leukemia.

Patients are limited to prior or concurrent therapies of the following: more than 3 weeks since prior hematopoietic growth factors; none or at least 3 weeks since prior hydroxyurea (2 weeks for AML) and no concurrent hydroxyurea; recovered from all prior therapies; at least 2 weeks since prior cytotoxic therapy (AML patients); more than 3 weeks since other prior therapy; no other concurrent investigational or commercial agents or therapies; no concurrent valproic acid, epoetin alfa, or darbepoetin alfa; no filgrastim or pegfilgrastim during days 1-10 of each treatment course. All studies are to be performed with institutional ethics committee approval and patient consent.

Study Design:

Phase I: This is a multicenter, dose-escalation study of the HDAC modulator. Patients receive azacitidine subcutaneously on days 1-10 and the HDAC inhibitor orally on days 3 and 10. Courses repeat every 28 days in the absence of disease progression or unacceptable toxicity. Cohorts of 3-6 patients receive escalating doses of the HDAC modulator until the maximum tolerated dose (MTD) is determined. Patients who do not achieve hematologic improvement or partial or complete response but who have stable disease after 4 courses of therapy may receive an additional 4 courses of therapy at a higher dose than what is originally assigned. Patients receive adjusted doses of azacitidine based on clinical response. The MTD is defined as the dose preceding that at which 2 of 3 or 2 of 6 patients experience dose-limiting toxicity. Up to 9 additional patients are treated at the MTD. Observe the safety and toxicity of the HDAC modulator in combination with azacitidine, the response rate measured by International Working Group (IWG) criteria, optimal dose combination, and correlation between HDAC modulator pharmacokinetics with clinical and molecular outcomes measured by standard methods (e.g. Cmax, AUC, H2AX gamma induction, histone acetylation, and promoter methylation reversal).

Phase II: This is a randomized, multicenter study. Patients are stratified according to disease (e.g. MDS high/intermediate-2 vs. MDS low/intermediate-1 vs. CML vs. AML with multilineage dysplasia). Patients are randomized to 1 of 2 treatment groups. For group I, patients receive azacitidine subcutaneously once daily on days 1-10. For group II, patients receive azacitidine subcutaneously as in group I and the HDAC modulator orally on days 3 and 10. Treatment in both groups are repeated every 28 days for at least 6 and up to 24 courses in the absence of disease progression or unacceptable toxicity. After completion of study treatment, patients are followed periodically for 5 years. Compare the overall response rate (complete, partial, triliniage, and hematologic improvement-major by IWG criteria) in patients treated with azacitidine with vs. without the HDAC modulator. Also compare the major response rate (complete and partial responses by IWG criteria) in patients treated with these regimens. Evaluate the toxicity of these regimens. Identify changes in gene promoter methylation and gene expression that may be associated with these regimens. Identify other molecular mechanisms (such as DNA damage) that may be associated with response to these regimens.

One of skill in the art will readily recognize that this study protocol can be used for other combinations of the HDAC modulators described herein when given together with drugs other than azacitidine for the treatment of other cancerous and non-cancerous diseases.

### Example 22

### Treatment of Hodgkin's Lymphoma

A patient with relapsed or refractory Hodgkin's Lymphoma is administered 2-4 mg/m² of a compound of Formula I on days 3 and day 10. Treatment with the compound of Formula I is repeated every 28 days for at least 6 and up to 24 courses in the absence of unacceptable toxicity.

### Example 23

### Treatment of Non-Hodgkin's Lymphoma

A patient diagnosed with non-hodgkin's lymphoma is administered 2-4 mg/m² of a compound of Formula I on days 3 and day 10. Treatment with the compound of Formula I is repeated every 28 days for at least 6 and up to 24 courses in the absence of unacceptable toxicity.

### Example 24

### Treatment of Glioblastoma After Radiotherapy

A patient diagnosed with glioblastoma undergoes conventional radiotherapy once daily, 5 days a week, for 6 weeks. During this time, the patient is concomitantly administered 2-4 mg/m² of a compound of Formula I on days 3 and day 10. Treatment with the compound of Formula I is repeated every 28 days during radiation treatment in the absence of unacceptable toxicity.

### Example 25

### Treatment of Melanoma (IL-2 Combination Therapy)

A patient diagnosed with melanoma is administered high-dose bolus IL-2 (720 000 IU/Kg) intravenously every 8 hours as tolerated but not to exceed 15 doses. During this time, the patient is also administered 2-4 mg/m² of a compound of Formula I on days 3 and day 10. Treatment with the compound of Formula I is repeated every 28 days during radiation treatment in the absence of unacceptable toxicity.

### Example 26

### Treatment of Renal Cell Cancer (IL-2 Combination Therapy)

A patient diagnosed with renal cell cancer is administered high-dose bolus IL-2 (720 000 IU/Kg) intravenously every 8 hours as tolerated but not to exceed 15 doses. During this time, the patient is also administered 2-4 mg/m² of a compound of Formula I on days 3 and day 10. Treatment with the compound of Formula I is repeated every 28 days during radiation treatment in the absence of unacceptable toxicity.

### Example 27

### Treatment of Prostate Cancer (13-Cis Retinoic Acid Combination Therapy)

A patient diagnosed with prostate cancer receives oral 13-cis Retinoic Acid at a dose of 1.0 mg/kg/day, given as a single daily dose and rounded to the nearest 10 mg, for a period of 12 months. The 13-cis Retinoic Acid is provided in the form of soft gelatin capsule of 10, 20 or 40 mg. On days 3 and 10, the patient also receives 2-4 mg/m² of a compound of Formula I. Treatment with the compound of Formula I is repeated every 28 days for at least 6 and up to 24 courses in the absence of unacceptable toxicity.

### Example 28

### Treatment of Non-Small Cell Lung Cancer (Erlotinib Combination Therapy)

A patient diagnosed with non-small cell lung cancer is administered 100-150 mg/day of erlotinib for three weeks and 2-4 mg/m² of a compound of Formula I on days 3 and 10. This treatment is repeated every 28 days for at least 6 and up to 24 courses in the absence of unacceptable toxicity.

### Example 29

### Treatment of AML (ATRA Combination Therapy)

A patient diagnosed with AML is administered 45 mg/m2 ATRA daily and 2-4 mg/m² of a compound of Formula I on days 3 and day 10. Treatment with the compound of Formula I is repeated every 28 days for at least 6 and up to 24 courses in the absence of unacceptable toxicity.

### Example 30

### Treatment of AML (Anti-Estrogen Combination Therapy)

A patient diagnosed with AML is administered 200-700 mg/day p.o. for 7 days in combination 2-4 mg/m² of a compound of Formula I on day 3 and day 10. Courses are repeated every 21 days in the absence of disease progression or unacceptable toxicity.

### Example 31

### Treatment of AML (Decitabine Combination Therapy)

A patient diagnosed with AML is administered 15-20 mg/m²/IV over 1 hr daily for 10 days and 2-4 ng/m² of a Compound of Formula I on day 3 and 10. Courses are repeated every 21 days in the absence of disease progression or unacceptable toxicity.

### Example 32

### Treatment of Solid Tumors (Decitabine Combination Therapy)

A patient with a solid tumor is administered 600 mg/m² decitabine IV over 1 hour on days 1-5 and 2-4 mg/m² of a Compound of Formula I on day 3. Courses are repeated every 21 days in the absence of disease progression or unacceptable toxicity.

### Syndax Examples

### Example 33

### Histone Deacetylase Inhibition Ameliorates Neurodegenerative Phenotype in Huntington's Disease Mice

Purpose: Huntington's disease is a progressive neurodegenerative disorder caused by the CAG repeat in the gene coding for the protein, huntingtin. Mutant huntingtin has been shown to alter expression of other genes. A strategy for treatment of Huntington's disease is to modulate the regulation of gene expression via the use of inhibitors of histone deacetylases (HDAC) described herein.

Animals

Male mice of R6/2 strain (The Jackson Laboratory) are transgenic mouse models for Huntington disease (Ferrante E J et al., J. Neurosci., 2003, 23(28):9418-27) and are bred with females of wild-type background. Offspring are genetically identified as R6/2 or wild-type by PCR genotyping DNA obtained from their tails and the litters subsequently randomized for treatment with HDAC or control. The animals are kept on a 12 h light/dark cycle and food and water are provided ad libitum. Animal care is performed in accordance to the NIH Guide for the Care and Use of Laboratory Animals.

Dosing Regimen

At age 20 d, a dose-response study is performed, treating groups of wild-type mice (n=40) and R6/2 mice (n=40) with 100, 200, 400, 600, 1000, 1500, 10000 mg/kg daily intraperitoneal injection (1000) of HDAC inhibitor dissolved in PBS. Control groups are given PBS injections or left untreated.

Clinical Assessment:

Motor performance is assessed by rotarod apparatus (Columbus Instruments). Mice are acclimated on the apparatus at days 21 and 22. At ages 23 to death, HDAC treated and untreated wild-type and R6/2 mice are assessed weekly for motor performance on the rotarod. Three 60 second trials are given during a session and recorded. Body weights are also recorded the same day of motor performance.

Survival

R6/2 mice are assessed daily for morbidity and mortality. Euthanization occurs when the R6/2 Huntington Disease mice are unable to right themselves after being placed on their back.

Acetylated Histone Quantitation Assay

At day 60, 90, and 120, a group of wild-type and R6/2 mice are sacrificed and their brains frozen, weighed, and tissue-sheared by mortar and pestle. Powdered brain tissue is lysed in 1% Triton-based cell lysis buffer for extracting proteins. Equal concentrations of lysates are separated by SDS-PAGE electrophorhesis and acetylated histone H3 and H4 are assessed by Western blotting with antibodies specific to acetylated histone H3 and H4.

Histopathological Analysis of HDAC Inhibitor Neuroprotection

At day 60, 90, and 120, a group of wild-type and R6/2 mice are sacrificed. Brains are obtained and fixed with freshly prepared 4% buffered formaldehyde. Brains are serial sliced into coronal serial step sections from the rostral neostriatum through the level of the anterior commissure and immunostained for aggregation of huntingtin protein. Neuronal atropy can also be assessed visually with the serial step sections.

### Example 34

### Histone Deacetylase Inhibition Exhibits Anti-Inflammatory Properties on Arthritis in Mice

Purpose: Rheumatoid arthritis (RA) is a chronic inflammatory disease that affects the joints of hands and feet and is thought of an autoimmune disease. The use of inhibitors of histone deacetylases described herein can reduce and downregulate production of proinflammatory cytokines, immune stimulators, and nitric oxide, a contributor in inflammatory diseases.

Animals:

DBA/1J mice (male, 8-weeks old, The Jackson Laboratory) are kept on a 12 h light/dark cycle. Food and water are provided ad libitum. Animal care is performed in accordance to the NIH Guide for the Care and Use of Laboratory Animals.

Collagen immunization to induce arthritis.

Bovine collagen type II is prepared (2 mg/ml) is prepared with 0.05 M acetic acid at 4° C. Prior to immunization, equal volumes of collagen solution are mixed with adjuvant (complete Freund's adjuvant) by a homogenizer under an ice-water bath. 0.1 ml of the homogenate solution is injected intradermally at the base of the tail. 29 d after immunization, a booster injection of lipopolysaccharide (0.4 mg/ml saline) is given intraperitoneally.

Experimental Design:

Mice (n=50) are divided into 5 groups. Groups 1-4 are immunized with collagen with Group 5 left untreated. Group 1 is treated with a control vehicle (0.1 ml 5% DMSO subcutaneous daily); Group 2 is treated with a high dose of HDACi (50 mg/kg subcutaneous daily); Group 3 is treated with a low dose of HDACi (5 mg/kg subcutaneous aily); group 4 is treated with methotraxate, a standard therapeutic agent for RA (0.1 mg/kg subcutaneous daily). Daily treatment is given for 43 days. Day 40, 0.2 ml blood is collected by retro-orbital puncture under general anesthesia. Day 43, all mice are sacrificed and hind paws removed for X-ray analysis and histological examination. Body weights are recorded weekly.

Arthritis Analysis:

Arthritis is graded on a 0-4 score method as described (Nishida K et al., Arthritis Rheum., 2004, 10:3365-3376). Briefly, 0, no symptoms; 1, mild with redness and swelling of one joint type; 2, moderate redness and swelling of two or more joints; 3, severe redness and swelling of the entire paw; 4, maximum swelling and redness, entire limb is inflamed.

X-Ray Analysis of Bone Erosion:

X-ray photography is taken of the hind paws of the mice. Bone erosion is scored on a 0-5 scale as follows: 0, normal intact bone outlines; 1, slight abnormality of 1-2 exterior metatarsal bones with little bone erosion; 2, definite abnormality of 3-5 exterior metatarsal bones with bone erosion; 3, medium destructive abnormality with all exterior metatarsal bones and major erosion; 4, severe destructive abnormality with all metatarsal bones showing complete erosion; and 5, mutilating abnormality with no bony outlines.

Pro-Inflammatory Cytokine Assays:

Blood drawn by retro-orbital puncture of the mice at day 40 is assessed for serum IL-1β and IL-6 levels by enzyme-linked immunosorbent assay using protocols supplied by manufacturer (Biosource, Camarillo, Calif.).

### Example 35

### Histone Deacetylase Inhibition Heals Wounds

Clinical Trial of the Safety and/or Efficacy of a Compound of Formula I in Treatment of Wound Healing by administration of a compound of Formula I is described herein.

Objective: To evaluate the safety and best dose of a topically administered treatment containing a Compound of Formula I described herein for prevention of radiation induced skin damage, and promotion wound healing

Study subjects are adult female Sprague Dawley (SD) rats weighing 250-300 g at the time of irradiation. Each rat is caged alone and allowed chow and water. Prior to irradiation the skin over the gluteal area is shaved completely and radiation fields with 2 cm diameter is outlined with a marking pen. Each rat is anesthetized with pentobarbital 50 mg/kg i.p. prior to irradiation. Irradiation is administered using an electron beam with 6 MeV energy produced by a linear accelerator. At Day 0 a dose at 4Gy/min-40Gy/min is administered to the prepared area.

The study subjects are divided into three subgroups: one subgroup treated with skin irradiation followed by vehicle, another with skin irradiation followed by a treatment containing a Compound of Formula I described herein, and a third with skin irradiation only. Thereafter, vaseline (negative control), madecassol (positive control), or vehicle is applied topically at a dose of 200 mg/irradiated skin surface twice per day from Day 1 through Day 90.

Acute skin reactions are evaluated and scored through 90 days after irradiation using a modified skin score system as follows: 0, normal; 0.5, slight epilation; 1.0, epilation in about 50% of the radiated area; 1.5, epilation in more than 50% of the area; 2.0, complete epilation; 2.5, dry desquamation in more than 50% of the area; 3.0, moist desquamation in a small area; and 3.5, moist desquamation in most of the area. The mean of skin scores from five samples in the same group is evaluated.

III. Pharmaceutical Compositions

### Example 36

### Parenteral Composition

An i.v. solution is prepared in a sterile isotonic solution of water for injection and sodium chloride (˜300 mOsm) at pH 11.2 with a buffer capacity of 0.006 mol/l/pH unit. The protocol for preparation of 100 ml of a 5 mg/ml a compound of Formula I-VIII for i.v. infusion is as follows: add 25 ml of NaOH (0.25 N) to 0.5 g of a compound of Formula I-VIII and stir until dissolved without heating. Add 25 ml of water for injection and 0.55 g of NaCl and stir until dissolved. Add 0.1N HCl slowly until the pH of the solution is 11.2. The volume is adjusted to 100 ml. The pH is checked and maintained between 11.0 and 11.2. The solution is subsequently sterilized by filtration through a cellulose acetate (0.22 μm) filter before administration.

### Example 37

### Oral Composition

To prepare a pharmaceutical composition for oral delivery, 100 mg of a compound of Formula I-VIII is mixed with 750 mg of a starch. The mixture is incorporated into an oral dosage unit, such as a hard geletin capsule or coated tablet, which is suitable for oral administration.

Many modifications, equivalents, and variations of the present invention are possible in light of the above teachings, therefore, it is to be understood that within the scope of the appended claims, the invention may be practiced other than as specifically described.

1. A compound of Formula I: 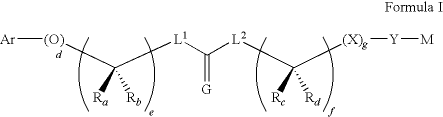
or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 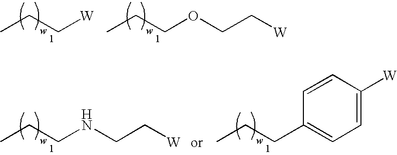
where W is selected from: 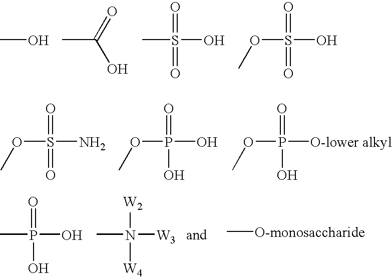
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 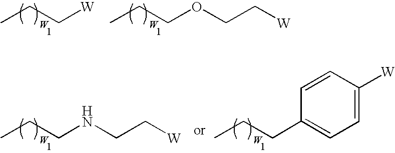
where W is selected from: 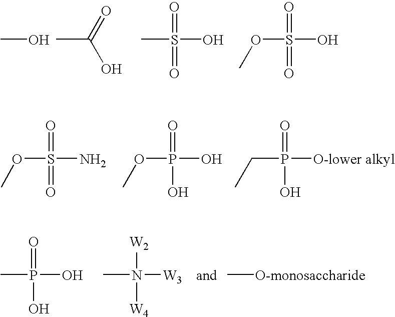
where W₁ is 0, 1, 2 or 3; W₂ and W₃ are each independently hydrogen or methyl or, when taken together with the nitrogen to which they are attached, W₁ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂;
X is 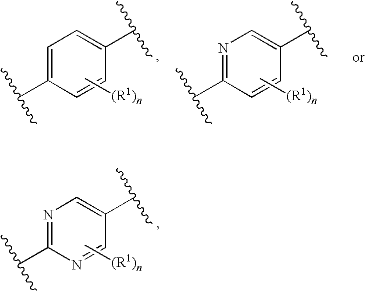
wherein each R¹ is independently halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂,
n is 0, 1, 2, 3 or 4, and the water solubilizing group is: 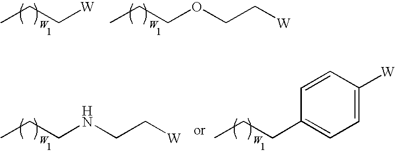
where W is selected from: 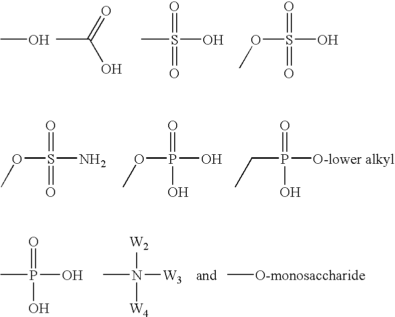
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from 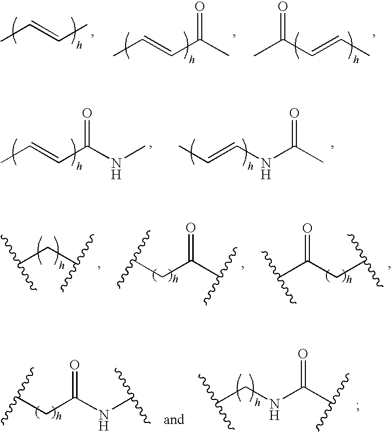
wherein h is 0, 1, 2, 3 or 4;
M is selected from: 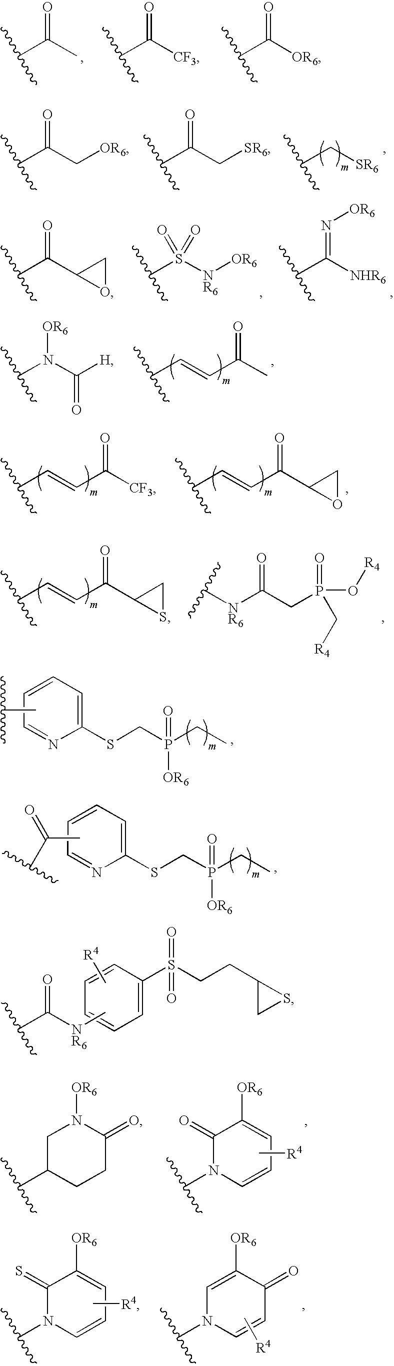 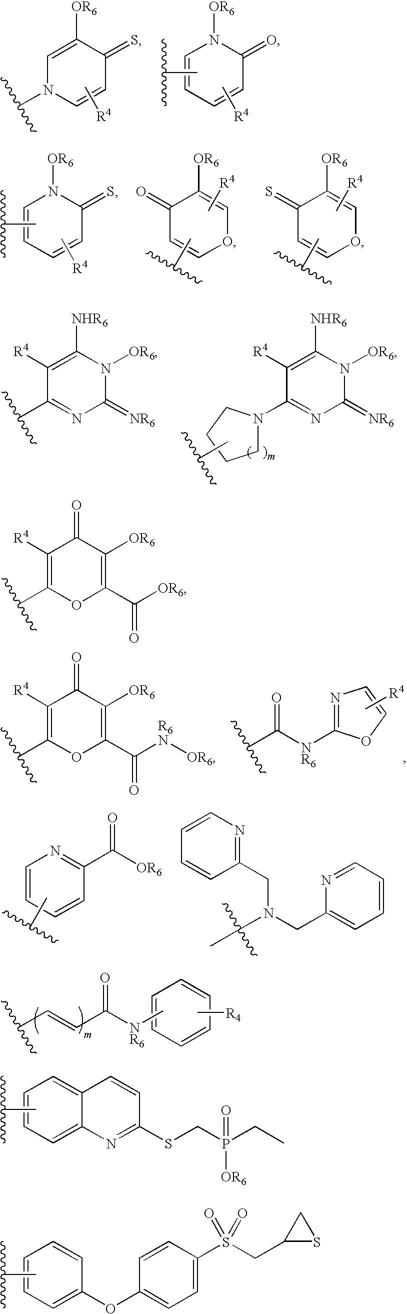 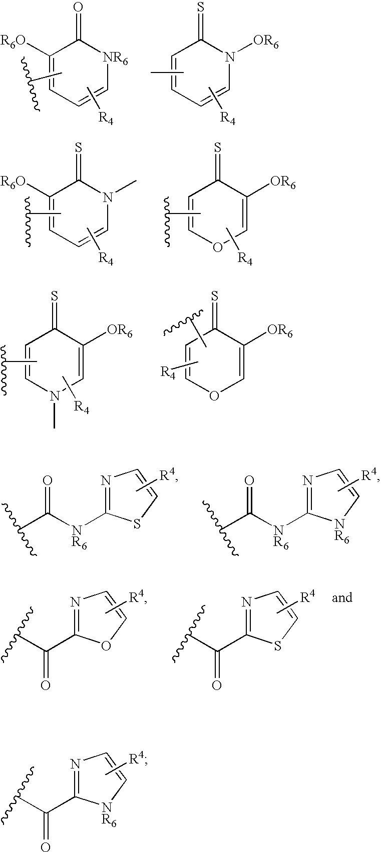
wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is: 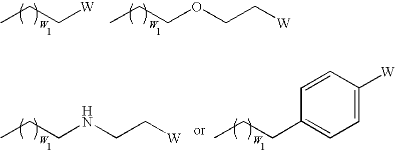
where W is selected from: 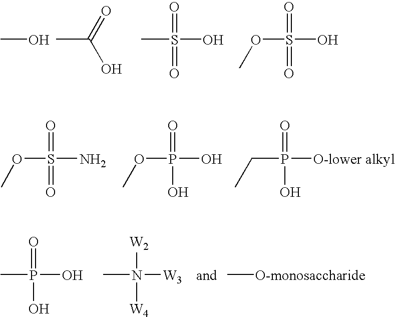
where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom; and
R⁶ is hydrogen, lower alkyl, lower acyl, lower alkoxycarbonyl, carbamoyl, —PO₃H₂, or —SO₃H; and
m=0 or 1 or 2,
provided that when g is 1 and Y is amidomethyl or amidoethyl, then Y is not carboxyl or ethoxycarbonyl;
when g is 1 and Y is a bond, then M is not carboxyl; and that the compound is not:

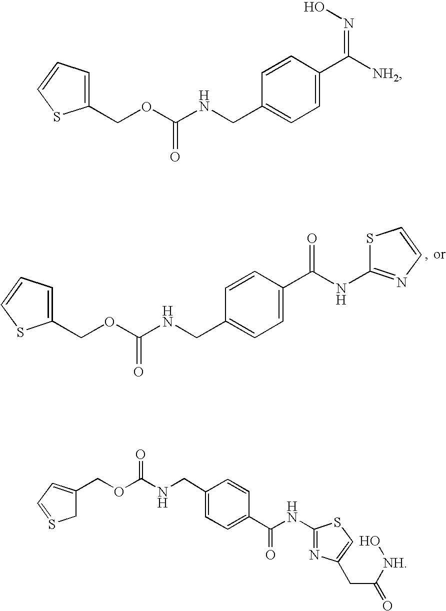

2. (canceled)

3. The compound of claim 1, wherein Ar is an C₅-C₁₅ aryl or C₅-C₁₅ heteroaryl group, wherein said aryl or heteroaryl groups are optionally substituted with 1-3 substituents each independently selected from hydrogen, halogen, hydroxy, amino, carboxy, C₁-C₄ alkyl, C₁-C₄ alkoxy, C₁-C₄ aminoalkyl, C₁-C₄ alkylamino, C₁-C₄ alkylthio, C₁-C₄ haloalkyl, C₁-C₄ perfluoroalkyl, C₁-C₄ perfluoroalkoxy and C₁-C₄ alkoxycarbonyl.

4. The compound of claim 1, wherein g is I and each R¹ is independently halogen, hydroxy, or a substituted or unsubstituted group selected from amino, carboxy, C₁-C₄ alkyl, C₂-C₅ alkenyl, C₂-C₅ alkynyl, C₁-C₄ alkoxy, C₁-C₄ aminoalkyl, C₁-C₄ alkylamino, C₁-C₄ alkylthio, C₁-C₄ perfluoroalkyl, C₁-C₄ perfluoroalkoxy or C₁-C₄ alkoxycarbonyl; and n is 0, 1 or 2.

5. (canceled)

6. The compound of claim 1, wherein Y is

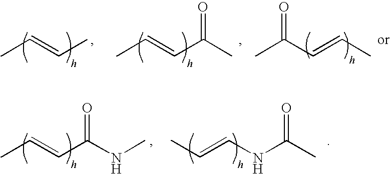

7. The compound of claim 1, wherein Y is

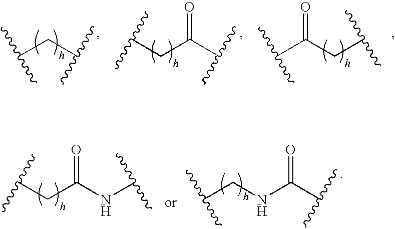

8. (canceled)

9. The compound of claim 1, wherein n is 0.

10-20. (canceled)

21. The compound of claim 1, wherein Ar is:

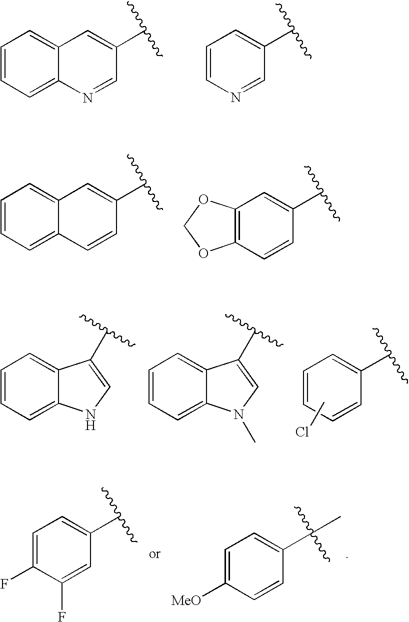

22-24. (canceled)

25. The compound of claim 1, wherein Ar is an optionally substituted C₆-C₁₀ aryl or optionally substituted C₅-C₉ heteroaryl group.

26. The compound of claim 1, wherein L¹ and L² are independently —O—, —N(R³)—, wherein R³ is hydrogen or a water solubilizing group.

27. (canceled)

28. The compound of claim 1, wherein R¹ is selected from hydrogen, alkyl, alkoxy, halogen, —CN, hydroxy and a water solubilizing group.

29. The compound of claim 1, wherein Y is selected from 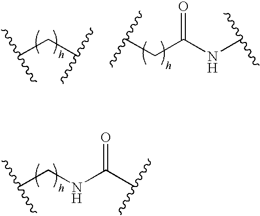
where h is 0 or 1 or 2.

30-47. (canceled)

48. A compound of claim 1 according to Formula 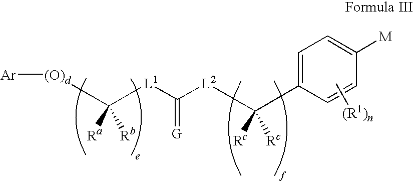
or a pharmaceutically acceptable salt, prodrug, solvate, polymorph, tautomer or isomer thereof.

49. The compound or a pharmaceutically acceptable salt of claim 48.

50-59. (canceled)

60. A pharmaceutical composition comprising a compound of Formula I: 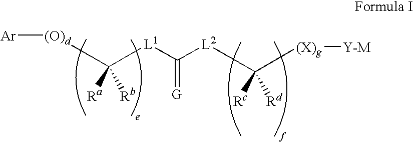
or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 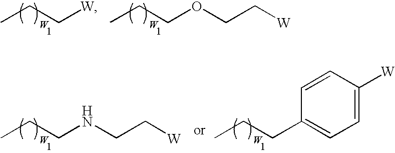
where W is selected from: 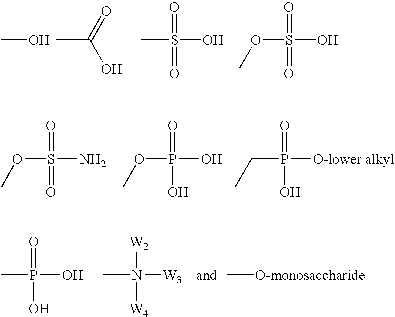
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 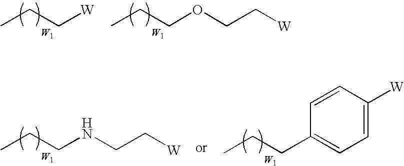
where W is selected from: 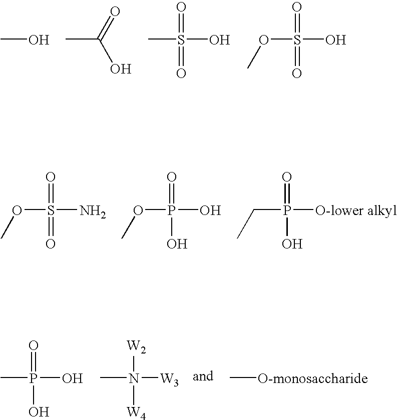
where W₁ is 0, 1, 2 or 3; W₂ and W₃ are each independently hydrogen or methyl or, when taken together with the nitrogen to which they are attached, W₁ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl,
wherein L is a bond, —C(O)—, —S(O), and —S(O)₂; 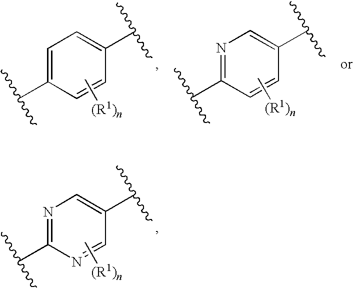
X is
wherein each R¹ is independently halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂,
n is 0, 1, 2, 3 or 4, and the water solubilizing group is: 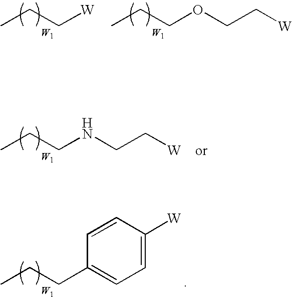
where W is selected from: 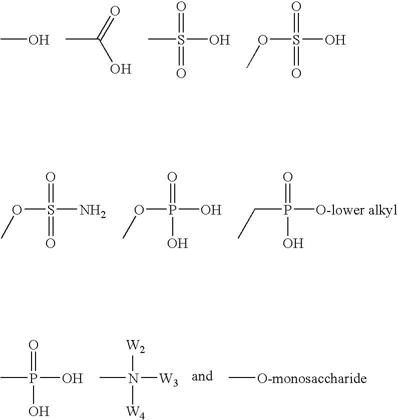
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from 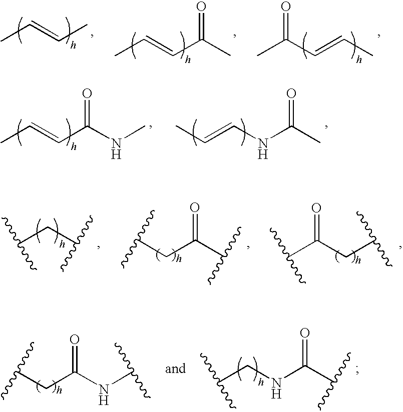
wherein h is 0, 1, 2, 3 or 4;
M is selected from: 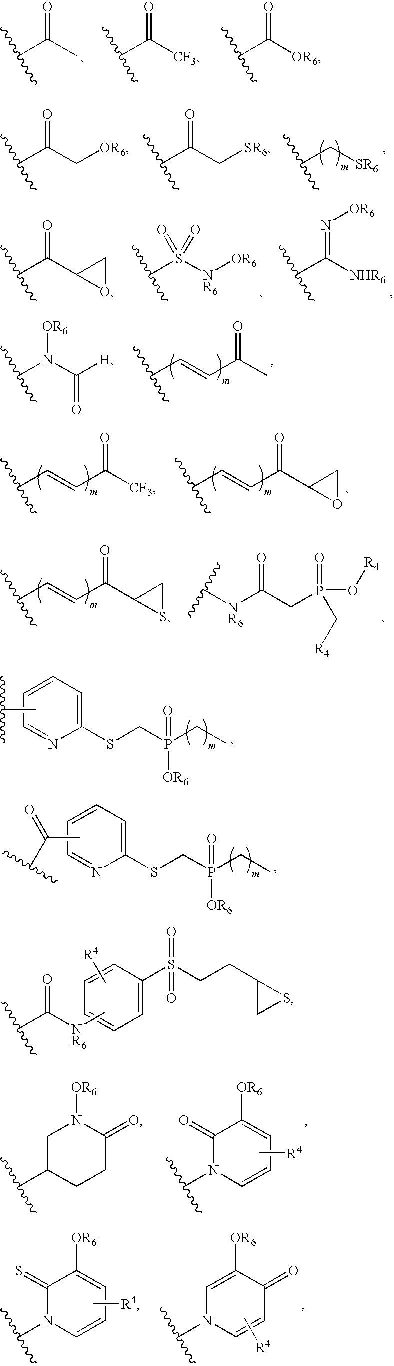 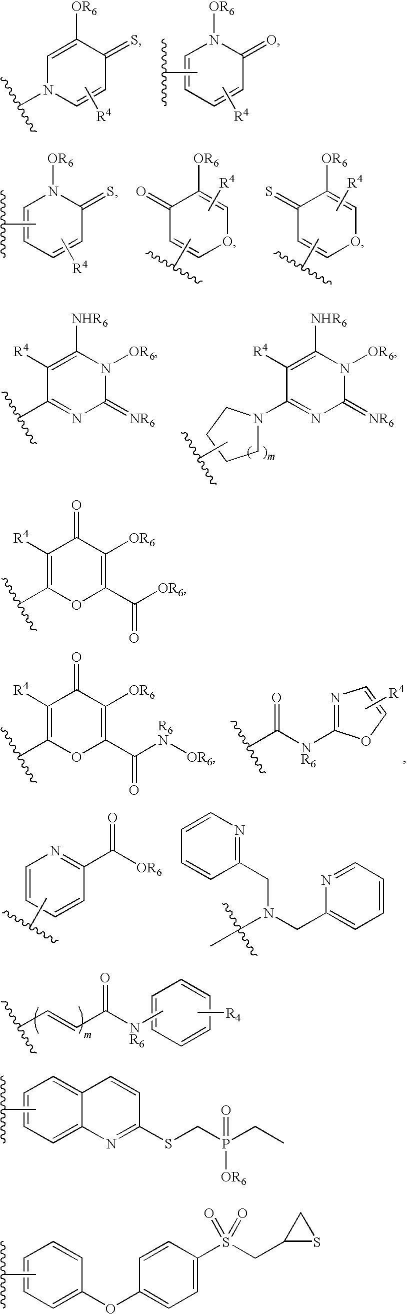 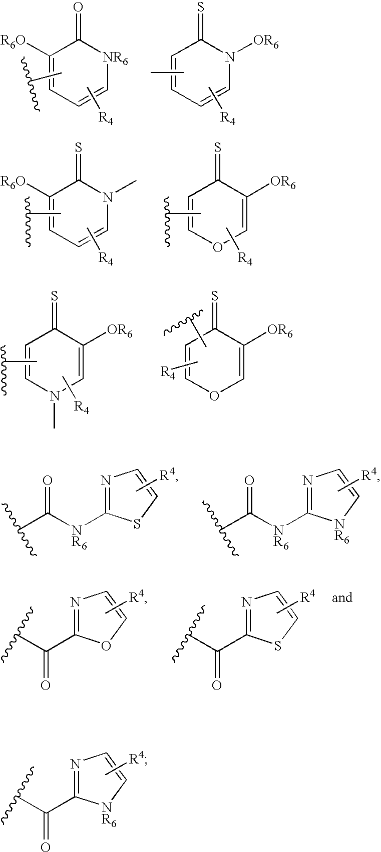
wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is: 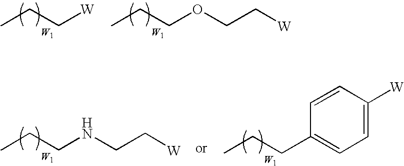
where W is selected from: 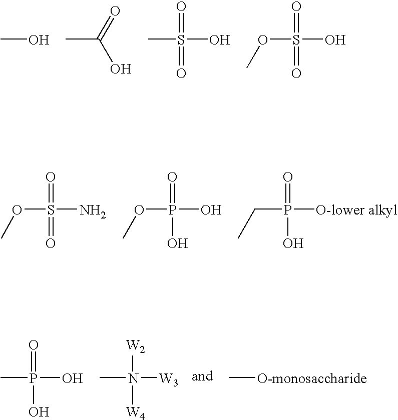
where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom; and
R⁶ is hydrogen, lower alkyl, lower acyl, lower alkoxycarbonyl, carbamoyl, —PO₃H₂, or —SO₃H; and
m=0 or 1 or 2,
provided that when g is 1 and Y is amidomethyl or amidoethyl, then Y is not carboxyl or ethoxycarbonyl;
when g is 1 and Y is a bond, then M is not carboxyl; and
that the compound is not:

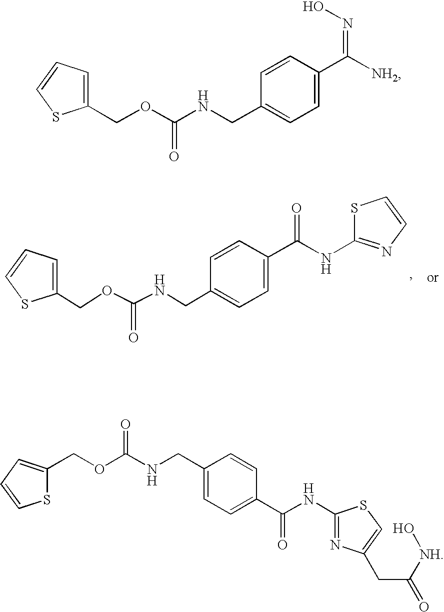

61. The pharmaceutical composition of claim 60, further comprising at least one pharmaceutically acceptable carrier.

62-71. (canceled)

72. A method for degrading, inhibiting the growth of or killing cancer cells comprising contacting the cells with an amount of a composition effective to degrade, inhibit the growth of or kill cancer cells, the composition comprising a compound of Formula I: 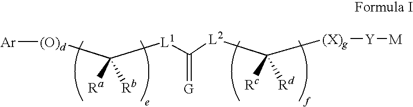
or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 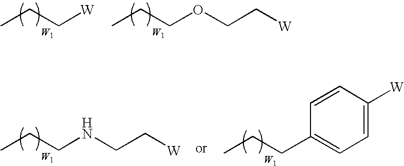
where W is selected from: 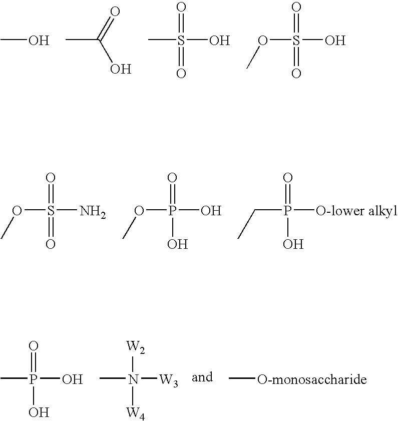
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 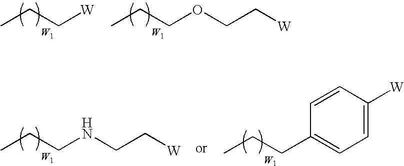
where W is selected from: 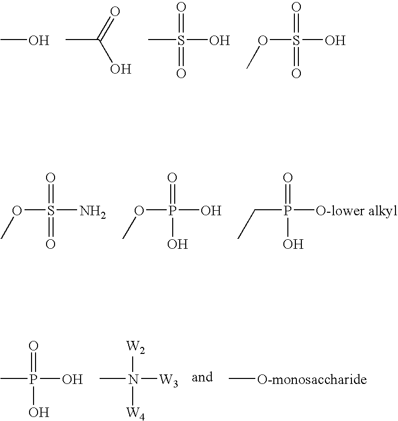
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl,
wherein L is a bond, —C(O)—, —S(O), and —S(O)₂;
X is 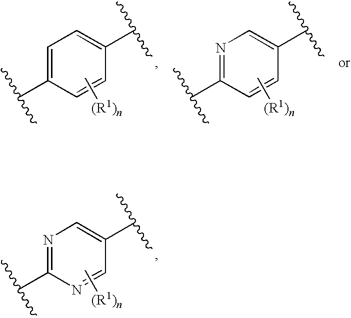
wherein each R¹ is independently halogen, —CN, a water solubilizing group, -L-OH; -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂,
n is 0, 1, 2, 3 or 4, and the water solubilizing group is: 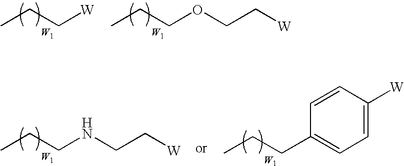
where W is selected from: 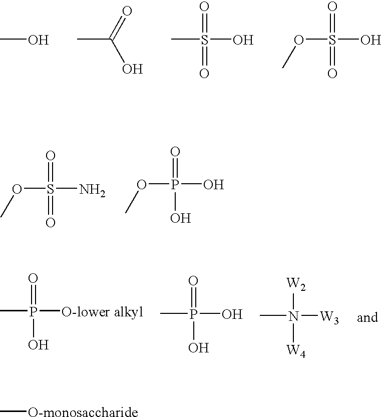
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from 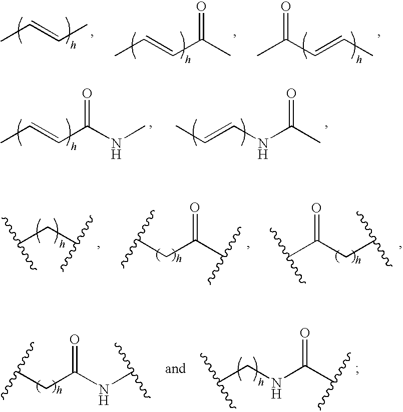
wherein h is 0, 1; 2, 3 or 4;
M is selected from: 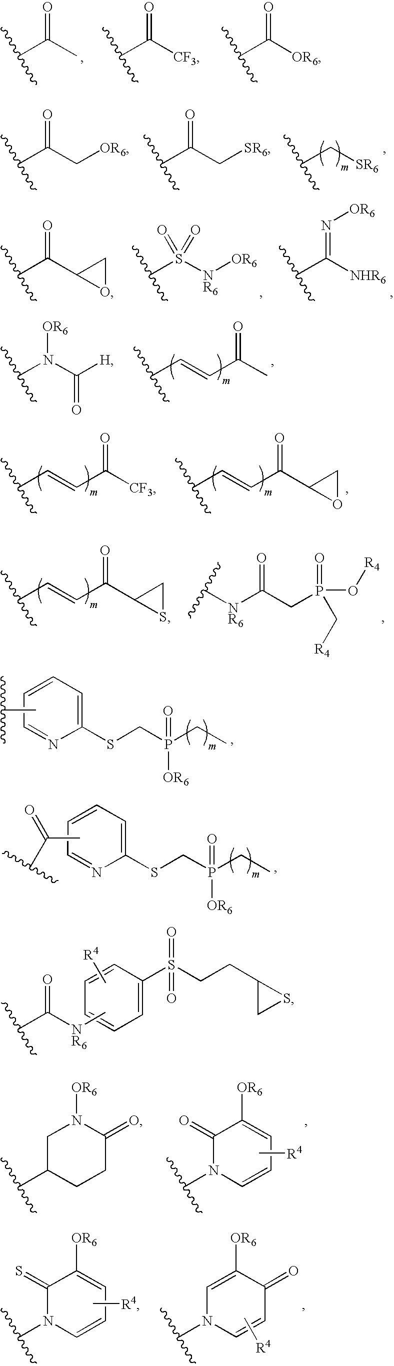 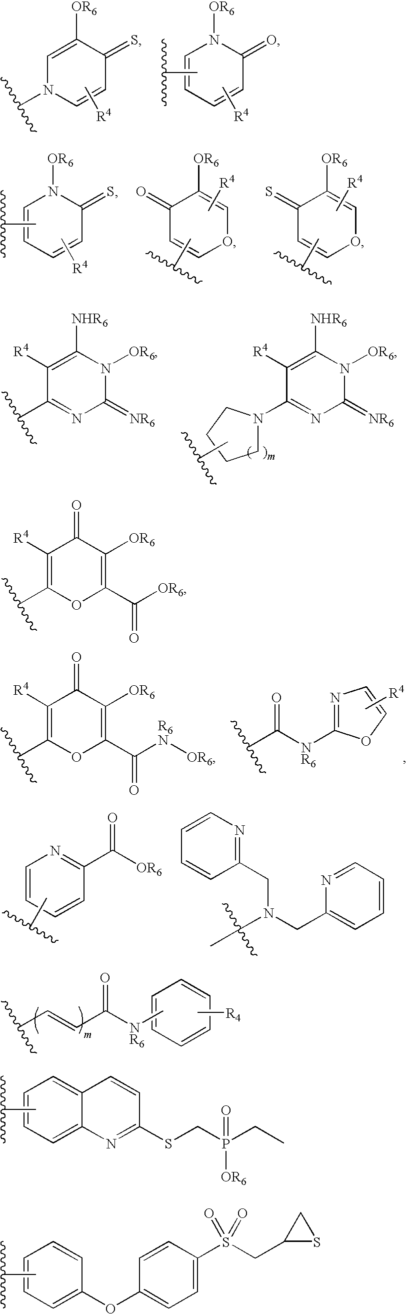 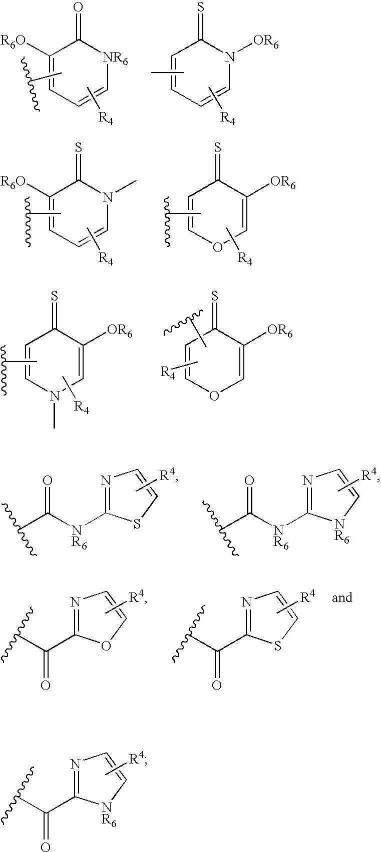
wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is: 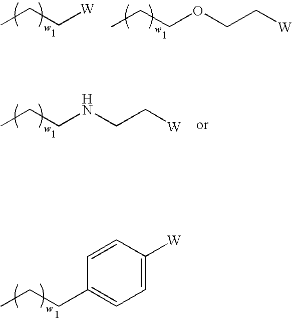
where W is selected from: 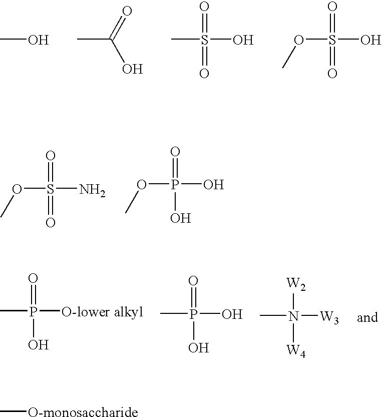
where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom; and
R⁶ is hydrogen, lower alkyl, lower acyl, lower alkoxycarbonyl, carbamoyl, —PO₃H₂, or —SO₃H; and
m=0 or 1 or 2,
provided that the compound is not:

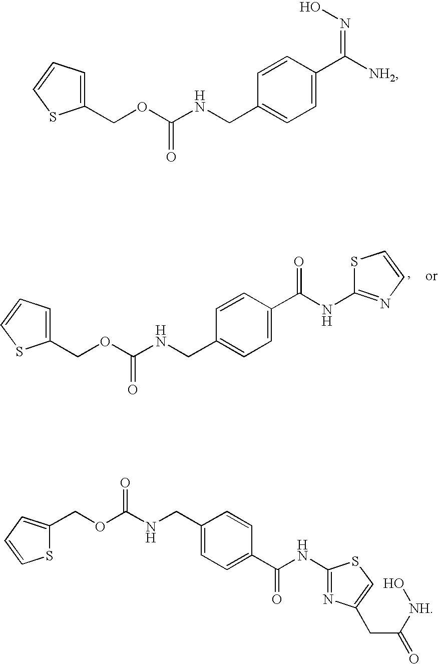

73. The method of claim 72, wherein said cancer cells comprise brain, breast, lung, ovarian, pancreatic, prostate, renal, or colorectal cancer cells.

74. A method of inhibiting tumor size increase, reducing the size of a tumor, reducing tumor proliferation or preventing tumor proliferation in an individual comprising administering to said individual an effective amount of a composition to inhibit tumor size increase, reduce the size of a tumor, reduce tumor proliferation or prevent tumor proliferation, the composition comprising a compound of Formula I: 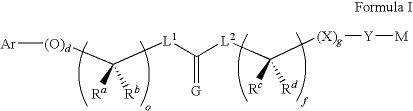
or a pharmaceutically acceptable salt, prodrug, solvate, polymorph, tautomer or isomer thereof, wherein:
Ar is an optionally substituted C₅-C₁₅ aryl or optionally substituted C₅-C₁₅ heteroaryl group;
d is 0 or 1;
e is 0, 1, 2 or 3;
f is 0, 1, 2 or 3;
g is 0 or 1;
L¹ and L² are each independently —O—, —N(R³)—, —ON(R³)—, or —N(R³)O—;
where each R³ is independently hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 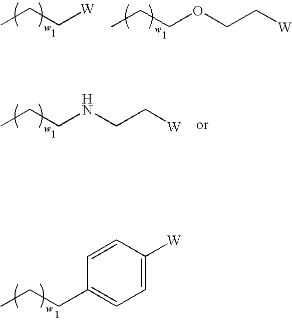
where W is selected from: 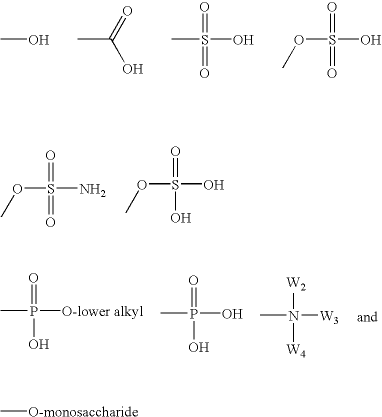
where W₁ is 0, 1, 2 or 3; W₂ and W₃ are each independently hydrogen or methyl or, when taken together with the nitrogen to which they are attached, W₁ and W₃ form a five or six membered ring that optionally contains an oxygen atom or a second nitrogen atom; and W₄ is an electron pair or an oxygen atom;
G is O, S, or NR⁵,
where R⁵ is hydrogen or a substituted or unsubstituted group selected from alkyl, alkenyl, alkynyl, heteroalkyl, alkoxy, cycloalkyl, cycloalkenyl, heterocycloalkyl, aryl, heteroaryl, mercaptoalkyl, haloalkyl, carboxyalkyl and a water solubilizing group, wherein the water solubilizing group is: 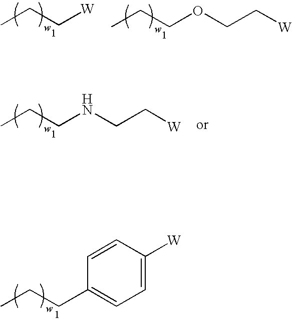
where W is selected from: 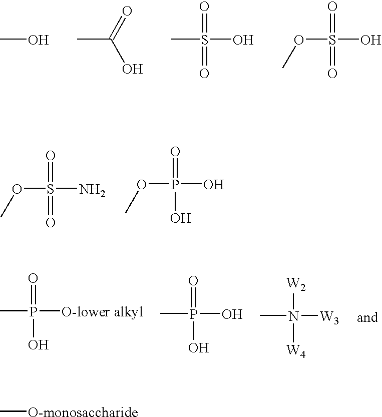
where W₁ is 0, 1, 2 or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
R^{a}, R^{b}, R^{c} and R^{d} are each independently hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂;
X is 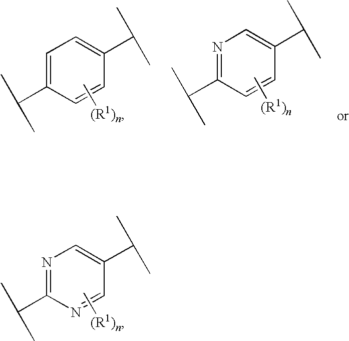
wherein each R¹ is independently halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂,
n is 0, 1, 2, 3 or 4, and the water solubilizing group is: 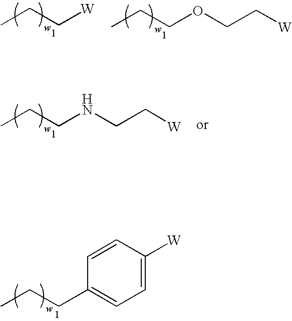
where W is selected from: 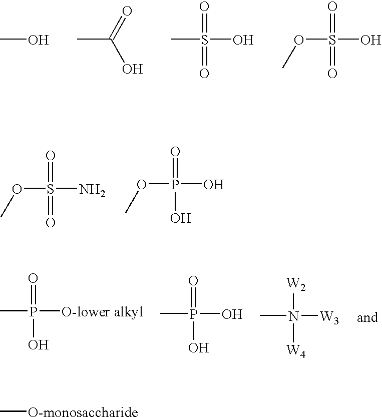
where W₁ is 0, 1, 2 or 3; W₁ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom;
Y is selected from 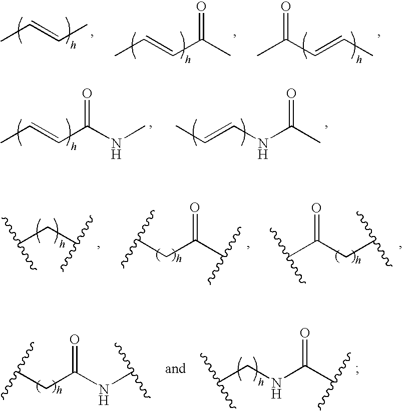
wherein h is 0, 1, 2, 3 or 4;
M is selected from: 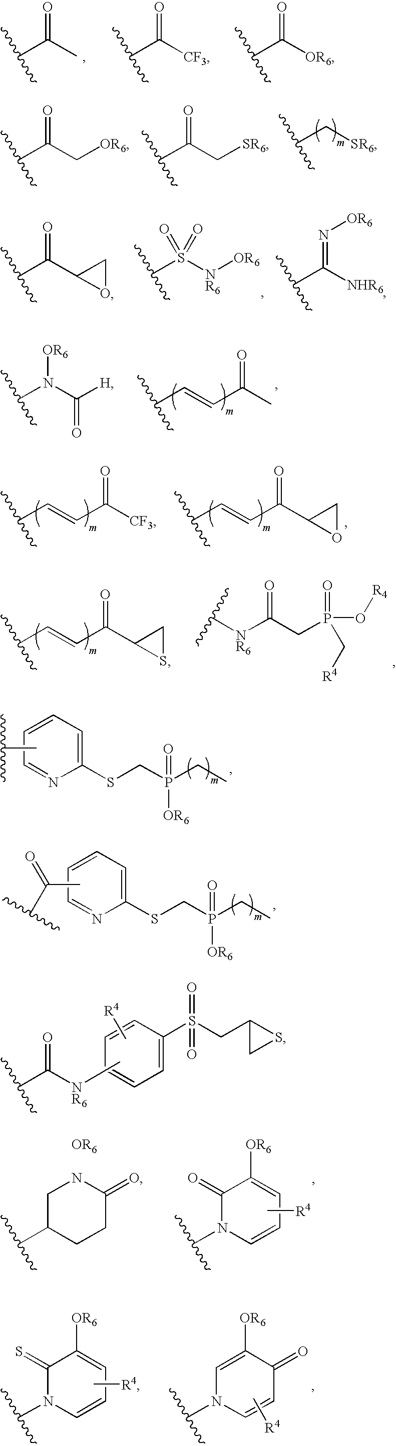 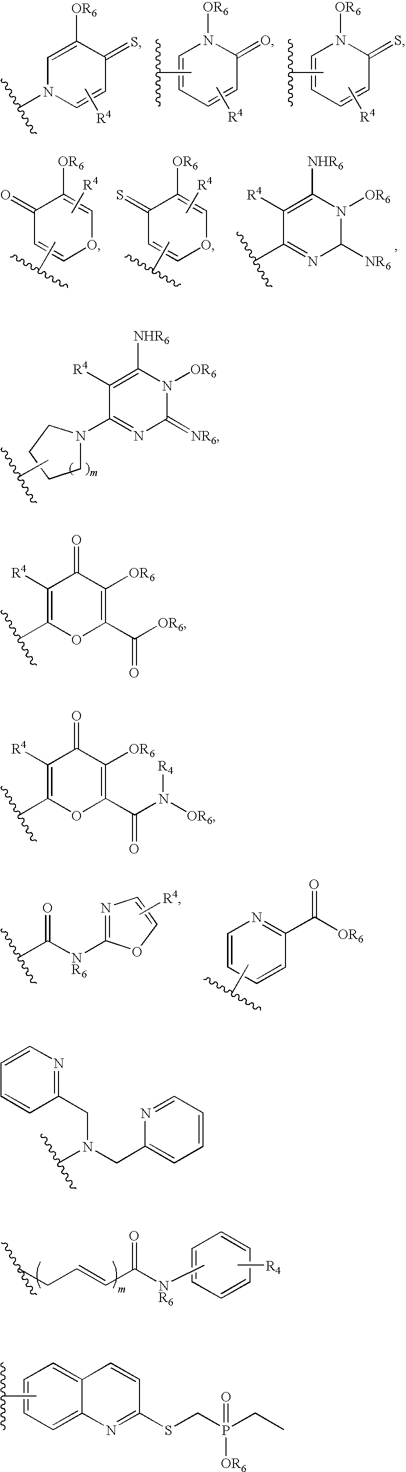 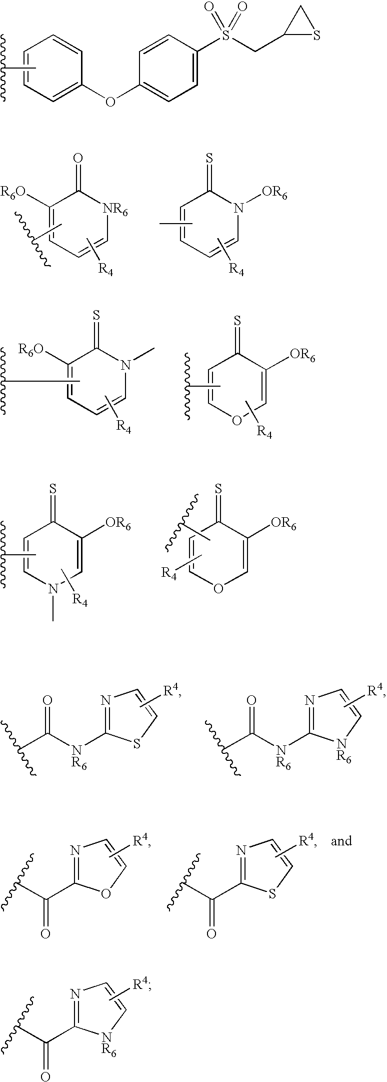
wherein
R⁴ is hydrogen, halogen, —CN, a water solubilizing group, -L-OH, -L-NH₂, or a substituted or unsubstituted group selected from -L-alkyl, L-alkenyl, L-alkynyl, -L-cycloalkyl, L-cycloalkenyl, -L-heterocycloalkyl, -L-haloalkyl, -L-alkoxy, -L-alkylamine, -L-dialkylamine, -L-aryl, and -L-heteroaryl, wherein L is a bond, —C(O)—, —S(O), and —S(O)₂, wherein the water solubilizing group is: 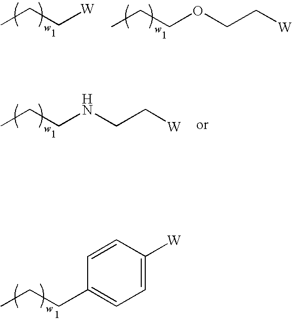
where W is selected from: 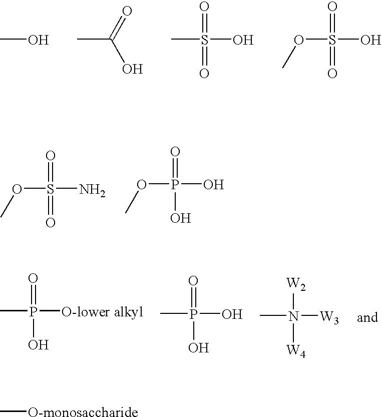
where W₁ is 0, 1, 2, or 3; W₂ and W₃ are each independently hydrogen or methyl or, when taken together with the nitrogen to which they are attached, W₂ and W₃ form a five or six membered ring that optionally contains an oxygen atom or a second nitrogen atom; and W₄ is an electron pair or an oxygen atom; and
R⁶ is hydrogen, lower alkyl, lower acyl, lower alkoxycarbonyl, carbamoyl, —PO₃H₂, or —SO₃H; and
m=0 or 1 or 2,
provided that the compound is not:

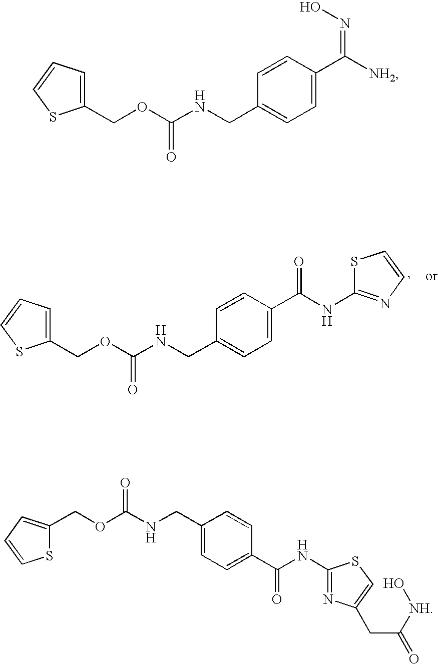

75. The method of claim 74, for inhibiting tumor size increase or reducing the size of a tumor wherein said tumor occurs in the brain, breast, lung, ovaries, pancreas, prostate, kidney, colon or rectum.

76. The method of claim 74, wherein said compound of Formula I is administered in combination with an additional cancer therapy.

77. The method of claim 76, wherein the additional cancer therapy is selected from surgery, radiation therapy, and administration of at least one chemotherapeutic agent.

78. The method of claim 76, wherein said additional cancer therapy is administration of at least one chemotherapeutic agent.

79. The method of claim 74, wherein the administration of said compound of Formula I occurs after surgery.

80. The compound of claim 1, wherein the compound is an HDAC modulator.

81. The compound of claim 1, wherein the compound is a selective HDAC modulator.
